(19) United States
(12) Patent Application Publication
(10) Pub. No.: US 2005/0143391 A1
(43) Pub. Date: Jun. 30, 2005

(54) Heterocyclic amides with alpha-4 integrin antagonist activity

(75) Inventors: Elena Carceller Gonzalez, San Cugat del Valles, Barcelona (ES); Jorge Salas Solana, Granollers (ES); Cristina Alvarez Farrerons, Barcelona (ES); Juan Andres Andreu Azanza, Barcelona (ES)

Correspondence Address: ROTHWELL, FIGG, ERNST & MANBECK, P.C., 1425 K STREET, N.W., SUITE 800, WASHINGTON, DC, 20005, US

(73) Assignee: J. Uriach Y Compañia S.A., Palau-Solita i Plegamans (ES)

(21) Appl. No.: 10/510,626

(22) PCT Filed: Apr. 8, 2003
(86) PCT No.: PCT/EP03/03635

(30) Foreign Application Priority Data
Apr. 8, 2002 (ES) 2002 00805

(51) Int. Cl. A61K 31/496; A61K 31/506; A61K 31/454; C07D 413/04; C07D 43/02

(52) U.S. Cl. 514/254.01; 514/326; 544/367; 544/372; 546/208; 546/209; 544/333; 514/256; 514/254.04

(57) ABSTRACT

The present invention relates to new compounds of Formula (I) and the salts, solvates and prodrugs thereof, wherein the meanings for the various substituents are as disclosed in the description. These compounds are useful as integrin α₄ antagonists.

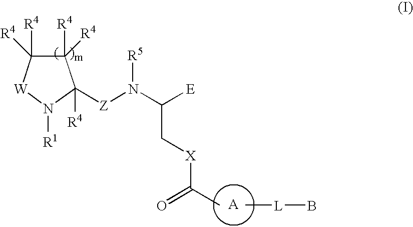

### FIELD OF THE INVENTION

The present invention relates to a new series of compounds with α₄ integrin antagonist activity, as well as to a process for their preparation, to the pharmaceutical compositions that contain these compounds and to their use in medicine.

### BACKGROUND OF THE INVENTION

Cell adhesion is a process by which cells bind either to other cells or to extracellular matrix through an extracellular ligand. The cell-extracellular ligand binding triggers in its turn several processes related to signal transduction, cell cycle and apoptosis, which are involved in processes of great physiological relevance. Thus, cell adhesion takes part in processes such as embryological development, angiogenesis or platelet aggregation and is also involved in pathological processes, such as inflammation and metastasis.

Cell-cell and cell-extracellular matrix interactions are mediated by different molecule families such as the integrins. Integrins are heterodimeric receptors that are anchored in the cell membrane and that consist of an a subunit and a β subunit. Up to now, 16 α subunits (α₁₋₁₀, α-L, α-M, α-X, α-IIb, α-V and α-E) and 8 β subunits (β₁₋₈), which give rise to 24 combinations of integrins, have been identified. The α₄ subunit combines only with the β₁ and β₇ subunits, giving rise to the α₄β₁ (also called Very Late Antigen 4, VLA-4, or CD49d/CD29) and α₄β₇ (or Lymphocyte Peyer's patch cell Adhesion Molecule, LPAM-1) integrins.

The integrin α₄β₁ is widely expressed on mononuclear leukocytes, mast cells, macrophages, basophils and eosinophils, and on neutrophils as well but with a low level of functionality. The integrin α₄β₇ is only expressed on some types of lymphocytes. The integrin α₄β₁ binds to VCAM-1 (Vascular Cell Adhesion Molecule-1), which is a membrane protein of endothelial cells induced by pro-inflammatory cytokines, through the sequence Gln-Ile-Asp-Ser. It also binds to the sequence Leu-Asp-Val of the CS-1 domain of fibronectin, an extracellular matrix molecule. The integrin α₄β₇ also binds to the constitutive immunoglobulin MadCAM (Mucosal addressing Cell Adhesion Molecule) and is responsible for the attraction of lymphocytes to the intestinal and mesenteric lymph nodes.

Integrins α₄, by means of their binding to the above-mentioned ligands, are involved in numerous cell adhesion pathological processes such as asthma, multiple sclerosis, rheumatoid arthritis and inflammatory bowel disease, among others. Compounds which antagonize the action of these integrins are therefore expected to be useful for the treatment or prevention of said diseases. The present invention describes new compounds with α₄ integrin antagonist activity.

### DESCRIPTION OF THE INVENTION

An aspect of the present invention relates to the new compounds of general formula I: 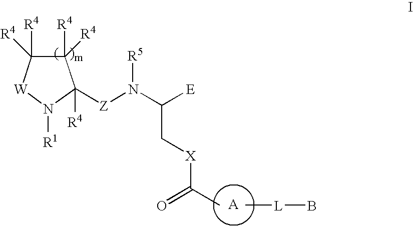
wherein:
R¹ represents —SO₂R², —COR² or —CH₂R³;
R² represents C₁₋₈ alkyl, C₂₋₈ alkenyl or C₂₋₈ alkynyl, which can be optionally substituted with one or more groups R^{a}, or R² represents Cy, CyC₁₋₄ alkyl, CyC₂₋₄ alkenyl or CyC₂₋₄ alkynyl, where the groups Cy can be optionally substituted with one or more groups R^{b};
R³ represents hydrogen, C₁₋₈ alkyl, C₂₋₈ alkenyl or C₂₋₈ alkynyl, where the groups C₁₋₈ alkyl, C₂₋₈ alkenyl and C₂₋₈ alkynyl can be optionally substituted with one or more groups R^{c}, or R³ represents Cy or CyC₁₋₄ alkyl, where the groups Cy can be optionally substituted with one or more groups selected from R^{c} and R^{d};
each R⁴ independently represents hydrogen, C₁₋₈ alkyl, Cy or CyC₁₋₄ alkyl, where the C₁₋₈ alkyl group can be optionally substituted with one or more groups R^{c} and where the groups Cy can be optionally substituted with one or more groups selected from R^{c} and R^{d};
W represents —CR⁴R⁴— when R¹ is —SO₂R² or —COR², or W represents —CO— when R¹ is —CH₂R³;
Z represents —CO— or —CS—;
E represents —COOR⁶, —CONR⁷R⁸ or 5-tetrazolyl;
X represents —CH₂—, —NR⁵— or —O—;
each R⁵ independently represents hydrogen or C₁₋₄ alkyl;
R⁶ represents hydrogen, C₁₋₈ alkyl, C₃₋₇ cycloalkyl or aryl, where the C₁₋₈ alkyl group can be optionally substituted with a group selected from C₃₋₇ cycloalkyl, aryl, —OR^{g}, —OCOR^{d}, —OCOOR^{d}, —COOR^{g} and —NHCOR^{g} and the aryl groups can be optionally substituted with one or more groups R^{b};
R⁷ represents hydrogen, C₁₋₈ alkyl, C₃₋₇ cycloalkyl, aryl or —SO₂R^{d}, where the C₁₋₈ alkyl group can be optionally substituted with a group selected from C₃₋₇ cycloalkyl, aryl, —SO₂R^{d}, —COOR^{g} and —COR^{d};
R⁸ represents hydrogen or C₁₋₈ alkyl;
or R⁷ and R⁸ together with the nitrogen atom to which they are bound can form a cycle Het¹;
A represents C₃₋₇ cycloalkyl or Het¹, which can be optionally substituted with one or more groups selected from oxo, C₁₋₈ alkyl and C₁₋₈ haloalkyl;
L represents —(CR⁹R^{g})ₙ—;
each R⁹ independently represents hydrogen or C₁₋₄ alkyl;
B represents:
i) C₃₋₇ cycloalkyl, Het¹ or Het², which can be optionally substituted with one or more groups selected from oxo, R^{b} and Cy optionally substituted with one or more groups R^{b}; or
ii) a group selected from —COR^{e}, —NR^{f}R^{f}, —OR^{f}, —SR^{f}, —S(O)ₚR^{e}, —CONR^{f}R^{f}, —NR^{f}COR^{f}, —NR^{f}CONR^{f}R^{f}, —NR^{f}CSNR^{f}R^{f}, —NR^{f}COOR^{e}, —OCOR^{e}, —OCONR^{f}R^{f}, —NR^{f}SO₂R^{e} and —SO₂NR^{f}R^{f};
m represents 0 or 1;
n represents 1, 2, 3 or 4;
p represents 1 or 2;
each R^{a} independently represents halogen, —COR^{d}, —OR^{g}, —NR^{g}R^{g}, —COOR^{g}, —OCOR^{d}, —CONR^{g}R^{g}, —NR^{g}COR^{g}, —OCONR^{g}R^{g} or —NR^{g}COOR^{d};
each R^{b} independently represents a group R^{a}, —NO₂, —SR^{g}, —S(O)ₚR^{d} or C₁₋₈ alkyl optionally substituted with one or more groups R^{c};
each R^{c} independently represents halogen, —OR^{h} or —NR^{h}R^{h};
each R^{d} independently represents C₁₋₈ alkyl, C₃₋₇ cycloalkyl or aryl, which can be optionally substituted with one or more groups R^{c};
each R^{e} independently represents C₁₋₈ alkyl, C₂₋₈ alkenyl or C₂₋₈ alkynyl, which can be optionally substituted with one or more groups R^{a}, or R^{e} represents Cy or CyC₁₋₄ alkyl, where the groups Cy can be optionally substituted with one or more groups selected from oxo, Cy* and R^{b}, and where the groups Cy* can be optionally substituted with one or more groups selected from oxo and R^{b};
each R^{f} independently represents hydrogen or any of the meanings described for R^{e};
or two groups R^{f} placed on the same nitrogen atom can be attached to each other to form together with said nitrogen atom a cycle Het¹ which can be optionally substituted with one or more groups selected from oxo, Cy and R^{b}, where the groups Cy can be optionally substituted with one or more groups selected from oxo and R^{b};
each R^{g} independently represents hydrogen or any of the meanings described for R^{d};
or two groups R^{g} placed on the same nitrogen atom can be attached to each other to form together with said nitrogen atom a cycle Het¹ which can be optionally substituted with one or more groups selected from oxo, Cy and R^{b}, where the groups Cy can be optionally substituted with one or more groups selected from oxo and R^{b};
each R^{h} independently represents hydrogen, C₁₋₈ alkyl, C₃₋₇ cycloalkyl or aryl, where the groups C₁₋₈ alkyl, C₃₋₇ cycloalkyl and aryl can be optionally substituted with one or more halogen atoms;
Cy and Cy* independently represent aryl, C₃₋₇ cycloalkyl, Het¹ or Het²;
aryl in the above definitions represents phenyl or naphthyl;
Het¹ in the above definitions represents a saturated or insaturated non-aromatic 5- to 7-membered monocyclic ring containing from one to four heteroatoms selected from N, O and S, which can be optionally fused to a phenyl, naphthyl or Het² ring, and which is chemically stable and obtainable through chemical synthesis; and
Het² in the above definitions represents an aromatic 5- to 7-membered monocyclic or 9- to 11-membered bicyclic ring, which contains from one to four heteroatoms selected from N, O and S, and which is chemically stable and obtainable through chemical synthesis.

The present invention also relates to the addition salts of the compounds of the invention, as well as to their solvates and prodrugs.

Some compounds of formula I can have chiral centres, which can give rise to various stereoisomers. The present invention relates to each one of the individual stereoisomers as well as to their mixtures. Moreover, some of the compounds of the present invention can show cis/trans isomery. The present invention relates to each one of the geometric isomers as well as to their mixtures.

Another aspect of the present invention relates to the pharmaceutical compositions which comprise an effective amount of a compound of formula I or a pharmaceutically acceptable salt, solvate or prodrug thereof and one or more pharmaceutically acceptable excipients.

Another aspect of the present invention relates to the use of a compound of formula I or a pharmaceutically acceptable salt, solvate or prodrug thereof for the manufacture of a medicament for the treatment or prevention of diseases mediated by integrins α₄. In a preferred embodiment, said α₄ integrin-mediated disease is selected from the group consisting of: inflammatory diseases, immune diseases, autoimmune diseases, degenerative disorders, tumor metastasis and ischemia-reperfusion disorders.

Another aspect of the present invention relates to the use of a compound of formula I or a pharmaceutically acceptable salt, solvate or prodrug thereof for the manufacture of a medicament for the treatment or prevention of inflammatory, immune and/or autoimmune diseases. In a preferred embodiment, said inflammatory, immune and/or autoimmune disease is selected from: diseases with an allergic component, such as for example asthma, allergic rhinitis, allergic dermatitis and allergic conjunctivitis; inflammatory diseases with an autoimmune component, such as for example rheumatoid arthritis, psoriatic arthritis, multiple sclerosis, psoriasis and diabetes; inflammatory bowel disease, including Crohn's disease and ulcerative colitis; inflammatory processes having an alloimmune origin caused by transplants or rejections; inflammatory processes that develop as a consequence of blood vessel revascularization treatments, such as percutaneous transluminal coronary angioplasty; encephalomyelitis; hepatitis; bronchitis; vasculitis; and atherosclerosis.

Another aspect of the present invention relates to the use of a compound of formula I or a pharmaceutically acceptable salt, solvate or prodrug thereof for the manufacture of a medicament for the treatment or prevention of degenerative disorders, such as Alzheimer's disease and arthrosis.

Another aspect of the present invention relates to the use of a compound of formula I or a pharmaceutically acceptable salt, solvate or prodrug thereof for the manufacture of a medicament for the treatment or prevention of tumor metastasis.

Another aspect of the present invention relates to the use of a compound of formula I or a pharmaceutically acceptable salt, solvate or prodrug thereof for the manufacture of a medicament for the treatment or prevention of ischemia-reperfusion disorders, including acute coronary diseases and stroke.

Another aspect of the present invention relates to a compound of formula I or a pharmaceutically acceptable salt, solvate or prodrug thereof for the treatment or prevention of diseases mediated by integrins α₄. In a preferred embodiment, said α₄ integrin-mediated disease is selected from the group consisting of: inflammatory diseases, immune diseases, autoimmune diseases, degenerative disorders, tumor metastasis and ischemia-reperfusion disorders.

Another aspect of the present invention relates to a compound of formula I or a pharmaceutically acceptable salt, solvate or prodrug thereof for the treatment or prevention of inflammatory, immune and/or autoimmune diseases. In a preferred embodiment, said inflammatory, immune and/or autoimmune disease is selected from: diseases with an allergic component, such as for example asthma, allergic rhinitis, allergic dermatitis and allergic conjunctivitis; inflammatory diseases with an autoimmune component, such as for example rheumatoid arthritis, psoriatic arthritis, multiple sclerosis, psoriasis and diabetes; inflammatory bowel disease, including Crohn's disease and ulcerative colitis; inflammatory processes having an alloimmune origin caused by transplants or rejections; inflammatory processes that develop as a consequence of blood vessel revascularization treatments, such as percutaneous transluminal coronary angioplasty; encephalomyelitis; hepatitis; bronchitis; vasculitis; and atherosclerosis.

Another aspect of the present invention relates to a compound of formula I or a pharmaceutically acceptable salt, solvate or prodrug thereof for the treatment or prevention of degenerative disorders, such as Alzheimer's disease and arthrosis.

Another aspect of the present invention relates to a compound of formula I or a pharmaceutically acceptable salt, solvate or prodrug thereof for the treatment or prevention of tumor metastasis.

Another aspect of the present invention relates to a compound of formula I or a pharmaceutically acceptable salt, solvate or prodrug thereof for the treatment or prevention of ischemia-reperfusion disorders, including acute coronary diseases and stroke.

Another aspect of the present invention relates to the use of a compound of formula I or a pharmaceutically acceptable salt, solvate or prodrug thereof for the treatment or prevention of diseases mediated by integrins α₄. In a preferred embodiment, said α₄ integrin-mediated disease is selected from the group consisting of: inflammatory diseases, immune diseases, autoimmune diseases, degenerative disorders, tumor metastasis and ischemia-reperfusion disorders.

Another aspect of the present invention relates to the use of a compound of formula I or a pharmaceutically acceptable salt, solvate or prodrug thereof for the treatment or prevention of inflammatory, immune and/or autoimmune diseases. In a preferred embodiment, said inflammatory, immune and/or autoimmune disease is selected from: diseases with an allergic component, such as for example asthma, allergic rhinitis, allergic dermatitis and allergic conjunctivitis; inflammatory diseases with an autoimmune component, such as for example rheumatoid arthritis, psoriatic arthritis, multiple sclerosis, psoriasis and diabetes; inflammatory bowel disease, including Crohn's disease and ulcerative colitis; inflammatory processes having an alloimmune origin caused by transplants or rejections; inflammatory processes that develop as a consequence of blood vessel revascularization treatments, such as percutaneous transluminal coronary angioplasty; encephalomyelitis; hepatitis; bronchitis; vasculitis; and atherosclerosis.

Another aspect of the present invention relates to the use of a compound of formula I or a pharmaceutically acceptable salt, solvate or prodrug thereof for the treatment or prevention of degenerative disorders, such as Alzheimer's disease and arthrosis.

Another aspect of the present invention relates to the use of a compound of formula I or a pharmaceutically acceptable salt, solvate or prodrug thereof for the treatment or prevention of tumor metastasis.

Another aspect of the present invention relates to the use of a compound of formula I or a pharmaceutically acceptable salt, solvate or prodrug thereof for the treatment or prevention of ischemia-reperfusion disorders, including acute coronary diseases and stroke.

Another aspect of the present invention relates to a method of treating or preventing diseases mediated by integrins α₄ in a mammal in need thereof, especially a human being, which comprises administering to said mammal a therapeutically effective amount of a compound of formula I or a pharmaceutically acceptable salt, solvate or prodrug thereof. In a preferred embodiment, said α₄ integrin-mediated disease is selected from the group consisting of: inflammatory diseases, immune diseases, autoimmune diseases, degenerative disorders, tumor metastasis and ischemia-reperfusion disorders.

Another aspect of the present invention relates to a method of treating or preventing inflammatory, immune and/or autoimmune diseases in a mammal in need thereof, especially a human being, which comprises administering to said mammal a therapeutically effective amount of a compound of formula I or a pharmaceutically acceptable salt, solvate or prodrug thereof. In a preferred embodiment, said inflammatory, immune and/or autoimmune disease is selected from: diseases with an allergic component, such as for example asthma, allergic rhinitis, allergic dermatitis and allergic conjunctivitis; inflammatory diseases with an autoimmune component, such as for example rheumatoid arthritis, psoriatic arthritis, multiple sclerosis, psoriasis and diabetes; inflammatory bowel disease, including Crohn's disease and ulcerative colitis; inflammatory processes having an alloimmune origin caused by transplants or rejections; inflammatory processes that develop as a consequence of blood vessel revascularization treatments, such as percutaneous transluminal coronary angioplasty; encephalomyelitis; hepatitis; bronchitis; vasculitis; and atherosclerosis.

Another aspect of the present invention relates to a method of treating or preventing degenerative disorders, such as Alzheimer's disease and arthrosis, in a mammal in need thereof, especially a human being, which comprises administering to said mammal a therapeutically effective amount of a compound of formula I or a pharmaceutically acceptable salt, solvate or prodrug thereof.

Another aspect of the present invention relates to a method of treating or preventing tumor metastasis in a mammal in need thereof, especially a human being, which comprises administering to said mammal a therapeutically effective amount of a compound of formula I or a pharmaceutically acceptable salt, solvate or prodrug thereof.

Another aspect of the present invention relates to a method of treating or preventing ischemia-reperfusion disorders, including acute coronary diseases and stroke, in a mammal in need thereof, especially a human being, which comprises administering to said mammal a therapeutically effective amount of a compound of formula I or a pharmaceutically acceptable salt, solvate or prodrug thereof.

Another aspect of the present invention relates to a process for preparing the compounds of formula 1, which comprises:
(a) when in a compound of formula I X represents —NR⁵—, reacting an amine of formula II with an acid of formula III 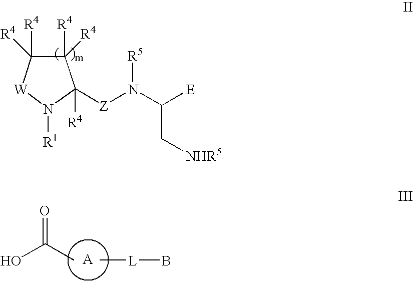
   wherein R¹, R⁴, R⁵, W, Z, E, A, L, B and m have the meaning described above; or
(b) when in a compound of formula I X represents —O—, reacting an alcohol of formula IV with an acid of formula III 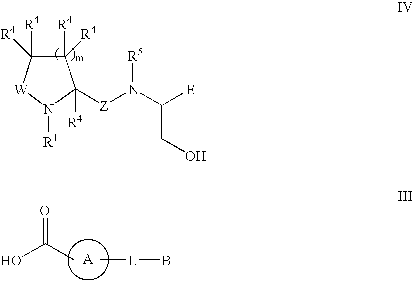
   wherein R¹, R⁴, R⁵, W, Z, E, A, L, B and m have the meaning described above; or
(c) when in a compound of formula I X represents —CH₂— and cycle A is bound to the carbonyl group through a nitrogen atom, reacting an acid of formula V with an amine of formula VI 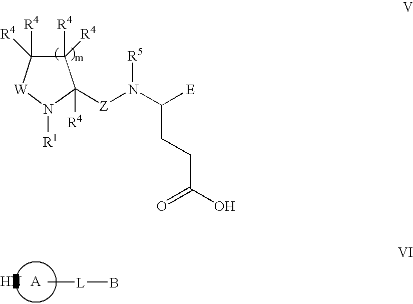
   wherein R¹, R⁴, R⁵, W, Z, E, A, L, B and m have the meaning described above; or
(d) when in a compound of formula I X represents —NR⁵— and cycle A is bound to the carbonyl group through a nitrogen atom, reacting an amine of formula II previously activated with an activating agent suitable for the preparation of ureas with an amine of formula VI 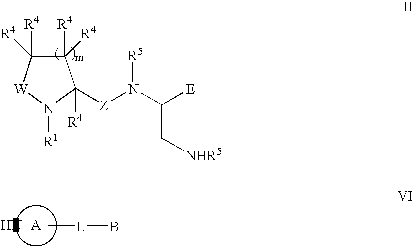
   wherein R¹, R⁴, R⁵, W, Z, E, A, L, B and m have the meaning described above, or reacting an amine of formula VI previously activated with an activating agent suitable for the preparation of ureas with an amine of formula II, or alternatively reacting a compound of formula V′ previously activated with an azide suitable for a Curtius rearrangement with an amine of formula VI 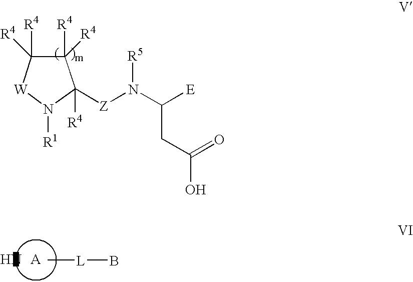
   wherein R¹, R⁴, R⁵, W, Z, E, A, L, B and m have the meaning described above; or
(e) when in a compound of formula I X represents —O— and cycle A is bound to the carbonyl group through a nitrogen atom, reacting an alcohol of formula IV previously activated with an activating agent suitable for the preparation of carbamates with an amine of formula VI 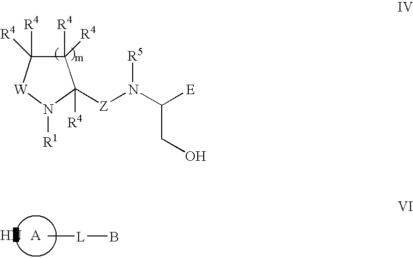
   wherein R¹, R⁴, R⁵, W, Z, E, A, L, B and m have the meaning described above; or
(f) when in a compound of formula I Z represents —CO—, reacting an acid of formula VII with an amine of formula XVII 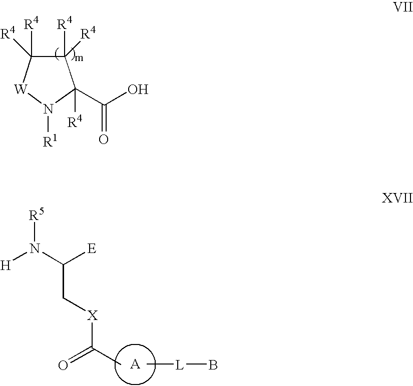
   wherein R¹, R⁴, R⁵, W, E, X, A, L, B and m have the meaning described above; or
(g) when in a compound of formula I W represents —CR⁴R⁴— and R¹ represents —SO₂R², reacting a compound of formula XVIII with a sulfonyl chloride of formula IX 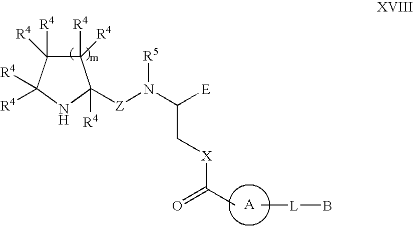
   wherein R², R⁴, R⁵, Z, E, X, A, L, B and m have the meaning described above; or
(h) when in a compound of formula I W represents —CR⁴R⁴— and R¹ represents —COR², reacting a compound of formula XVIII with an acid of formula X 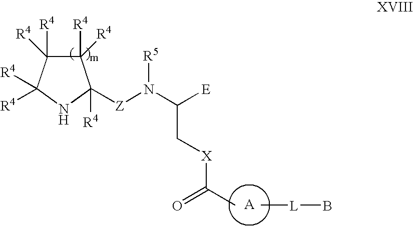
   wherein R², R⁴, R⁵, Z, E, X, A, L, B and m have the meaning described above; or
(i) when in a compound of formula I W represents —CO— and R¹ represents —CH₂R³, reacting a compound of formula XIX with a compound of formula XI 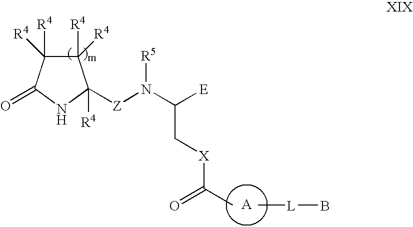
   wherein R², R⁴, R⁵, Z, E, X, A, L, B and m have the meaning described above and D represents a good leaving group; or
(j) when in a compound of formula I cycle A is bound to the -L-B moiety through a ring nitrogen atom, alkylating the secondary amine of a compound of formula XX with a compound of formula XIV 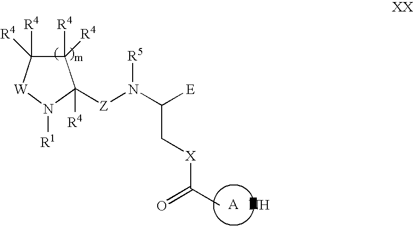
   wherein R¹, R⁴, R⁵, W, Z, E, X, A, L, B and m have the meaning described above and D represents a good leaving group; or
(k) transforming, in one or a plurality of steps, a compound of formula I into another compound of formula I; and
(l) if desired, after the above steps, reacting a compound of formula I with an acid or a base to give the corresponding addition salt.

In the above definitions, the term C₁₋₄ or C₁₋₈ alkyl, as a group or part of a group, means a linear or branched alkyl group that contains from 1 to 4 or from 1 to 8 carbon atoms, respectively. Examples include among others the groups methyl, ethyl, propyl, isopropyl, butyl, isobutyl, sec-butyl, tert-butyl, pentyl, isopentyl, neopentyl, hexyl, heptyl and octyl.

A C₂₋₄ or C₂₋₈ alkenyl group, as a group or part of a group, means a linear or branched alkyl chain that contains from 2 to 4 or from 2 to 8 carbon atoms, respectively, and that in addition contains one or more double bonds. Examples include among others the groups ethenyl, 1-propenyl, 2-propenyl, isopropenyl, 1-butenyl, 2-butenyl, 3-butenyl, 1,3-butadienyl, 1-pentenyl, 2-pentenyl, 3-pentenyl, 4-pentenyl, 1-hexenyl, 2-hexenyl, 3-hexenyl, 4-hexenyl, 5-hexenyl, 6-heptenyl or 7-octenyl.

A C₂₋₄ or C₂₋₈ alkynyl group, as a group or part of a group, means a linear or branched alkyl chain which contains from 2 to 4 or from 2 to 8 carbon atoms, respectively, and that in addition contains one or more triple bonds. Examples include the groups ethynyl, 1-propynyl, 2-propynyl, 1-butynyl, 2-butynyl, 3-butynyl, 2-pentynyl, 3-pentynyl, 4-pentynyl, 2-hexynyl, 3-hexynyl, 4-hexynyl, 5-hexynyl, 6-heptynyl or 7-octynyl.

A halogen radical or its abbreviation halo means fluoro, chloro, bromo or iodo.

An oxo group means a carbonyl group (—CO—).

A C₁₋₈ haloalkyl group means a group resulting from the substitution of one or more hydrogen atoms of a C₁₋₈ alkyl group with one or more halogen atoms (that is, fluoro, chloro, bromo or iodo), which can be the same or different. Examples include trifluoromethyl, fluoromethyl, 1-chloroethyl, 2-chloroethyl, 1-fluoroethyl, 2-fluoroethyl, 2-bromoethyl, 2-iodoethyl, 2,2,2-trifluoroethyl, pentafluoroethyl, 3-fluoropropyl, 3-chloropropyl, 2,2,3,3-tetrafluoropropyl, 2,2,3,3,3-pentafluoropropyl, heptafluoropropyl, 4-fluorobutyl, nonafluorobutyl, 5-fluoropentyl, 6-fluorohexyl, 7-fluoroheptyl and 8-fluorooctyl.

The term Cy or Cy*, as a group or part of a group, means aryl, C₃₋₇ cycloalkyl, Het¹ or Het², which can be optionally substituted as mentioned above in the definition of general formula I and where said substituents, if present, can be on any available position of the Cy or Cy* groups.

The term aryl means phenyl or naphthyl.

A group C₃₋₇ cycloalkyl represents cyclopropyl, cyclobutyl, cyclopentyl, cyclohexyl or cycloheptyl.

The term Het¹ means a saturated or insaturated non-aromatic 5- to 7-membered monocyclic ring containing from one to four heteroatoms selected from N, O and S, and which is chemically stable and obtainable through chemical synthesis. Said cycle can be bound to the rest of the molecule through a carbon or nitrogen atom and can be optionally fused to a phenyl, naphthyl or Het² ring. Examples of heterocycles Het¹ include among others, piperidine, piperazine, pyrrolidine, pyrazolidine, imidazolidine, morpholine, dioxane, thiazolidine, isothiazolidine, oxazolidine, isoxazolidine and homopiperazine.

The term Het² means an aromatic 5- to 7-membered monocyclic or 9- to 11-membered bicyclic ring, which contains from one to four heteroatoms selected from N, O and S, and which is chemically stable and obtainable through chemical synthesis. Examples of monocyclic aromatic heterocycles include thiophene, furan, pyrrole, thiazole, isothiazole, oxazole, isoxazole, imidazole, pyrazole, 1,2,3-triazole, 1,2,4-triazole, 1,3,4-oxadiazole, 1,3,4-thiadiazole, 1,2,4-oxadiazole, 1,2,4-thiadiazole, pyridine, pyrazine, pyrimidine and pyridazine. Examples of bicyclic heteroaryl groups include among others benzimidazole, benzofuran, indole, isoindole, benzothiophene, benzothiazole, quinoline, isoquinoline, phthalazine, quinazoline, quinoxaline, cinnoline, naphthyridine, indazole, imidazopyridine, imidazopyrimidine, imidazopyrazine, imidazopyridazine, pyrazolopyrazine, pyrazolopyridine and pyrazolopyrimidine. The examples of bicyclic heteroaryls mentioned above whose name can have more than one possible interpretation are to be understood as including all the possible fusion combinations between the monocycles that constitute them.

A CyC₁₋₄ alkyl group means a group resulting from the substitution of a hydrogen atom of a C₁₋₄ alkyl group with a Cy defined above, that is with an aryl, C₃₋₇ cycloalkyl, Het¹ or Het² group. Examples include among others benzyl, 1-phenylethyl, 2-phenylethyl, 3-phenylpropyl, 4-phenylbutyl, cyclohexylmethyl, 1-cyclohexylethyl, 2-cyclohexylethyl, 3-cyclohexylpropyl, 4-cyclohexylbutyl, 2-pyridylmethyl, 3-pyridylmethyl, 4-pyridylmethyl, 1-(4-pyridyl)ethyl, 2-(4-pyridyl)ethyl, 3-(4-pyridyl)propyl, 4-(4-pyridyl)butyl, 1-piperidylmethyl, 2-piperidylmethyl, 3-piperidylmethyl, 4-piperidylmethyl, 2-(4-piperidyl)ethyl, 3-(4-piperidyl)propyl, 4-(4-piperidyl)butyl, 1-piperazinylmethyl, 2-piperazinylmethyl, 1-(1-piperazinyl)ethyl, 2-(1-piperazinyl)ethyl, 3-(1-piperazinyl)propyl and 4-(1-piperazinyl)butyl.

A CyC₂₋₄ alkenyl group means a group resulting from the substitution of a hydrogen atom of a C₂₋₄ alkenyl group with a Cy, that is with an aryl, C₃₋₇ cycloalkyl, Het¹ or Het² group. Examples include among others 1-phenylethenyl, 2-phenylethenyl, 3-phenyl-1-propenyl, 3-phenyl-2-propenyl, 4-phenyl-1-butenyl, 4-phenyl-1,3-butandienyl, 2-cyclohexylethenyl, 3-cyclohexyl-2-propenyl, 4-cyclohexyl-2-butenyl, 2-(2-pyridyl)ethenyl, 2-(3-pyridyl)ethenyl, 2-(4-pyridyl)ethenyl, 3-(4-pyridyl)-2-propenyl, 4-(4-pyridyl)-3-butenyl, 2-(1-piperazinyl)ethenyl, 3-(1-piperazinyl)-2-propenyl, 4-(1-piperazinyl)-2-butenyl, 2-(1-piperidyl)ethenyl, 2-(4-piperidyl)ethenyl, 3-(1-piperidyl)-2-propenyl and 4-(1-piperidyl)-3-butenyl.

A CyC₂₋₄ alkynyl group means a group resulting from the substitution of a hydrogen atom of a C₂₋₄ alkynyl group with a Cy, that is with an aryl, C₃₋₇ cycloalkyl, Het¹ or Het² group. Examples include among others phenylethynyl, 3-phenyl-2-propynyl, 4-phenyl-3-butynyl, cyclohexylethynyl, 3-cyclohexyl-2-propynyl, 4-cyclohexyl-2-butynyl, 2-pyridylethynyl, 3-pyridylethynyl, 4-pyridylethynyl, 3-(2-pyridyl)-2-propynyl, 3-(3-pyridyl)₂-propynyl, 3-(4-pyridyl)₂-propynyl, 4-(4-pyridyl)-3-butynyl, 1-piperazinylethynyl, 3-(1-piperazinyl)-2-propynyl, 4-(1-piperazinyl)-3-butynyl, 1-piperidylethynyl, 4-piperidylethynyl, 3-(4-piperidyl)-2-propynyl and 4-(4-piperidyl-3-butynyl.

The sentence “substituted with one or more” means the possibility of a group being substituted with one or more, preferably with 1, 2, 3 or 4 substituents, as long as said group has 1, 2, 3 or 4 positions capable of being substituted.

Although the present invention includes all the compounds mentioned above, those compounds of formula I wherein R¹ represents —SO₂R² are preferred.

Those compounds of formula I wherein R² represents aryl optionally substituted with one or more groups R^{b} are also preferred.

Those compounds of formula I wherein all the groups R⁴ represent hydrogen are also preferred.

Those compounds of formula I wherein R⁵ represents hydrogen are also preferred.

Those compounds of formula I wherein W represents —CR⁴R⁴—, preferably —CH₂—, are also preferred.

Those compounds of formula I wherein Z represents —CO— are also preferred.

Those compounds of formula I wherein E represents —COOR⁶, preferably —COOH, are also preferred.

Those compounds of formula I wherein m represents 1 are also preferred.

Another preferred group of compounds of formula I are those compounds wherein X represents —NH—.

Another preferred group of compounds of formula I are those compounds wherein X represents —CH₂—.

Another preferred group of compounds of formula I are those compounds wherein X represents —O—.

Those compounds of formula I wherein A represents piperidine or piperazine are also preferred.

Those compounds of formula I wherein L represents —(CH₂)ₙ—, preferably methylene or ethylene, are also preferred.

Those compounds of formula I wherein B represents Het¹ or Het² optionally substituted with one or more groups selected from oxo, R^{b} and Cy optionally substituted with one or more groups R^{b} are also preferred. More preferably, B represents imidazopyridine optionally substituted with one or more groups selected from oxo, R^{b} and Cy optionally substituted with one or more groups R^{b}.

Those compounds of formula I wherein B represents —NR^{f}R^{f}, —OR^{f}, —NR^{f}COR^{f}, —NR^{f}CONR^{f}R^{f}, —NR^{f}CSNR^{f}R^{f}, —NR^{f}COOR^{e} or —OCONR^{f}R^{f} are also preferred. More preferably, B represents —OCONR^{f}R^{f}, and still more preferably B represents —OCONR^{f}R^{f} wherein both R^{f} groups are attached to each other to form together with the nitrogen atom a cycle Het¹ which can be optionally substituted with one or more groups selected from oxo, Cy and R^{b}, wherein the groups Cy can be optionally substituted with one or more groups selected from oxo and R^{b}.

Accordingly, a preferred embodiment of the present invention are the compounds of formula I wherein:

R¹ represents —SO₂R²;
W represents —CR⁴R⁴—;
Z represents —CO—;
E represents —COOR⁶;
m represents 1;
and R², R⁴, R⁵, R⁶, X, A, L and B have the meaning described above.

Another preferred embodiment of the present invention are the compounds of formula I wherein:

R¹ represents —SO₂R²;
R² represents aryl optionally substituted with one or more groups R^{b};
each R⁴ represents hydrogen;
R⁵ represents hydrogen;
W represents —CH₂—;
Z represents —CO—;
E represents —COOR⁶;
m represents 1;
and R⁶, R^{b}, X, A, L and B have the meaning above described.

Another preferred embodiment of the present invention are the compounds of formula I wherein:

R¹ represents —SO₂R²;
R² represents aryl optionally substituted with one or more groups R^{b};
each R⁴ represents hydrogen;
R⁵ represents hydrogen;
W represents —CH₂—;
Z represents —CO—;
E represents —COOR⁶;
m represents 1;
A represents piperidine or piperazine;
and R⁶, R^{b}, X, L and B have the meaning described above.

Another preferred embodiment of the present invention are the compounds of formula I wherein:
R¹ represents —SO₂R²;
R² represents aryl optionally substituted with one or more groups R^{b};
each R⁴ represents hydrogen;
R⁵ represents hydrogen;
W represents —CH₂—;
Z represents —CO—;
E represents —COOR⁶;
m represents 1;
A represents piperidine or piperazine;
L represents —(CH₂)ₙ—;
and R⁶, R^{b}, X, B and n have the meaning described above.

Another preferred embodiment of the present invention are the compounds of formula I wherein:
R¹ represents —SO₂R²;
R² represents aryl optionally substituted with one or more groups R^{b};
each R⁴ represents hydrogen;
R⁵ represents hydrogen;
W represents —CH₂—;
Z represents —CO—;
E represents —COOR⁶;
m represents 1;
A represents piperidine or piperazine;
L represents —(CH₂)ₙ—;
B represents Het¹ or Het², optionally substituted with one or more groups selected from oxo, R^{b} and Cy optionally substituted with one or more groups R^{b};
and R⁶, R^{b}, X, n, Cy, Het¹ and Het² have the meaning described above.

Another preferred embodiment of the present invention are the compounds of formula I wherein:
R¹ represents —SO₂R²;
R² represents aryl optionally substituted with one or more groups R^{b};
each R⁴ represents hydrogen;
R⁵ represents hydrogen;
W represents —CH₂—;
Z represents —CO—;
E represents —COOR⁶;
m represents 1;
A represents piperidine or piperazine;
L represents —(CH₂)ₙ—;
B represents —NR^{f}R^{f}, —OR^{f}, —NR^{f}COR^{f}, —NR^{f}CONR^{f}R^{f}, —NR^{f}CSNR^{f}R^{f}, —NR^{f}COOR^{e} or —OCONR^{f}R^{f};
and R⁶, R^{b}, R^{f}, R^{e}, X and n have the meaning described above.

Another preferred embodiment of the present invention are the compounds of formula I wherein:
R¹ represents —SO₂R²;
R² represents aryl optionally substituted with one or more groups R^{b};
each R⁴ represents hydrogen;
R⁵ represents hydrogen;
W represents —CH₂—;
Z represents —CO—;
E represents —COOH;
m represents 1;
X represents —NH—;
A represents piperidine or piperazine;
L represents —(CH₂)ₙ—;
B represents Het¹ or Het², optionally substituted with one or more groups selected from oxo, R^{b} and Cy optionally substituted with one or more groups R^{b};
and R^{b}, Cy, n, Het¹ and Het² have the meaning described above.

Another preferred embodiment of the present invention are the compounds of formula I wherein:

R¹ represents —SO₂R²;
R² represents aryl optionally substituted with one or more groups R^{b};
each R⁴ represents hydrogen;
R⁵ represents hydrogen;
W represents —CH₂—;
Z represents —CO—;
E represents —COOH;
m represents 1;
X represents —NH—;
A represents piperidine or piperazine;
L represents —(CH₂)ₙ—;
B represents —NR^{f}R^{f}, —OR^{f}, —NR^{f}COR^{f}, —NR^{f}CONR^{f}R^{f}, —NR^{f}CSNR^{f}R^{f}, —NR^{f}COOR^{e} or —OCONR^{f}R^{f};
and R^{b}, R^{f}, R^{e} and n have the meaning described above.

Another preferred embodiment of the present invention are the compounds of formula I wherein:
R¹ represents —SO₂R²;
R² represents aryl optionally substituted with one or more groups R^{b};
each R⁴ represents hydrogen;
R⁵ represents hydrogen;
W represents —CH₂—;
Z represents —CO—;
E represents —COOH;
m represents 1;
X represents —CH₂— or —O—;
A represents piperidine or piperazine;
L represents —(CH₂)ₙ—;
B represents Het¹ or Het² optionally substituted with one or more groups selected from oxo, R^{b} and Cy optionally substituted with one or more groups R^{b};
and R^{b}, Cy, n, Het¹ and Het² have the meaning described above.

Another preferred embodiment of the present invention are the compounds of formula I wherein:
R¹ represents —SO₂R²;
R² represents aryl optionally substituted with one or more groups R^{b};
each R⁴ represents hydrogen;
R⁵ represents hydrogen;
W represents —CH₂—;
Z represents —CO—;
E represents —COOH;
m represents 1;
X represents —CH₂— or —O—;
A represents piperidine or piperazine;
L represents —(CH₂)ₙ—;
B represents —NR^{f}R^{f}, —OR^{f}, —NR^{f}COR^{f}, —NR^{f}CONR^{f}R^{f}, —NR^{f}CSNR^{f}R^{f}, —NR^{f}COOR^{e} or —OCONR^{f}R^{f};
and R^{b}, R^{f}, R^{e} and n have the meaning described above.

Another preferred embodiment of the present invention are the compounds of formula I wherein:
R¹ represents —SO₂R²;
R² represents aryl optionally substituted with one or more groups R^{b};
each R⁴ represents hydrogen;
R⁵ represents hydrogen;
W represents —CH₂—;
Z represents —CO—;
E represents —COOH;
m represents 1;
X represents —NH—;
A represents piperidine or piperazine;
L represents —(CH₂)ₙ—;
B represents imidazopyridine optionally substituted with one or more groups selected from oxo, R^{b} and Cy optionally substituted with one or more groups R^{b};
and R^{b}, Cy and n have the meaning described above.

Another preferred embodiment of the present invention are the compounds of formula I wherein:
R¹ represents —SO₂R²;
R² represents aryl optionally substituted with one or more groups R^{b};
each R⁴ represents hydrogen;
R⁵ represents hydrogen;
W represents —CH₂—;
Z represents —CO—;
E represents —COOH;
m represents 1;
X represents —NH—;
A represents piperidine or piperazine;
L represents —(CH₂)ₙ—;
B represents —OCONR^{f}R^{f};
and R^{b}, R^{f} and n have the meaning described above.

Another preferred embodiment of the present invention are the compounds of formula I wherein:

R¹ represents —SO₂R²;
R² represents aryl optionally substituted with one or more groups R^{b};
each R⁴ represents hydrogen;
R⁵ represents hydrogen;
W represents —CH₂—;
Z represents —CO—;
E represents —COOH;
m represents 1;
X represents —CH₂— or —O—;
A represents piperidine or piperazine;
L represents —(CH₂)ₙ—;
B represents imidazopyridine optionally substituted with one or more groups selected from oxo, R^{b} and Cy optionally substituted with one or more groups R^{b};
and R^{b}, Cy and n have the meaning described above.

Another preferred embodiment of the present invention are the compounds of formula I wherein:
R¹ represents —SO₂R²;
R² represents aryl optionally substituted with one or more groups R^{b};
each R⁴ represents hydrogen;
R⁵ represents hydrogen;
W represents —CH₂—;
Z represents —CO—;
E represents —COOH;
m represents 1;
X represents —CH₂— or —O—;
A represents piperidine or piperazine;
L represents —(CH₂)ₙ—;
B represents —OCONR^{f}R^{f};
and R^{b}, R^{f} and n have the meaning described above.

Another preferred embodiment of the present invention are the compounds of formula I wherein:
R¹ represents —SO₂R²;
R² represents aryl optionally substituted with one or more groups R^{b};
each R⁴ represents hydrogen;
R⁵ represents hydrogen;
W represents —CH₂—;
Z represents —CO—;
E represents —COOH;
m represents 1;
X represents —NH—;
A represents piperidine or piperazine;
L represents —(CH₂)ₙ—;
B represents —OCONR^{f}R^{f}, wherein both groups R^{f} are attached to each other to form together with the nitrogen atom a cycle Het¹, which can be optionally substituted with one or more groups selected from oxo, Cy and R^{b}, wherein the groups Cy can be optionally substituted with one or more groups selected from oxo and R^{b};
and R^{b}, Cy, n and Het¹ have the meaning described above.

Another preferred embodiment of the present invention are the compounds of formula I wherein:
R¹ represents —SO₂R²;
R² represents aryl optionally substituted with one or more groups R^{b};
each R⁴ represents hydrogen;
R⁵ represents hydrogen;
W represents —CH₂—;
Z represents —CO—;
E represents —COOH;
m represents 1;
X represents —CH₂— or —O—;
A represents piperidine or piperazine;
L represents —(CH₂)ₙ—;
B represents —OCONR^{f}R^{f}, wherein both groups R^{f} are attached to each other to form together with the nitrogen atom a cycle Het¹, which can be optionally substituted with one or more groups selected from oxo, Cy and R^{b}, wherein the groups Cy can be optionally substituted with one or more groups selected from oxo and R^{b};
and R^{b}, Cy, n and Het¹ have the meaning described above.

The compounds of the present invention can contain one or more acid protons and one or more basic nitrogens and, consequently, can form salts with organic and inorganic bases and acids, which are also included in the present invention. There is no limitation on the nature of said salts, provided that, when used for therapeutic purposes, they are pharmaceutically acceptable. Examples of said salts include: salts with inorganic cations such as sodium, potassium, calcium, magnesium, lithium, aluminum, zinc, etc; salts formed with pharmaceutically acceptable amines such as ammonia, alkylamines, hydroxyalkylamines, lysine, arginine, N-methylglucamine, procaine and the like; salts with inorganic acids such as hydrochloric acid, hydrobromic acid, hydroiodic acid, nitric acid, perchloric acid, sulfuric acid or phosphoric acid; and salts with organic acids, such as methanesulfonic acid, trifluoromethanesulfonic acid, ethanesulfonic acid, benzenesulfonic acid, p-toluenesulfonic acid, fumaric acid, oxalic acid, acetic acid or maleic acid, among others. The salts can be prepared by treatment of a compound of formula I with a sufficient amount of the desired acid or base to give the salt in a conventional manner. The compounds of formula I and their salts differ in certain physical properties, such as solubility, but they are equivalent for the purposes of the invention.

Some compounds of the present invention can exist in solvated form, including hydrated forms. In general, the solvated forms, with pharmaceutically acceptable solvents such as water, ethanol and the like, are equivalent to the unsolvated form for the purposes of the invention.

The compounds of the present invention can exist as various diastereoisomers and/or various optical isomers. Diastereoisomers can be separated by conventional techniques such as chromatography or fractional crystallization. The optical isomers can be resolved using conventional techniques of optical resolution, to give the optically pure isomers. This resolution can be performed upon any chiral synthetic intermediate or upon the products of general formula I. The optically pure isomers can also be individually obtained using enantioespecific synthesis. The present invention covers both the individual isomers and the mixtures (for example racemic mixtures), whether obtained by synthesis or by physically mixing them up.

Furthermore, some of the compounds of the present invention can exhibit cis/trans isomery. The present invention includes each one of the geometric isomers as well as the mixtures thereof.

The compounds of the present invention can be prepared following the processes that are explained in detail below. As it will be obvious to a person skilled in the art, the precise method used for the preparation of a given compound can vary depending on its chemical structure. Furthermore, in most processes that are explained below it may be necessary or advisable to protect the labile or reactive groups using conventional protecting groups. Both the nature of said protecting groups and the processes for their introduction and removal are well known and belong to the state of the art (see for example Greene T. W. and Wuts P. G. M, “Protective Groups in Organic Synthesis”, John Wiley & Sons, 3^{rd} edition, 1999). By way of illustration, the groups tert-butoxycarbonyl (Boc), benzyloxycarbonyl (Cbz) or fluorenylmethoxycarbonyl (Fmoc) can be used as protecting groups for an amino function, or the triphenylmethyl group can be used as a protecting group for the acidic nitrogen of a tetrazole. Carboxyl groups can be protected, for example, as C₁₋₄ alkyl esters, such as methyl, ethyl or tert-butyl, or arylC₁₋₄ alkyl esters, such as benzyl. When a protecting group is present, a later deprotection step will be necessary, which is performed under standard conditions, such as those described in the reference above mentioned.

Given that the compounds of formula I contain several functional groups in their structure, their process of preparation will comprise the formation of said functional groups by the subsequent union of different building blocks. As it will be obvious to those skilled in the art, the order in which these reactions are carried out does not matter as long as the reactive or labile groups are protected, when necessary, with protecting groups.

A preferred method of synthesis for obtaining the compounds of formula I involves the formation, in a last step, of the —XC(O)A- group, wherein X and A have the meaning described above. This group can be prepared either by forming the bond between X and CO, or between CO and A, or by forming both bonds at the same time, depending on the meaning of X and A.

Thus, for example, the compounds of formula I wherein X is —NR⁵— (Ia) 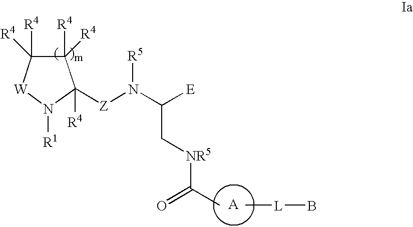
wherein R¹, R⁴, R⁵, W, Z, E, A, L, B and m have the meaning described above, can be obtained by formation of the amide group reacting an amine of formula II with an acid of formula III 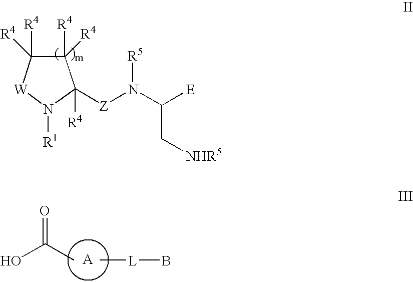
wherein R¹, R⁴, R⁵, W, Z, E, A, L, B and m have the meaning described above.

To prepare said amide, any conventional reaction of formation of amides can be used. For example, the carboxylic acid III can be reacted with the amine of formula II in the presence of a suitable condensing agent such as a carbodiimide (for example dicyclohexylcarbodiimide, N-(3-dimethylaminopropyl)-N′-ethylcarbodiimide, 1,3-diisopropylcarbodiimide, O-(benzotriazol-1-yl)-N,N,N′,N′-tetramethyluronium hexafluorophosphate (HBTU) or (benzotriazol-1-yloxy)tris(dimethylamino)phosphonium hexafluorophosphate (BOP)), alone or associated with 1-hydroxybenzotriazole, in a suitable solvent. Examples of suitable solvents include substituted amides, such as dimethylformamide; ethers, such as dioxane and tetrahydrofuran; and halogenated hydrocarbons, such as dichloromethane and chloroform. When the starting amine or the carbodiimide are used as an addition salt, for example the hydrochloride, the reaction is performed in the presence of a base, such as triethylamine, N,N-diisopropylethylamine or N-methylmorpholine.

Alternatively, the amide bond can be prepared by reacting the amine with a reactive derivative of the acid of formula III, such as its acid chloride, its N-hydroxysuccinimide ester, its anhydride or a mixed anhydride. In this case, the reaction is carried out in the presence of a proton scavenger base, for example pyridine, triethylamine or N,N-diisopropylethylamine, in a suitable solvent, or, when appropriate, the same proton scavenger amine can be used as solvent. Examples of suitable solvents include halogenated hydrocarbons, such as dichloromethane and chloroform; ethers, such as diethyl ether, tetrahydrofuran and dioxane; and aromatic hydrocarbons such as benzene and toluene.

The compounds of formula I wherein X is —O— (Ib) 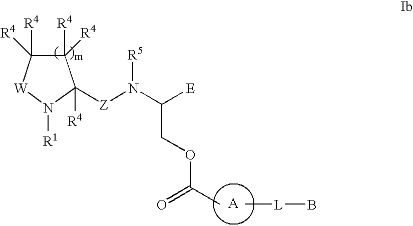
wherein R¹, R⁴, R⁵, W, Z, E, A, L, B and m have the meaning described above, can be obtained by formation of the ester bond reacting an alcohol of formula IV with an acid of formula III 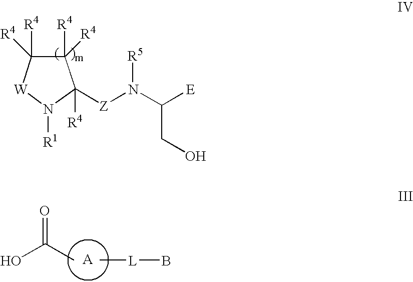
wherein R¹, R⁴, R⁵, W, Z, E, A, L, B and m have the meaning described above.

Any conventional method of preparation of esters can be used here, for example, a reactive derivative of the carboxylic acid III can be reacted with the alcohol of formula IV in the presence of a base and in a suitable solvent. Thus, for example, the reaction can be carried out using N,N′-dicyclohexylcarbodiimide as activating agent, in the presence of 4-dimethylaminopyridine and in a solvent such as diethyl ether. Other suitable reaction conditions include the use of bis(2-oxo-3-oxazolidinyl)phosphinic chloride (BOP-CI) and triethylamine in dichloromethane or the use of 2,4,6-trichlorobenzoyl chloride and triethylamine in tetrahydrofuran.

The compounds of formula I wherein X is —CH₂— and cycle A is bound to the carbonyl group through a nitrogen atom (Ic) 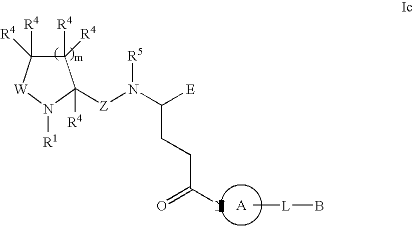
wherein R¹, R⁴, R⁵, W, Z, E, A, L, B and m have the meaning described above, can be obtained by reacting an acid of formula V with an amine of formula VI 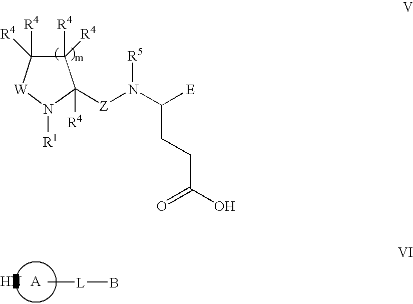
wherein R¹, R⁴, R⁵, W, Z, E, A, L, B and m have the meaning described above, using the methods described above for the formation of amide bonds.

The compounds wherein X is —NR⁵— or —O— and cycle A is bound to the carbonyl group through a nitrogen atom (Id and Ie, respectively) 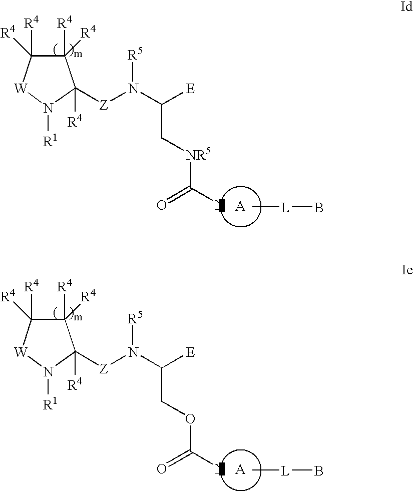
wherein R¹, R⁴, R⁵, W, Z, E, A, L, B and m have the meaning described above, can be obtained by forming the urea (compound Id) or carbamate (compound Ie) groups starting from the compounds of formulae II and VI in the first case and from the compounds of formulae IV and VI in the second one.

Said functions are prepared using a two-step procedure: in a first step, one of the amines II or VI in the case of the urea, or the alcohol of formula IV in the case of the carbamate, is reacted with an activating agent suitable for the preparation of ureas or carbamates such as triphosgene, phosgene or 1,1′-carbonyldiimidazole, in the presence of a base such as diisopropylethylamine, triethylamine or N-methylmorpholine, in a suitable solvent such as acetonitrile or a halogenated hydrocarbon such as chloroform or dichloromethane. The resulting compound is reacted in a second step with the second amine in the case of the urea and with the amine of formula VI in the case of the carbamate, in the presence of any of the bases cited above if the amine is present as hydrochloride, and in a suitable solvent, for example the solvent used in the first step. The reaction can be carried out at a temperature comprised between room temperature and the boiling point of the solvent.

The activation of the primary amine of formula II or of the alcohol of formula IV in the first step can also be carried out by reacting said compounds with agents such as phenyl chloroformate or nitrophenyl chloroformate, under the same conditions described above for the other activating agents.

The ureas of formula Id can also be obtained by reaction of an amine of formula II with di-tert-butyl dicarbonate as activating agent, in the presence of a catalytic amount of 4-dimethylaminopyridine (DMAP) and then reacting the resulting intermediate with the amine of formula VI. Both steps are carried out in a suitable solvent such as dichloromethane, and it is not necessary to isolate the obtained intermediate.

Alternatively, the ureas of formula Id can also be obtained in two steps from a compound of formula V′ 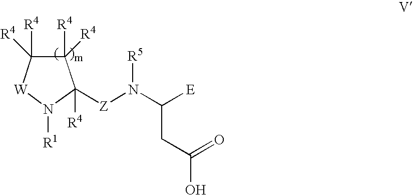
wherein R¹, R⁴, R⁵, W, Z, E and m have the meaning described above. In a first step, a Curtius rearrangement is carried out, that is, the compound of formula V′ is treated with a suitable azide for carrying out said rearrangement such as for example sodium azide combined with thionyl chloride, or diphenylphosphorylazide, in the presence of a base such as triethylamine, diisopropylethylamine or N-methylmorpholine. The reaction is preferably carried out in an apolar solvent such as benzene, stirring first at room temperature and then at a temperature comprised between room temperature and reflux. The resulting compound is reacted in a second step with an amine of formula VI, in an inert solvent such as for example a halogenated hydrocarbon such as dichloromethane or chloroform, or a substituted amide such as dimethylformamide, optionally in the presence of a base such as diisopropylethylamine, triethylamine or pyridine. The reaction can be carried out at a temperature comprised between room temperature and reflux, optionally irradiating with microwaves, when necessary.

The intermediates of formulae II, IV, V and V′ used in the reactions described above can be obtained, when Z is a carbonyl group (compounds of formulae II*, IV*, V* and V′*), by reacting the acid of formula VIII with the amines of formulae IIa, IVa, Va or V′a as shown in the following scheme 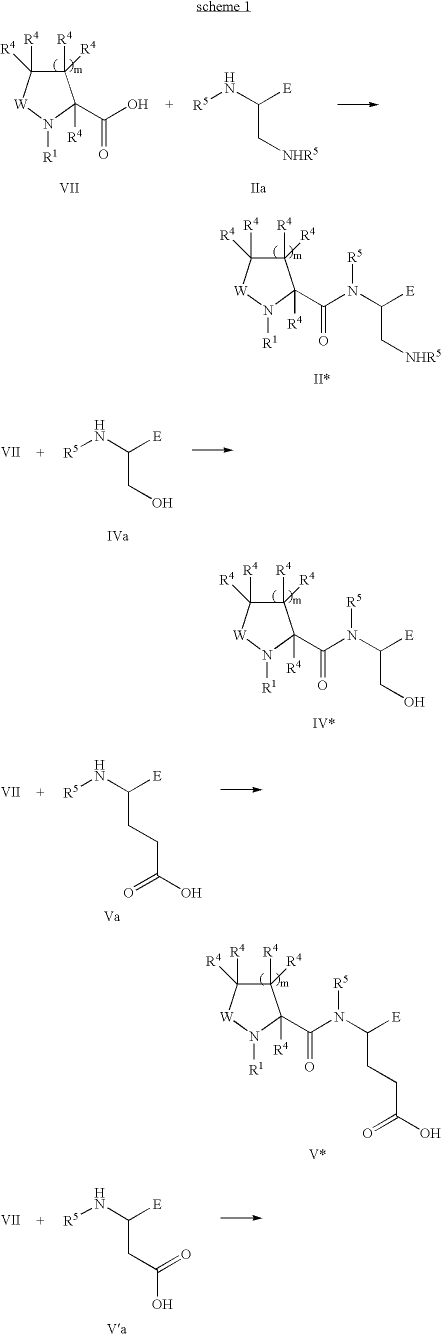 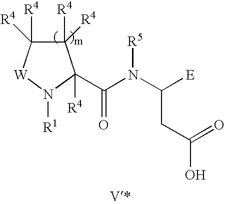
wherein R¹, R⁴, R⁵, W, E and m have the meaning described above. Said reactions are carried out according to the methods described above for the preparation of amide bonds, previously protecting, if necessary, the labile or reactive groups with conventional protecting groups. The compounds of formulae II, IV, V and V′ wherein Z represents —CS— can be obtained from the compounds II*, IV*, V* and V′* respectively, by transforming the amide group into a thioamide, if no other amide group is present in the molecule. Said transformation can be carried out using any of the methods widely described in the literature, for example, by treatment with phosphorous pentasulfide or Lawesson's reagent in a suitable solvent such as tetrahydrofuran or toluene, or with oxalyl chloride and trimethylsilyl sulfide (TMS₂S), triphosgene/TMS₂S or POCl₃/TMS₂S in dichloromethane.

The precursors of formula VII can be commercially available or can be obtained from commercially available compounds by reactions widely known by any person skilled in the art.

Thus, for example, the compounds of formula VII wherein R¹ represents —SO₂R₂ (VIIa) 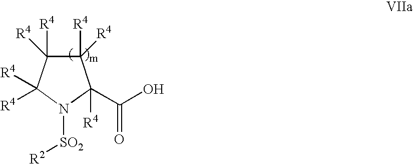
wherein R², R⁴ and m have the meaning described above, can be obtained by formation of the sulfonamide from an amine of formula VIIIa and a sulfonyl chloride of formula IX 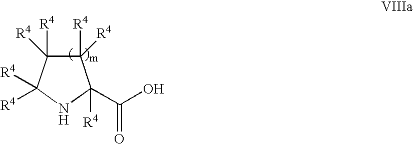
wherein R², R⁴ and m have the meaning described above. The reaction can be carried out in basic medium, using a base such as sodium carbonate, sodium hydroxide or triethylamine, and in a suitable solvent such as an ether, for example dioxane, or a halogenated hydrocarbon, for example chloroform or dichloromethane.

The compounds of formula VII wherein R¹ represents —COR² (VIIb) 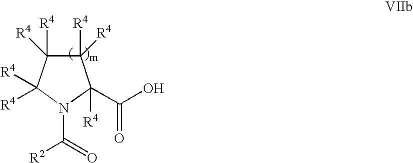
wherein R², R⁴ and m have the meaning described above, can be obtained by formation of the amide group according to the methods described above, starting from an amine of formula VIIIa having the carboxy group unprotected or protected as an ester, and an acid of formula R²COOH (X) or an activated form thereof.

Finally, the compounds of formula VII wherein R¹ represents —CH₂R³ (VIIc) 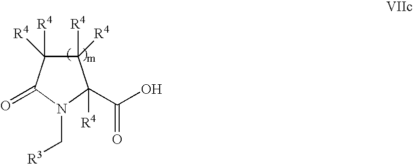
wherein R³, R⁴ and m have the meaning described above, can be obtained by alkylation of the amide of formula VIIIb 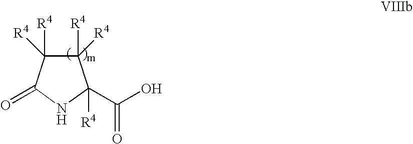
wherein R⁴ and m have the meaning described above, with a compound of formula R³—CH₂-D (XI), wherein R³ has the meaning described above and D is a good leaving group, for example an alkylsulfonate or arylsulfonate such as mesylate or tosylate, or a halogen such as Cl, Br or I. This reaction is carried out under the standard conditions for the alkylation of amides, that is in the presence of a strong base such as sodium hydride in a suitable solvent and at a temperature comprised between room temperature and the boiling point of the solvent. Examples of solvents include halogenated hydrocarbons such as dichloromethane or chloroform, substituted amides such as for example dimethylformamide and alcohols such as ethanol.

The intermediates of formulae III and VI used in the reactions described above 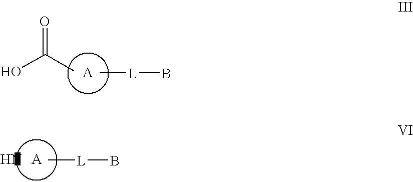
wherein A, L and B have the meaning described above, can be obtained by reactions widely known by any person skilled in the art, using one or another depending on the specific structure sought.

Thus, for example, the compounds of formulae III and VI wherein cycle A is bound to the -L-B moiety through a ring nitrogen atom (compounds of formulae IIIa and VIa, respectively) 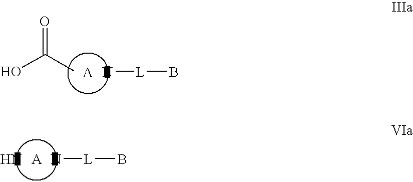
wherein A, L and B have the meaning described above, can be obtained by alkylation of the amine of cycle A, by reacting the compounds of formulae XII and XIII, respectively, 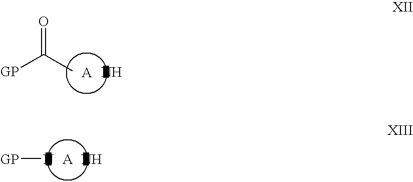
wherein A has the meaning described above and GP is a suitable protecting group, with a compound of formula D-L-B (XIV) wherein D, L and B have the meaning described above, and subsequent removal of the protecting group. The reaction of XII or XIII with XIV is carried out under the standard conditions for the alkylation of amines, that is in the presence of a base such as triethylamine, sodium carbonate, potassium carbonate or sodium hydride in a suitable solvent such as a halogenated hydrocarbon (for example dichloromethane or chloroform), a substituted amide (for example dimethylformamide) or an alcohol (for example ethanol or butanol), and at a temperature comprised between room temperature and the boiling point of the solvent.

The compounds of formulae III and VI can also be obtained by forming the bond between the groups L and B. When B is a cycle Het¹ or Het² bound to the rest of the molecule through a ring nitrogen atom (IIIb and VIb) 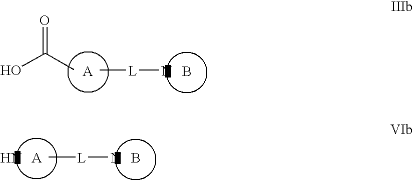
wherein A and L have the meaning described above, the compounds of formulae IIIb and VIb can be obtained by alkylation of the nitrogen of the amine of cycle B (XV) with a compound of formula XIIa or XIIIa respectively, 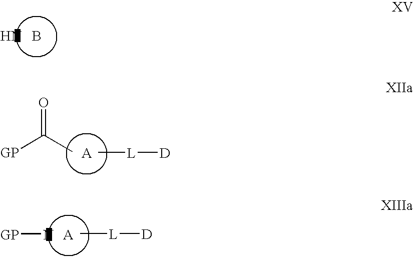
wherein A, L, B, D and GP have the meaning described above, under the conditions described above for the alkylation of amines, and by subsequent removal of the protecting group.

Alternatively, the compounds of formulae IIIb and VIb can also be obtained by construction of the cycle B starting from a primary amine of formula XIIb or XIIIb 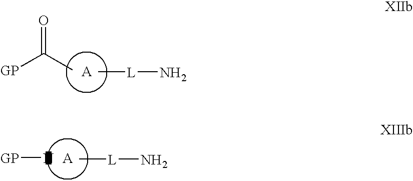
wherein A, L and GP have the meaning described above, by reaction them with a suitable bifunctional compound, and subsequent removal of the protecting group GP. By way of illustration, some suitable reactions of construction of the cycle B are shown below. 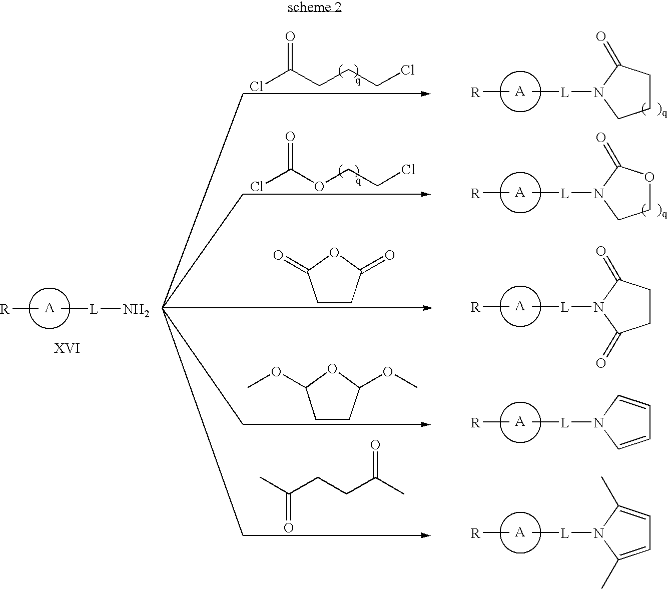
wherein the compound XVI represents in combined form the compounds XIIb and XIIIb, A and L have the meaning described above, q represents 0, 1 or 2 and R represents GP-CO— or

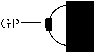

When B represents one of the functions ii), that is —COR^{e}, —NR^{f}R^{f}, —OR^{f}, —SR^{f}, —S(O)ₚR^{e}, —CONR^{f}R^{f}, —NR^{f}COR^{f}, —NR^{f}CONR^{f}R^{f}, —NR^{f}CSNR^{f}R^{f}, —NR^{f}COOR^{e}, —OCOR^{e}, —OCONR^{f}R^{f}, —NR^{f}SO₂R^{e} or —SO₂NR^{f}R^{f}, the compounds of formulae III and VI can be obtained by forming said functions starting from suitable precursors, using standard reactions in organic chemistry such as those explained below. Though not being mentioned each time, whenever in said reactions a protecting group GP is present in the starting products, a final deprotection step will be needed to provide the compounds of formulae III and VI.

Thus, for example, the compounds of formulae III and VI wherein B is —NHCOR^{e} and —NHSO₂R^{e} can be obtained from the compounds of formulae XIIb and XIIIb by forming the amide and sulfonamide groups, reacting them with an acid R^{e}COOH or a sulfonyl chloride R^{e}SO₂Cl, respectively, under the conditions described above for the preparation of amides and sulfonamides. The compounds of formulae III and VI wherein B represents an urea, thiourea or carbamate function can also be obtained from a compound of formula XVI. Thus, the compounds of formulae III and VI wherein B is —NHCONR^{f}R^{f} can be obtained, for example, by using the method for preparing ureas described above; the compounds of formulae III and VI wherein B is —NHCSNR^{f}R^{f} can be obtained following the same method but using thiophosgene instead of triphosgene as coupling agent. The compounds of formulae III and VI wherein B represents an urea or thiourea of formula —NHCONHR^{e} or —NHCSNHR^{e} can be obtained by reaction of an amine of formula XVI with an isocyanate of formula R^{e}NCO or a thioisocyanate of formula R^{e}NCS, respectively. This reaction is carried out by reacting the amine XVI with the desired isocyanate or thioisocyanate in an inert solvent such as for example toluene, a substituted amide such as dimethylformamide or an ether such as tetrahydrofuran. The compounds of formula III and VI wherein B is a carbamate of formula —NHCOOR^{e} can be obtained by reaction of an amine XVI with a chloroformate of formula ClCOOR^{e}, carrying out the reaction in the presence of a base such as a tertiary amine (triethylamine, diisopropylethylamine or N-methylmorpholine) and in a suitable solvent such as for example a halogenated hydrocarbon such as chloroform or dichloromethane. Finally, a compound of formula XVI can also be transformed into a secondary or tertiary amine (that is, a compound of formula III or VI wherein the group B is —NR^{f}R^{e}) by alkylation with one or two compounds of formula D-R^{e}, wherein D represents a good leaving group, using the methods described above or alternatively by reductive amination of a suitable aldehyde or ketone. This reaction is in general carried out by reacting the amine with an aldehyde or ketone in the presence of a suitable reducing agent such as a metallic hydride, for example sodium cyanoborohydride or sodium triacetoxyborohydride, and in a suitable solvent such as methanol, tetrahydrofuran, acetonitrile or mixtures thereof, among others.

The compounds of formulae III and VI wherein B is —OCOR^{e}, —OCONR^{f}R^{f}, or —OR^{f} can be obtained from a compound of formula XVIa 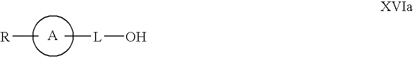
wherein R, A and L have the meaning described above. Thus, starting from said compound XVIa, the compounds III and VI that contain ester and carbamate groups can be easily obtained by using the methods described above for the preparation of said functions. The compounds of formulae III and VI wherein B is an ether of formula —OR^{e} can be obtained for example by using the Mitsunobu reaction, reacting a compound of formula XVIa with another alcohol of formula R^{e}OH in the presence of triphenylphosphine and diethyl azodicarboxylate (DEAD), in a suitable solvent such as tetrahydrofuran. The formation of the ether can also be carried out by reacting the alcohol of formula XVIa with a suitable base such as potassium carbonate in a solvent such as acetone or 2-butanone and treating the obtained salt with a compound of formula R^{e}-D, wherein D represents a good leaving group. The compounds of formulae III and VI wherein B is —OR^{f} and R^{f} is hydrogen are obtained from an alcohol of formula XVIa by deprotection.

The compounds of formulae III and VI wherein B is —SR^{f} or —S(O)ₚR^{e} can be in general obtained from a compound of formula XVIb 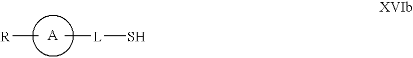
wherein R, A and L have the meaning described above. Thus, for example, the compounds of formulae III and VI wherein B is a sulfide of formula —SR^{e} can be obtained by reacting the thiol XVIb with an akylating agent R^{e}-D, wherein D is preferably a tosylate, in the presence of a base such as 1,8-diazabicyclo[5.4.0]undec-7-ene (DBU) and in a suitable solvent such as N,N-dimethylformamide and subsequent removal of the protecting group. The compounds of formulae III and VI wherein B is —SR^{f} and R^{f} is hydrogen are directly obtained from the thiol of formula XVIb by deprotection. The compounds of formulae III and VI wherein B is —S(O)ₚR^{e} can be obtained by oxidation of the previously obtained sulfide (III or VI wherein B is —SR^{e}) using a suitable oxidizing agent. Suitable reagents for the oxidation of a sulfide to a sulfoxide (—SOR^{e}) include among others hydrogen peroxide; meta-chloroperbenzoic acid and sodium periodate. The oxidation reaction is carried out reacting the corresponding sulfide with one equivalent of the oxidizing agent in a suitable solvent such as for example dichloromethane. Likewise, the compounds III and VI wherein B is —SO₂R^{e} can be obtained either from the corresponding sulfide by reaction with at least two equivalents of a suitable oxidizing agent, such as hydrogen peroxide, sodium tungstate, meta-chloroperbenzoic acid or potassium permanganate, or from the corresponding sulfoxide by reaction with at least one equivalent of the oxidizing agent.

The compounds of formulae III and VI wherein B is a sulfonamide (—SO₂NR^{f}R^{f}), can be obtained, for example, by a sequence that comprises the conversion of a compound of formula XIIa or XIIIa, respectively, 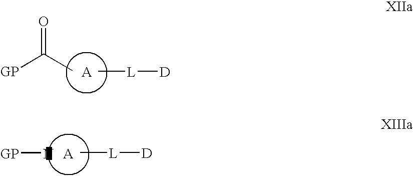
wherein A, L, D and GP have the meaning described above, into the corresponding thioacetate (—SAc), the subsequent transformation of said thioacetate into the corresponding sulfonyl chloride by treatment with chlorine and the reaction of said sulfonyl chloride with an amine HNR^{f}R^{f}, following a sequence analogous to that described by R. J. Watson et al. in Tetrahedron Letters, 2002, 43, 683-685, followed by the removal of the protecting group GP.

The precursors of formulae XII, XIII, XIIa, XIIIa, XIIb, XIIIb, XVIa and XVIb, which contain functional groups suitably protected, can be obtained by protection of commercially available compounds or compounds obtained from commercially available compounds by standard processes.

The precursors of formulae IIa, IVa, Va, V′a, VIIIa, VIIIb, IX, X, XI, XIV, XV, R^{e}COOH, R^{e}SO₂Cl, R^{e}NCO, R^{e}NCS, ClCOOR^{e}, R^{e}OH, HNR^{f}R^{f} and D-R^{e} can be commercially available or can be obtained from commercially available compounds using processes widely known by those skilled in the art.

By way of illustration, the compounds of formulae IIa, IVa, Va and V′a wherein E is an ester or an amide can be commercially available or can be obtained from the commercially available aminoacid (2,3-diaminopropionic acid, serine, glutamic and aspartic acid, respectively) by transforming the carboxylic acid present in all of them into an ester or amide group following the methods described above. The compounds of formulae IIa, IVa, Va and V′a wherein E is a 5-tetrazolyl group can be obtained in 2 steps from the primary amide of the corresponding aminoacid. Said amide is first transformed into the nitrile by treatment with an efficient dehydrating agent such as phosphorous pentoxide, phosphoryl chloride, thionyl chloride or acetic anhydride in a suitable solvent and the cyano group obtained is subsequently converted into a tetrazole by treatment with one equivalent of an azide such as tributyltin azide (previously formed or formed in situ from sodium azide and tributyltin chloride) in an apolar solvent such as xylene or toluene at a temperature comprised between room temperature and the boiling point of the solvent.

Moreover, the compounds of formula VIIIb can be commercially available or can be obtained by dehydrating a compound of formula VIII* 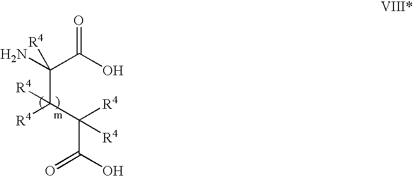
wherein m and R⁴ have the meaning described above, that is, by dehydrating aspartic and glutamic acids, when all the groups R⁴ represent hydrogen and m represents 0 and 1, respectively, or by dehydrating substituted derivatives thereof, when one or more groups R⁴ is different from hydrogen.

The compounds of formula I wherein Z represents CO (If) 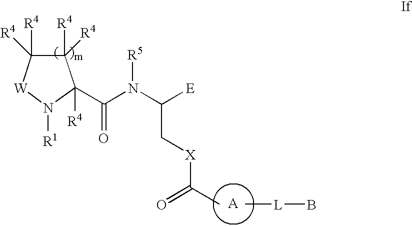
wherein R¹, R⁴, R⁵, W, E, X, A, L, B and m have the meaning described above, can also be obtained by forming the amido bond present in the molecule as the last step of the synthesis, starting from the intermediates of formula VII and XVII 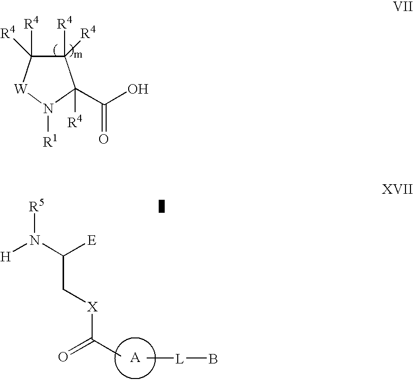
wherein R¹, R⁴R⁵, W, E, X, A, L, B and m have the meaning described above, using any of the methods described above for the formation of said amido bond.

The compounds of formula I wherein W represents —CR⁴R⁴— and R¹ represents —SO₂R² or —COR² (compounds of formulae Ig and Ih, respectively) 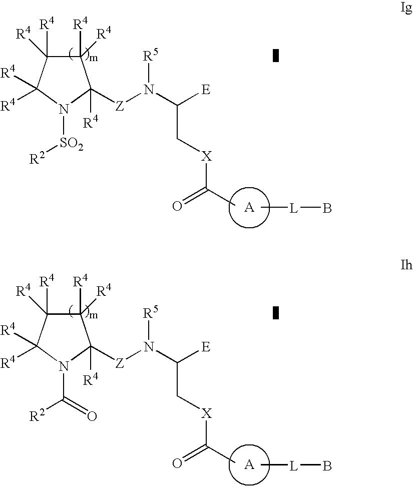
wherein R², R⁴, R⁵, Z, E, X, A, L, B and m have the meaning described above, can be obtained by forming, in a last step, the bond between the group R¹ and the compound of formula XVIII 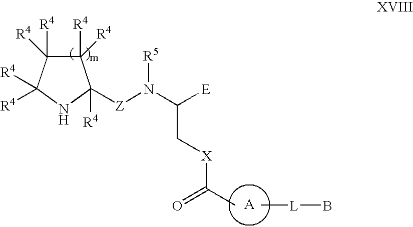
wherein R⁴, R⁵, Z, E, X, A, L, B and m have the meaning described above. Thus, depending on the meaning of the group R¹, the compound of formula XVIII can be reacted with a sulfonyl chloride of formula IX, or with an acid of formula X or an activated form thereof, to obtain a sulfonamide or amide, respectively. These reactions can be carried out under the conditions described above for the preparation of the precursors of formulae VIIa and VIIb, respectively.

The compounds of formula I wherein W represents —CO— and R¹ represents —CH₂R³ (Ii) 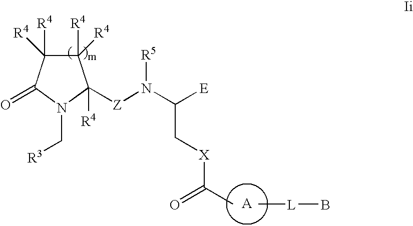
wherein R³R⁴, R⁵, Z, E, X, A, L, B and m have the meaning described above, can be obtained by alkylation of a compound of formula XIX 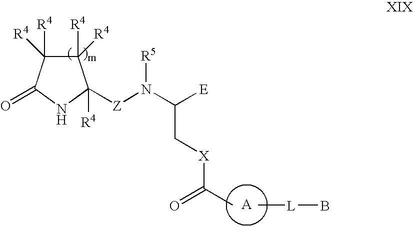
wherein R⁴, R⁵, Z, E, X, A, L, B and m have the meaning described above, with a compound of formula XI, under the standard conditions for the alkylation of amides described above in connection with compounds of formula VIIc.

The compounds of formula I wherein cycle A is bound to the -L-B moiety through a ring nitrogen atom (Ij) 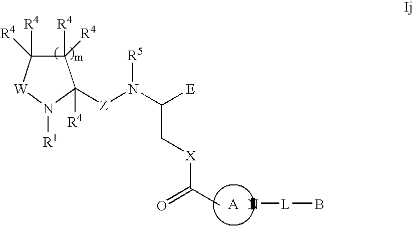
wherein R¹, R⁴, R⁵, W, Z, E, X, A, L, B and m have the meaning described above, can be obtained by alkylation of the amine of cycle A of a compound of formula XX 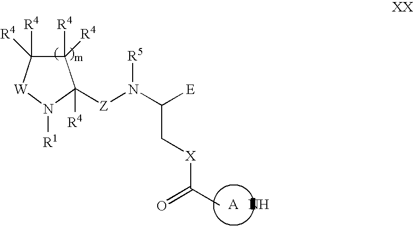
wherein R¹, R⁴, R⁵, W, Z, E, X, A and m have the meaning described above, with a compound of formula XIV, under the standard alkylation conditions described above.

The intermediates of formulae XVII, XVIII, XIX and XX can be obtained, as described for the compounds of formula I, by formation of the various functional groups present in said compounds, by the subsequent combination of different building blocks and/or the transformation of functional groups already present in said building blocks. All these reactions, as well as the reactions for preparing non-commercially available precursors, can be carried out by using the chemistry described throughout the present invention and by using standard reactions in organic chemistry. As in the case of the compounds of formula I, the order in which the reactions are carried out does not matter as long as the reactive or labile groups are protected, whenever necessary, with suitable protecting groups.

By way of illustration, the intermediates of formula XVII can be obtained by forming, in a last step, the —XC(O)A- group. The intermediates of formula XVII wherein X is —CH₂— and cycle A is bound to the carbonyl group through a ring carbon atom (VIIa) 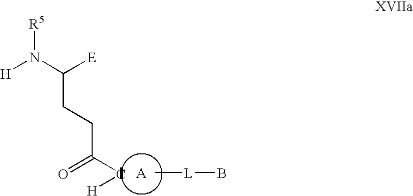
wherein R⁵, A, L, E and B have the meaning described above, may be obtained by following a method analogous to that described by Ying-zi Xu et al. in J. Org. Chem., 1999, 64, 4069-4078, by reaction of the compounds of formulae XXI and XXII as shown in the following scheme 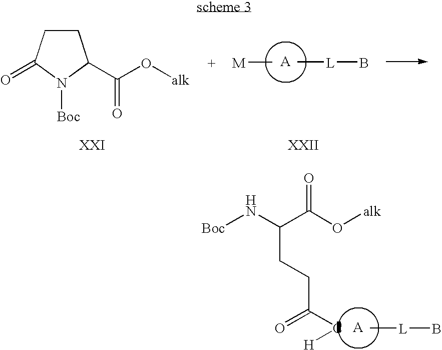
wherein A, L and B have the meaning described above, alk represents C₁₋₈ alkyl and M represents Li, Br—Mg—, Cl—Mg— or I—Mg—, and subsequent transformation of the compound obtained into a compound of formula XVIIa using standard reactions in organic chemistry. As it will be obvious to those skilled in the art, the compounds of formula XXI can be obtained by intramolecular cyclation of the corresponding glutamic acid derivative; the compounds of formula XXII can be obtained using standard methods for preparing organolithium or organomagnesium compounds, starting from the corresponding halide.

The compounds of the present invention can also be obtained by interconversion from another compound of formula I, in one or a plurality of steps, using standard reactions, widely used in organic chemistry.

For example, a group E can be converted into another group E, by transforming a carboxylic acid into an ester or amide as described above. Furthermore, the carboxylic acid can be obtained from the corresponding ester or amide by hydrolysis. The hydrolysis of an ester group to give a carboxy group can be carried out in the presence of a base such as potassium hydroxide or lithium hydroxide in a suitable solvent such as for example ethanol, tetrahydrofuran, ethanol-water mixtures and tetrahydrofuran-water mixtures or in an apolar solvent such as benzene in the presence of a crown ether, for example 18-C-6. The hydrolysis of the amide can be carried out for example by using a strong acid such as hydrochloric, hydrobromic, sulfuric or phosphoric acid either in a polar solvent such as water or ethanol-water mixtures, or in a basic medium by using a strong base such as sodium hydroxide or potassium hydroxide in ethylene glycol. Alternatively, a primary amide can be transformed into a tetrazolyl group as described above.

A group B can also be interconverted into another group B, giving thus rise to further compounds of formula I.

Thus, for example, the compounds of formula I wherein B represents a cycle Het¹ or Het² bound to the rest of the molecule through a ring nitrogen atom (Ik), 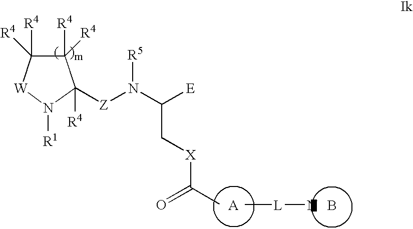
wherein R¹, R⁴, R⁵, W, Z, E, X, A, L, B and m have the meaning described above, can be obtained by construction of the cycle B upon a compound of formula Im 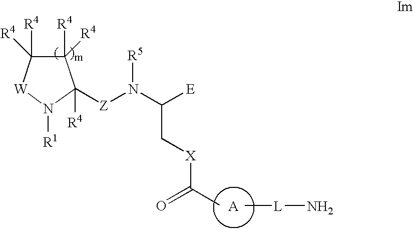
wherein R¹, R⁴, R⁵, W, Z, E, X, A, L and m have the meaning described above, by reaction with a suitable bifunctional compound such as those shown in scheme 2 above.

Moreover, the compounds of formula I wherein B represents a function of formula —NHCOR^{e}, —NHSO₂R^{e}, —NHCONR^{f}R^{f}, —NHCSNR^{f}R^{f}, —NHCONH^{e}, —NHCSNHR^{e}, —NHCOOR^{e} or —NR^{f}R^{e} can all be obtained from a primary amine of formula Im, by treatment with suitable reagents. Said reagents, as well as the reaction conditions, are the same as those described above for the preparation of compounds of formulae III and VI wherein B has the same meaning. Thus, the compounds wherein B represents —NHCOR^{e} can be obtained by reaction of an amine of formula Im with an acid R^{e}COOH or an activated form thereof; the compounds wherein B is —NHSO₂R^{e}, by reaction of the amine Im with a sulfonyl chloride of formula R^{e}SO₂Cl; the compounds wherein B is —NHCONR^{f}R^{f} or —NHCSNR^{f}R^{f}, by reaction of an amine Im with an amine of formula HNR^{f}R^{f}, where one of the two amines is activated with an activating agent suitable for the formation of ureas or thioureas; the compounds wherein B is —NHCONHR^{e} or —NHCSNHR^{e}, by reaction of an amine Im with an isocyanate of formula R^{e}NCO or with a thioisocyanate of formula R^{e}NCS, respectively; the compounds wherein B is —NHCOOR^{e}, by reaction of an amine Im with a chloroformate of formula ClCOOR^{e}; and the compounds wherein B is —NR^{f}R^{e}, by alkylation of an amine of formula Im with one or two compounds of formula D-R^{e} wherein D represents a good leaving group, or by reductive amination of a suitable aldehyde or ketone.

The compounds of formula I wherein B represents a function of formula —OCOR^{e}, —OCONR^{f}R^{f} or —OR^{f} can all be obtained from an alcohol of formula In 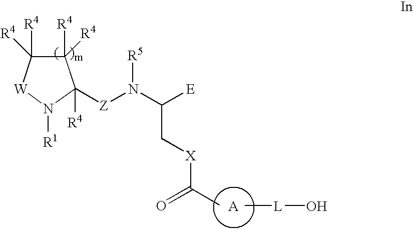
wherein R¹, R⁴, R⁵, W, Z, E, X, A, L and m have the meaning described above, by treatment with suitable reagents for the preparation of the desired function. Thus, when B represents —OCOR^{e}, an alcohol of formula In can be reacted with a reactive derivative of a carboxylic acid of formula R^{e}COOH, under the conditions described above for the formation of ester groups. For the preparation of compounds of formula I wherein B represents —OCONR^{f}R^{f}, an alcohol of formula In can be reacted with an amine of formula HNR^{f}R^{f} following any of the methods described above for the preparation of carbamates. The compounds wherein B represents an ether —OR^{e} can be obtained by reaction of an alcohol of formula In with an alcohol of formula R^{e}OH, or with an alkylating agent of formula D-R^{e}, wherein D has the meaning described above, according to the methods described above for the preparation of compounds of formulae III and VI wherein B is an ether-OR^{e}.

Likewise, the compounds of formula In can also give rise to further compounds of formula Ik by conversion into an intermediate of formula XXIII 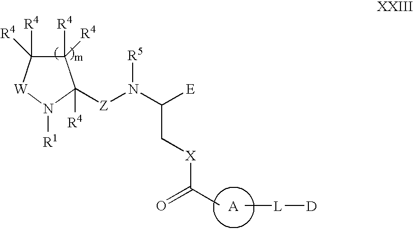
wherein R¹, R⁴, R⁵, W, Z, E, X, A, L, D and m have the meaning described above, and subsequent reaction of XXIII with an amine of formula XV, under the standard conditions for the alkylation of amines described above. The transformation of In into XXIII is performed by using standard reactions for transforming alcohols into leaving groups such as halides or alkyl or arylsulfonates.

Moreover, the intermediate of formula XXIII can be transformed into a compound of formula I wherein B represents —SO₂NR^{f}R^{f} by following a sequence analogous to that described by R. J. Watson et al. in Tetrahedron Letters, 2002, 43, 683-685.

The compounds of formula I wherein B is —SR^{f} or —S(O)ₚR^{e} can all be obtained from a compound of formula Io 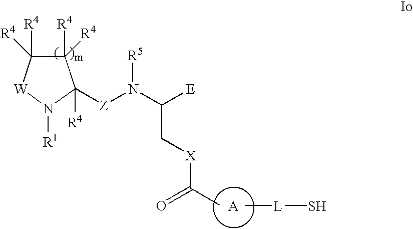
wherein R¹, R⁴, R⁵, W, Z, E, X, A, L and m have the meaning described above. Following the method described above for the preparation of compounds of formulae III and VI wherein B represents the same functions, a compound of formula Io can be transformed into a compound of formula I wherein B is —SR^{e}, by reaction of compound Io with an alkylating agent; the resulting sulfide (—SR^{e}) can then be oxidized to give compounds of formula I wherein B represents —SORE or —SO₂R^{e}.

Other transformations of groups that may be present in the compounds of formula I and that give rise to other compounds of formula I include, among others: the conversion of an amide into a thioamide, for example under the conditions described above; the conversion of a nitro group into an amino group, for example by hydrogenation in the presence of a suitable catalyst such as Pd/C or by treatment with a suitable reducing agent such as SnCl₂; and the conversion of a primary or secondary hydroxyl group into an amino, tioether or halogen group in a two-step procedure. First, the hydroxyl is converted into a good leaving group by treatment, for example, with a sulfonyl halide such as tosyl chloride in pyridine. The resulting tosylate can be easily converted into the corresponding azide by treatment with sodium azide, in a suitable solvent such as N,N-dimethylformamide-water mixtures, which can then be hydrogenated in the presence of a suitable catalyst such as Pd/C to give the corresponding amine. Likewise, said tosylate can be treated with a thiol in the presence of a base such as DBU, in a suitable solvent such as N,N-dimethylformamide to give the corresponding thioether. Moreover, the resulting tosylate can be treated for example with sodium iodide in acetone, lithium chloride in N,N-dimethylformamide or ethanol, or sodium bromide in N,N-dimethylformamide or dimethylsulfoxide, to give the corresponding halide.

The salts of the compounds of formula I can be prepared by conventional methods by treatment for example with an acid such as hydrochloric acid, sulfuric acid, nitric acid, oxalic acid or methanesulfonic acid, or by treatment with a base such as sodium hydroxide or potassium hydroxide.

As mentioned above, the compounds of the present invention act as antagonists of integrins α₄, which are involved in numerous cell adhesion pathological processes. Therefore, the compounds of the present invention are useful for the treatment or prevention of diseases mediated by integrins α₄. In a preferred embodiment, the compounds of the present invention are useful for the treatment or prevention of inflammatory, immune and/or autoimmune diseases selected from: diseases with an allergic component, such as for example asthma, allergic rhinitis, allergic dermatitis and allergic conjunctivitis; inflammatory diseases with an autoimmune component, such as for example rheumatoid arthritis, psoriatic arthritis, multiple sclerosis, psoriasis, and diabetes; inflammatory bowel disease, including Crohn's disease and ulcerative colitis; inflammatory processes having an alloimmune origin caused by transplants or rejections; inflammatory processes that develop as a consequence of blood vessel revascularization treatments, such as percutaneous transluminal coronary angioplasty; as well as other inflammatory diseases such as encephalomyelitis; hepatitis, bronchitis, vasculitis and atherosclerosis.

The compounds of the present invention can also be useful for the treatment of other disorders mediated by integrins α₄. For example, the compounds of formula I can inhibit cell proliferation and might therefore be useful for the treatment or prevention of tumor metastasis. Other uses of the compounds of formula I include the treatment or prevention of degenerative diseases, such as for example Alzheimer's disease and arthrosis, and the treatment or prevention of ischemia-reperfusion disorders, including among others acute coronary diseases and stroke.

According to the activity of the products herein described, the present invention also relates to compositions which contain a compound of the present invention, together with an excipient or other auxiliary agents if necessary. The compounds of the present invention can be administered in the form of any pharmaceutical formulation, the nature of which, as it is well known, will depend upon the nature of the active compound and its route of administration. Any route of administration may be used, for example oral, parenteral, nasal, ocular, rectal and topical administration.

According to the present invention, solid compositions for oral administration include tablets, granulates and capsules. In any case the manufacturing method is based on a simple mixture, dry granulation or wet granulation of the active compound with excipients. These excipients can be, for example, diluents such as lactose, microcrystalline cellulose, mannitol or calcium hydrogenphosphate; binding agents such as for example starch, gelatin or polyvinylpyrrolidone; disintegrants such as sodium carboxymethyl starch or sodium croscarmellose; and lubricating agents such as for example magnesium stearate, stearic acid or talc. Tablets can be additionally coated with suitable excipients by using known techniques with the purpose of delaying their disintegration and absorption in the gastrointestinal tract and thereby provide a sustained action over a longer period, or simply to improve their organoleptic properties or their stability. The active compound can also be incorporated by coating onto inert pellets using natural or synthetic film-coating agents. Soft gelatin capsules are also possible, in which the active compound is mixed with water or an oily medium, for example coconut oil, liquid paraffin or olive oil.

Powders and granulates for the preparation of oral suspensions by the additon of water can be obtained by mixing the active compound with dispersing or wetting agents; suspending agents and preservatives. Other excipients can also be added, for example sweetening, flavouring and colouring agents.

Liquid forms for oral administration include emulsions, solutions, suspensions, syrups and elixirs containing commonly-used inert diluents, such as distilled water, ethanol, sorbitol, glycerol, polyethylene glycols and propylene glycol. Said compositions can also contain coadjuvants such as wetting, suspending, sweetening, flavouring, preserving agents and buffers.

Injectable preparations, according to the present invention, for parenteral administration, comprise sterile solutions, suspensions or emulsions, in an aqueous or non-aqueous solvent such as propylene glycol, polyethylene glycol or vegetable oils. These compositions can also contain coadjuvants, such as wetting, preserving, emulsifying and dispersing agents. They may be sterilized by any known method or prepared as sterile solid compositions which will be dissolved in water or any other sterile injectable medium immediately before use. It is also possible to start from sterile materials and keep them under these conditions throughout all the manufacturing process.

The compounds of the present invention can also be formulated as a solid form, dissolved or dispersed in a suitable vehicle, for inhalation in single or multidose container. Preparations to be administered as an aerosol (dispersion of solid or liquid particles in a gas) use suitable devices such as nebulisers, pressured metered-dose inhalers or dry-powder inhalers. Depending on this, the compound will be formulated with excipients such as propellants responsible for developing the proper pressure within the container to force the content out through the opening of the valve, solvents, emulsifying agents, viscosity-increasing agents, preservatives, stabilizing agents and lubricants to avoid the blockade of the valve.

For the rectal administration, the active compound can be preferably formulated as a suppository on an oily base, such as for example vegetable oils or solid semisynthetic glycerides, or on a hydrophilic base such as polyethylene glycols.

The compound can also be formulated for its topical application for the treatment of pathologies occurring in zones or organs accessible through this route, such as eyes, skin and the intestinal tract. Formulations include creams, lotions, gels, powders, solutions and patches wherein the compound is dispersed or dissolved in suitable excipients.

The activity of the compounds of the present invention can be determined using the following test:

Inhibition of α₄β₁-Dependent Cell Adhesion

Inhibition of α₄β₁-dependent cell adhesion was assayed by evaluating the interaction between the peptide CS-1 (H-CLHGPEILDVPST-CONH₂) and Jurkat cells (T lymphocyte cell line expressing activated integrin α₄β₁ but not integrin α₄β₇) after preincubation of said cells with the compounds of formula I to be tested. The peptide CS-1 was synthesized by conventional solid phase chemistry and purified by HPLC. Its identity was determined by elemental analysis and mass spectroscopy.

1.—Preparation of CS-1-Coated Plates

96-black well plates (Costar 3925) were used. 200 μL of 2% bovine serum albumin solution (BSA, Sigma α₄₅₀₃) was added per well and the plate was incubated for 2 hours at 37° C. The solution was discarded and the plate was washed twice with 200 μL of phosphate buffered saline solution (PBS, Gibco 14190-094) per well. Then, 200 μL of 10 μg/mL N-succinimidyl 3-(2-pyridyldithio)propionate solution (SPDP, Sigma P-3415) was added per well, and the plate was incubated at 37° C. for 30 minutes. The remaining solution was discarded and the plate was twice washed with 200 μL per well of PBS. Next, 200 μL of 25 μg/mL CS-1 peptide solution (equivalent to 5 μg/well) was added per well and the plate was incubated at 37° C. for 2 hours and then overnight at 4° C.

2.—Jurkat Cell Line: Culture and Fluorescent Labelling

Jurkat cells were kept in culture at a density comprised between 2×10⁵ and 1.5×10⁶ cells/mL in 1640 RPMI medium (Gibco 21875-034) enriched with 10% fetal calf serum (FCS, Gibco 10270-106).

50×10⁶ cells were extracted, centrifuged (1200 rpm, 10 min, room temperature) and the medium was discarded. Cells were resuspended in 5 mL of RPMI, and 10 μL of a 1 mM solution of the fluorophore calceine.AM (Molecular Probes, C-3100) was added. The suspension was incubated for 30 min at 37° C. in the darkness, with occasional shaking. 40 mL of RPMI was added to stop the labelling and the suspension was centrifuged (1200 rpm, 10 min, room temperature). The medium was discarded, and the labelled cells were washed with 40 mL of RPMI in order to remove the unincorporated fluorescent probe. Finally, cells were resuspended in RPMI enriched with 10% FCS to obtain a 8×10⁶ cells/mL suspension.

3.—Adhesion Experiment

The plate previously prepared was twice washed with 200 μL per well of PBS and blocked with 200 μL of 1% BSA for at least 1 hour at room temperature.

The products to be tested were dissolved in dimethylsulfoxide at a concentration of 10 mM and dilutions thereof were prepared in RPMI medium enriched with 10% FCS. The products were preincubated with the Jurkat cells at 37° C. for 30 minutes, at a product concentration comprised between 1 nM and 10 μM, at a cell density of 4×10⁶ cells/mL and at a maximal dimethylsulfoxide concentration of 0.1%.

100 μL of the resulting cell-product supsension was added per well (cellular density 4×10⁵ cells/well). The product was omitted in the control wells and cells or CS-1 were omitted in the blanks. The plate was incubated for 60 min at room temperature in the-darkness, was twice washed with 200 μL per well of RPMI and then 100 μL of PBS was added. The plate was read with a fluorescence reader at an excitation wavelength of 485 nm and at an emission wavelength of 530 nm.

Maximal adhesion (100%) was determined as the average of the fluorescence intensities (FI) of the control wells, and the minimal adhesion as the average FI of the blanks. The adhesion inhibition percentage was determined using the following formula:

$$\text{Inhibition} = 100 - \frac{{FI}_{product} - {FI}_{blank}}{{FI}_{control} - {FI}_{blank}} \times 100$$

The results are expressed as the IC₅₀ values (concentration producing 50% inhibition). IC₅₀ values were calculated by testing at least 6 product concentrations and by adjusting the inhibition percentages to a dose-response curve of variable slope.

The results obtained with representative compounds of the present invention, are shown in the following table.

| Compound | |
|---|---|
| (example n^{o}) | IC₅₀ (nM) |
| 54 | 22 |
| 55 | 58 |
| 56 | 23 |
| 58 | 166 |
| 59 | 13.3 |
| 60 | 11.6 |
| 61 | 2.3 |
| 62 | 188 |
| 63 | 198 |
| 64 | 193 |
| 65 | 45 |
| 66 | 3.5 |
| 67 | 33.0 |
| 70 | 17 |
| 71 | 37 |
| 72 | 15 |
| 73 | 57 |
| 74 | 48 |
| 75 | 101 |
| 76 | 67 |
| 77 | 247 |
| 79 | 7.6 |
| 80 | 0.5 |
| 81 | 33 |
| 82 | 89 |
| 83 | 65 |
| 84 | 6.0 |
| 85 | 12.0 |
| 86 | 18 |
| 87 | 14.1 |
| 88 | 83.9 |
| 89 | 7.7 |
| 91 | 64.6 |
| 93 | 26.7 |
| 94 | 14.1 |
| 95 | 4.9 |
| 96 | 74.8 |
| 97 | 122 |
| 98 | 17.7 |
| 99 | 13.1 |
| 102 | 305 |
| 105 | 120 |
| 116 | 9.8 |
| 117 | 135.4 |
| 119 | 49.8 |
| 123 | 4.6 |

The following examples illustrate, but do not limit, the scope of the present invention. The following abbreviations have been used in the examples:
EtOAc: ethyl acetate
DCC: dicyclohexylcarbodiimide
DEAD: diethyl azodicarboxylate
DIEA: diisopropylethylamine
DMF: dimethylformamide
EDC: N-[3-(dimethylamino)propyl]-N′-ethylcarbodiimide
HOBT: 1-hydroxybenzotriazole
HBTU: O-(benzotriazol-1-yl)-N,N,N′,N′-tetramethyluronium hexafluorophosphate
MeOH: methanol
NHS: N-hydroxysuccinimide
NMM: N-methylmorpholine
NMP: 1-methyl-2-pyrrolidinone
TEA: triethylamine
THF: tetrahydrofuran

### REFERENCE EXAMPLE 1

### 4-Aminomethyl-1-tert-butoxycarbonylpiperidine

To a solution of 4-(aminomethyl)piperidine (100 g, 0.88 mol) in CHCl₃ (550 mL), cooled to 0° C. and under argon atmosphere, di-tert-butyl dicarbonate (98 g 0.45 mol) dissolved in CHCl₃ (350 mL) was added. The resulting mixture was stirred at room temperature for 48 h. Next, it was washed with H₂O and the aqueous phase was reextracted with CHCl₃. The combined organic phases were dried over sodium sulfate and the solvents were removed to afford 84.5 g of the title compound (88% yield).

¹H NMR (80 MHz, CDCl₃) δ (TMS): 4.11 (broad d, J=13.4 Hz, 2H), 2.69 (m, 4H), 1.45 (s, 9H), 1.8-0.8 (complex signal, 7H).

### REFERENCE EXAMPLE 2

### (1-tert-Butoxycarbonylpiperidin-4-yl)methyl Mesylate

### a) 4-Piperidylmethanol

To a mixture of LiAlH₄ (8.82 g, 0.232 mol) and THF (125 mL), cooled to 0° C., a solution of ethyl isonipecotate (18 mL, 0.117 mol) in THF (325 mL) was added dropwise and under argon atmosphere, and the mixture was stirred overnight at room temperature. A mixture of H₂O (12.03 mL) and THF (25 mL), followed by a mixture of 15% NaOH (10.03 mL) and H₂O (32.4 mL) was slowly added at 0° C. The resulting mixture was filtered while washing with THF and the solvent was evaporated. The residue was partitioned between H₂O and CHCl₃, the phases were separated, the aqueous phase was extracted with CHCl₃ and the combined organic extracts were dried over Na₂SO₄ and concentrated. 8.2 g of the desired product was obtained (61% yield).

### b) (1-tert-Butoxycarbonylpiperidin-4-yl)methanol

To a solution of 4-piperidylmethanol (obtained in the preceding section) (15.3 g, 133 mmol) in DMF (160 mL) cooled to 0° C. and under argon atmosphere, di-tert-butyl dicarbonate (29 g, 133 mmol) dissolved in DMF (80 mL) was added, and the solution was stirred overnight at room temperature. It was concentrated to dryness and the residue was dissolved in a mixture of THF (100 mL), MeOH (100 mL), and NaOH 1N (100 mL) and stirred at room temperature for 18 h. The organic phase was evaporated and the aqueous phase was extracted three times with CHCl₃. The combined organic phases were dried over sodium sulfate and concentrated to dryness. 23.0 g of the desired product was obtained (80% yield).

### c) Title Compound

To a solution of the product obtained in the preceding section (6.8 g, 31 mmol) and DIEA (5.75 mL, 33 mmol) in CH₂Cl₂ (50 mL), cooled to 0° C. and under argon atmosphere, mesyl chloride (2.4 mL, 31 mmol) was added dropwise. The reaction mixture was stirred overnight at room temperature. Then, it was treated with H₂O, the phases were separated and the aqueous phase was reextracted with CH₂Cl₂. The combined organic phases were dried over sodium sulfate and concentrated, to afford the title compound in quantitative yield.

¹H NMR (300 MHz, CDCl₃) δ (TMS): 4.12 (broad d, J=11.8 Hz, 2H), 4.04 (d, J=6.5 Hz, 2H), 2.98 (s, 3H), 2.69 (broad t, J=12.4 Hz, 2H), 1.89 (m, 1H), 1.72 (broad d, J=12.9 Hz, 2H), 1.43 (s, 9H), 1.25 (m, 2H).

### REFERENCE EXAMPLE 3

### (4-Piperidylmethyl)pyrrolidin-2-one Hydrochloride

### a) 1-tert-Butoxycarbonyl-4-(3-chloropropylcarbonylaminomethyl)piperidine

To a solution of 4-aminomethyl-1-tert-butoxycarbonylpiperidine (obtained in reference example 1) (7 g, 33 mmol) and TEA (5.46 mL, 39 mmol) in CHCl₃ (250 mL), cooled to 0° C., a solution of 4-chlorobutyryl chloride (3.66 mL, 33 mmol) in CHCl₃ (10 mL) was added dropwise and under argon atmosphere. The reaction mixture was stirred overnight at room temperature and concentrated. The residue was treated with 0.2 M NaHCO₃ and CHCl₃. The phases were separated, the aqueous phase was reextracted with CHCl₃ and the combined organic phases were dried over sodium sulfate and concentrated. The desired compound was obtained in quantitative yield.

### b) 1-[(1-tert-Butoxycarbonylpiperidin-4-yl)methyl]pyrrolidin-2-one

To a solution of the compound obtained in the preceding section (10.5 g, 33 mmol) in DMF (160 mL), cooled to 0° C. and under argon atmosphere, potassium tert-butoxide (3.8 g, 34 mmol) was slowly added, and the mixture was stirred overnight at room temperature. The reaction mixture was concentrated to dryness and treated with phosphate buffer and CHCl₃. The phases were separated, the aqueous phase was reextracted with CHCl₃ and the combined organic phases were dried over sodium sulfate and concentrated. A crude product was obtained, which was purified by chromatography on silica gel, using CH₂Cl₂/MeOH mixtures of increasing polarity as eluent. 4.3 g of the desired compound was obtained (33% yield).

### c) Title Compound

The compound obtained in the preceding section (4.3 g, 11 mmol) and a 4 M dioxane/HCl_{(g)} mixture (30 mL) were mixed in a flask under argon atmosphere. The mixture was stirred overnight at room temperature and concentrated to dryness by adding CH₂Cl₂ at the end of the evaporation process. 2.4 g of the title compound was obtained (quantitative yield).

¹H NMR (300 MHz, CD₃OD+CDCl₃) δ (TMS): 4.50 (s, 2H), 3.40 (m, 2H), 3.30 (m, 2H), 3.17 (m, 2H), 2.85 (m, 2H), 2.42 (m, 2H), 2.05 (m, 2H), 1.85 (m, 1H), 1.90 (m, 2H), 1.49 (m, 2H).

### REFERENCE EXAMPLE 4

### 2-(4-Piperidylmethyl)isoindolin-1-one Hydrochloride

### a) 2-[(1-tert-Butoxycarbonylpiperidin-4-yl)methyl]isoindolin-1-one

To a solution of 4-aminomethyl-1-tert-butoxycarbonylpiperidine (obtained in reference example 1) (6 g, 28 mmol) and 2-formylbenzoic acid (4.8 g, 32 mmol) in MeOH (53 mL) and H₂O (5.3 mL), sodium cyanoborohydride (2.64 g, 42 mmol) was slowly added, and the reaction mixture was stirred at reflux overnight. 10% NaOH (6.3 mL) was added and MeOH was evaporated. The mixture was extracted with EtOAc and the combined organic phases were dried over sodium sulfate and concentrated. The resulting crude product was purified by chromatography on silica gel, using EtOAc as eluent. 2.15 g of the desired compound was obtained (23% yield).

### b) Title Compound

Following a similar procedure to that described in section c of reference example 3, but using 2-[(1-tert-butoxycarbonylpiperidin-4-yl)methyl]isoindolin-1-one (obtained in the preceding section) as starting product, the title compound of the example was obtained in quantitative yield.

¹H NMR (300 MHz, CD₃OD) δ (TMS): 7.76 (m, 1H), 7.58 (m, 2H), 7.49 (m, 1H), 4.84 (s, 2H), 4.55 (s, 2H), 3.58 (d, J=7.2 Hz, 2H), 3.40 (d, J=12.7 Hz, 2H), 2.98 (broad t, J=12.3 Hz, 2H), 2.16 (m, 1H), 1.92 (broad d, J=14.2 Hz, 2H), 1.48 (m, 2H).

### REFERENCE EXAMPLE 5

### 1-(4-Piperidylmethyl)-2,5-pyrrolidin-2,5-dione Hydrochloride

### a) 1-[(tert-Butoxycarbonylpiperidin-4-yl)methyl]pyrrolidin-2,5-dione

To a solution of 4-aminomethyl-1-tert-butoxycarbonylpiperidine (obtained in reference example 1) (5 g, 23 mmol) in CH₂Cl₂ (50 mL), succinic anhydride (4.7 g, 46 mmol) was slowly added under argon atmosphere, and the resulting mixture was stirred at room temperature for 3 h. Next, the reaction mixture was cooled to 0° C., 1,1′-carbonyldiimidazole was slowly added and the mixture was stirred overnight at room temperature. The solvent was evaporated and the residue obtained was treated with H₂O and EtOAc. It was filtered, the phases were separated and the organic phase was washed with 40% NaHSO₄ solution, dried over sodium sulfate and concentrated, to afford 5.9 g of the desired compound (87% yield).

### b) Title Compound

Following a similar procedure to that described in section c of reference example 3, but using 1-[(tert-butoxycarbonylpiperidin-4-yl)methyl]pyrrolidin-2,5-dione (obtained in the preceding section) as starting product, the title compound of the example was obtained in 76% yield.

¹H NMR (300 MHz, CD₃OD+CDCl₃) δ (TMS): 3.88 (s, 2H), 3.41 (d, J=6.8 Hz, 2H), 3.33 (broad d, J=12.8 Hz, 2H), 2.82 (m, 2H), 2.71 (s, 4H), 1.90 (m, 3H), 1.51 (m, 2H).

### REFERENCE EXAMPLE 6

### 3-[(4-Piperidyl)methyl]oxazolidin-2-one Hydrochloride

### a) 1-tert-Butoxycarbonyl-4-(2-chloroethoxycarbonylaminomethyl)piperidine

Following a similar procedure to that described in section a of reference example 3, but using 2-chloroethyl chloroformate instead of 4-chlorobutyryl chloride, the desired compound was obtained in 78% yield.

### b) 3-[(1-tert-Butoxycarbonylpiperidin-4-yl)methyl]oxazolidin-2-one

To a solution of 1-tert-butoxycarbonyl-4-(2-chloroethoxycarbonylaminomethyl)piperidine (obtained in the preceding section) (4 g, 12 mmol) in DMF (60 mL), 50% sodium hydride (0.6 g, 12 mmol) was slowly added under argon atmosphere, and the reaction mixture was stirred overnight at 40° C. Some drops of H₂O were added and the mixture was evaporated to dryness. The residue was partitioned between phosphate buffer (pH=7.8) and CH₂Cl₂, the phases were separated, the aqueous phase was reextracted with CH₂Cl₂ and the combined organic phases were dried over sodium sulfate and concentrated. The resulting crude product was purified by chromatography on silica gel, using hexane/EtOAc mixtures of increasing polarity as eluent. 2.1 g of the desired compound was obtained (59% yield).

### c) Title Compound

Following a similar procedure to that described in section c of reference example 3, but using 3-[(1-tert-butoxycarbonylpiperidin-4-yl)methyl]oxazolidin-2-one (obtained in the preceding section) as starting product, the title compound of the example was obtained in quantitative yield.

¹H NMR (300 MHz, CD₃OD) δ (TMS): 4.34 (m, 2H), 4.77 (s, 2H), 3.92 (m, 2H), 3.44 (m, 2H), 3.17 (m, 1H), 3.02 (m, 3H), 2.04 (m, 2H), 1.50 (m, 2H).

### REFERENCE EXAMPLE 7

### 2-Phenyl-1-[(4-piperidyl)methyl]imidazole

### a) 1-[(1-tert-Butoxycarbonylpiperidin-4-yl)methyl]-2-phenylimidazole

Following a similar procedure to that described in section b of reference example 6, but using THF as solvent and (1-tert-butoxycarbonylpiperidin-4-yl)methyl mesylate (obtained in reference example 2) and 2-phenylimidazole as reagents instead of 1-tert-butoxycarbonyl-4-(2-chloroethoxycarbonylaminomethyl)piperidine, and carrying out the reaction at reflux, the desired compound was obtained in 61% yield.

### b) Title Compound

The compound obtained in the preceding section (1.1 g, 3 mmol) and a 4 M dioxane/HCl_{(g)} mixture (10 mL) were mixed in a flask under argon atmosphere. The mixture was stirred overnight at room temperature and concentrated to dryness. The residue obtained was partitioned between 1 N NaOH solution and CHCl₃. The phases were separated and the organic phase was dried over sodium sulfate and concentrated to dryness, to afford 0.62 g of the title compound of the example (87% yield).

¹H NMR (300 MHz, CDCl₃) δ (TMS): 7.40-7.90 (complex signal, 5H), 7.10 (d, J=1 Hz, 1H), 6.96 (d, J=1 Hz, 1H), 3.87 (d, J=7.4 Hz, 2H), 3.57 (m, 1H), 3.01 (m, 2H), 2.51 (m, 2H), 1.74 (m, 1H), 1.46 (m, 2H), 1.11 (m, 2H).

### REFERENCE EXAMPLE 8

### 2-Methyl-1-(4-piperidylmethyl)imidazo[4,5-c]pyridine

### a) 4-[[(1-tert-Butoxycarbonylpiperidin-4-yl)methyl]amino]-3-nitropyridine

Following a similar procedure to that described in section a of reference example 3, but using 4-chloro-3-nitropyridine instead of 4-chlorobutyryl chloride, the desired compound was obtained in 48% yield.

### b) 3-Amino-4-[[(1-tert-butoxycarbonylpiperidin-4-yl)methyl]amino]pyridine

A solution of the product obtained in the preceding section (26.2 g, 77 mmol) in MeOH (500 mL) was hydrogenated under atmospheric pressure in the presence of 10% Pd/C (3.83 g) for 18 h. The catalyst was filtered off and the solvent removed, to afford 22.9 g of a crude product that was directly used in the following step.

### c) 1-[(1-tert-Butoxycarbonylpiperidin-4-yl)methyl]-2-methylimidazo[4,5-c]pyridine

To a solution of 3-amino-4-[[(1-tert-butoxycarbonylpiperidin-4-yl)methyl]amino]pyridine (obtained in the preceding section) (22.9 g, 70 mmol) in EtOH (350 mL), ethyl acetimidate hydrochloride (9.2 g, 74 mmol) was added under argon atmosphere, and the mixture was heated at reflux for 4 h. Next, a second equivalent of ethyl acetimidate hydrochloride (9.2 g, 74 mmol) was added and the mixture was further heated at reflux for 18 h and a third equivalent of ethyl acetimidate hydrochloride (9.2 g, 74 mmol) was added and the mixture was further heated for 4 h. The resulting solution was concentrated in vacuo and was partitioned between CHCl₃ and 0.5 N NaOH solution. The organic phase was dried over sodium sulfate and the solvents were removed to afford 30 g of a crude product, which was purified by chromatography on silica gel (CHCl₃:MeOH 10%), to yield 23.4 g of a yellow solid (95% yield);

### d) Title Compound

Following a similar procedure to that described in section b of reference example 7, but using 1-[(1-tert-butoxycarbonylpiperidin-4-yl)methyl]-2-methylimidazo[4,5-c]pyridine (obtained in the preceding section) as starting product, 15.8 g of the title compound of the example was obtained (98% yield).

¹H NMR (80 MHz, CDCl₃) δ (TMS): 8.96 (s, 1H), 8.35 (d, J=5.5 Hz, 1H), 7.20 (d, J=5.5 Hz, 1H), 3.95 (d, J=7.3 Hz, 2 H), 3.06 (broad d, J=12.0 Hz, 2H), 2.61 (s, 3H), 2.51 (broad t, J=12.7 Hz, 2H), 2.2-1.0 (complex signal, 6H).

### REFERENCE EXAMPLE 9

### 2-Ethyl-5,7-dimethyl-3-(4-piperidylmethyl)imidazo[4,5-b]pyridine Dihydrochloride

### a) 3-[(1-tert-Butoxycarbonylpiperidin-4-yl)methyl]-2-ethyl-5,7-dimethylimidazo[4,5-b]pyridine

Following a similar procedure to that described in section b of reference example 6, but using (1-tert-butoxycarbonylpiperidin-4-yl)methyl mesylate (obtained in reference example 2) and 2-ethyl-5,7-dimethyl-3H-imidazo[4,5-b]pyridine (obtained as described in EP 400974) as reagents, instead of 1-tert-butoxycarbonyl-4-(2-chloroethoxycarbonylaminomethyl)piperidine, and carrying out the reaction at room temperature the desired compound was obtained in 61% yield.

### b) Title Compound

Following a similar procedure to that described in section c of reference example 3, but using 3-[(1-tert-butoxycarbonylpiperidin-4-yl)methyl]-2-ethyl-5,7-dimethylimidazo[4,5-b]pyridine (obtained in the preceding section) as starting product, the title compound of the example was obtained in quantitative yield.

¹H NMR (300 MHz, CD₃OD) δ (TMS): 7.36 (s, 1H), 4.47 (d, J=7.4 Hz, 2H), 4.78 (s, 3H), 3.67 (m, 2H), 3.36 (m, 2H), 2.98 (m, 2H), 2.66 (s, 3H), 2.65 (s, 3H), 2.44 (m, 1H), 1.92 (m, 2H), 1.71 (m, 2H), 1.56 (t, J=7.5 Hz, 3H).

### REFERENCE EXAMPLE 10

### 1-(2-Ethoxyethyl)-2-[(1-piperazinyl)methyl]benzimidazole

### a) 2-[(4-Formylpiperazin-1-yl)methyl]benzimidazole

To a solution of 1-formylpiperazine (23.1 mL, 0.225 mol) in ethanol (70 mL), heated at 60° C. under argon atmosphere, a solution of 2-chloromethylbenzimidazole (15 g, 0.090 mol) in ethanol (100 mL) and DMF (25 mL) was added. The mixture was stirred at 60° C. for 2 h, the solvent was evaporated and the resulting residue was treated with 4 N NaOH solution (40 mL). It was extracted with CHCl₃, the combined organic phases were dried over sodium sulfate and concentrated to dryness, to give 30 g of a crude product, which was directly used in the following step.

### b) 1-(2-Ethoxyethyl)-2-[(4-formylpiperazin-1-yl)methyl]benzimidazole

Following a similar procedure to that described in section b of reference example 6, but using the crude product obtained in the preceding section and 2-bromoethyl ethyl ether as reagents, instead of 1-tert-butoxycarbonyl-4-(2-chloroethoxycarbonylaminomethyl)piperidine, and carrying out the reaction at 60° C., the desired compound was obtained in 32% yield.

### c) Title Compound

A solution of 1-(2-ethoxyethyl)-2-[(4-formylpiperazin-1-yl)methyl]benzimidazole (obtained in the preceding section) (2 g, 6 mmol) in MeOH (30 mL) and 10% HCl (17 mL) was heated at reflux for 1 hour. MeOH was evaporated and the residue was treated with 1 N NaOH solution and was extracted with CHCl₃. The organic phase was dried over sodium sulfate and concentrated to dryness to afford a crude product which was purified by chromatography on silica gel using CHCl₃/MeOH/NH₃ mixtures of increasing polarity as eluent. 0.94 g of the title compound of the example was obtained (50% yield).

¹H NMR (300 MHz, CDCl₃) δ (TMS): 7.70 (m, 1H), 7.37 (m, 1H), 7.25 (m, 2H), 4.52 (m, 2H), 3.84 (s, 2H), 3.74 (m, 2H), 3.40 (q, J=7 Hz, 2H), 2.85 (m, 4H), 2.48 (m, 4H), 1.69 (broad s, 1H), 1.12 (t, J=7 Hz, 3H).

### REFERENCE EXAMPLE 11

### 1-(2-Pyridylmethyl)piperazine

### a) 2-Chloromethylpyridine Hydrochloride

To a solution of 2-pyridylmethanol (141.88 mL, 1.47 mol) in anhydrous CH₂Cl₂ (800 mL) cooled to 0° C. and under argon atmosphere, SOCl₂ (104.4 mL, 1.43 mol) was added dropwise. The reaction mixture was heated at reflux for 2 hours, allowed to cool to room temperature and was concentrated to dryness. The crude product obtained was directly used in the following step.

### b) Title Compound

To a solution of piperazine (124 g, 1.44 mol) in H₂O (500 mL) cooled to 0° C., was added first 37% HCl (129 mL, 1.57 mol) and then a solution of 2-chloromethylpyridine hydrochloride (obtained in the preceding section) (0.735 mols) in H₂O (400 mL) dropwise. The resulting mixture was stirred at room temperature for 48 hours and was extracted with EtOAc. The resulting aqueous phase was extracted several times with the same solvent at different pH: progressively basifying by addition of solid NaOH, it was firstly extracted at pH 7, then at a slightly basic pH and finally at basic pH. The organic phase resulting from this last extraction was dried over sodium sulfate and concentrated to dryness to afford 204 g of the title compound of the example (80% yield).

¹H NMR (300 MHz, CD₃OD) δ (TMS): 8.47 (m, 1H), 7.80 (m, 1H), 7.53 (m, 1H), 7.32 (m, 1H), 4.85 (broad s, 1H), 3.67 (s, 2H), 3.00 (m, 4H), 2.58 (m, 4H).

### REFERENCE EXAMPLE 12

### 1-(2-Thienylmethyl)piperazine

A suspension of 2-thienylmethanamine (5.0 g, 44 mmol), bis-(2-chloroethyl)amine hydrochloride (7.8 g, 44 mmol) and K₂CO₃ (3.1 g, 22 mmol) in 1-butanol (30 mL) was heated at reflux overnight and under argon atmosphere. A second equivalent of K₂CO₃ (3.1 g, 22 mmol) was added and the suspension was further heated at reflux overnight. The resulting mixture was concentrated to dryness and the residue was partitioned between CHCl₃ and 0.5 N NaOH solution. The phases were separated, the organic phase was dried over sodium sulfate and the solvent was evaporated. The resulting crude product was purified by chromatography on silica gel using CHCl₃/MeOH/NH₃ mixtures of increasing polarity as eluent. 0.28 g of the title compound of the example was obtained (3% yield).

¹H NMR (300 MHz, CD₃OD) δ (TMS): 7.28 (m, 1H), 6.96 (m, 2H), 4.83 (s, 1H), 3.73 (s, 2H), 2.83 (m, 4H), 2.41 (m, 4H).

### REFERENCE EXAMPLE 13

### 1-(2-Aminoethyl)-4-tert-butoxycarbonylpiperazine

### a) 1-(2-Benzylidenaminoethyl)piperazine

A mixture of 1-(2-aminoethyl)piperazine (10.2 mL, 78.9 mmol), benzaldehyde (8.1 mL, 78.9 mmol) and toluene (100 mL) under argon atmosphere was heated at reflux in a Dean-Stark overnight. The resulting mixture was allowed to cool to room temperature and was directly used in the following step.

### b) Title Compound

To the mixture obtained in the preceding section, cooled to 0° C., di-tert-butyl dicarbonate (17.2 g, 78.9 mmol) was slowly added, and the resulting mixture was stirred overnight at room temperature. It was concentrated to dryness and the residue was treated with 1 N KHSO₄ solution. It was washed three times with EtOAc and basified while cooling with 2 N NaOH solution. CHCl₃ was added, the phases were separated, and the combined organic extracts were dried over anhydrous Na₂SO₄ and concentrated to dryness. The title compound was obtained in 77% yield.

¹H NMR (300 MHz, CDCl₃) δ (TMS); 3.43 (m, 4H), 2.78 (m, 2H), 2.39 (m, 6H), 1.56 (broad s, 2H), 1.44 (s, 9H).

### REFERENCE EXAMPLE 14

### 4-[(3-Methylbutanoylamino)methyl]piperidine Hydrochloride

### a) 1-tert-Butoxycarbonyl-4-[(3-methylbutanoylamino)methyl]piperidine

Following a similar procedure to that described in section a of reference example 3, but using isopentanoyl chloride instead of 4-chlorobutanoyl chloride, the desired compound was obtained in 46% yield.

### b) Title Compound

Following a similar procedure to that described in section c of reference example 3, but using 1-tert-butoxycarbonyl-4-[(isobutylcarbonylamino)methyl]piperidine (obtained in the preceding section) as starting product, the title compound of the example was obtained in quantitative yield.

¹H NMR (300 MHz, CD₃OD) δ (TMS): 4.88 (s, 3H), 3.33 (m, 2H), 3.14 (d, J=5.9 Hz, 2H), 2.96 (broad t, J=12.2 Hz, 2H), 2.08 (m, 2H), 2.05 (m, 1H), 1.90 (m, 3H), 1.40 (m, 2H), 0.94 (d, J=6.1 Hz, 6H).

### REFERENCE EXAMPLE 15

### N-tert-Butyl-N′-(4-piperidylmethyl)urea Hydrochloride

### a) N-tert-Butyl-N′-[(1-tert-butoxycarbonylpiperidin-4-yl)methyl]urea

To a solution of 4-aminomethyl-1-tert-butoxycarbonylpiperidine (obtained in reference example 1) (5 g, 23 mmol) in DMF (20 mL), tert-butyl isocyanate (2.63 mL, 23 mmol) was added dropwise and under argon atmosphere. The reaction mixture was stirred overnight at room temperature and was concentrated to dryness to afford the desired compound in quantitative yield.

### b) Title Compound

Following a similar procedure to that described in section c of reference example 3, but using N-tert-butyl-N′-[(1-tert-butoxycarbonylpiperidin-4-yl)methyl]urea (obtained in the preceding section) as starting product, the title compound of the example was obtained in quantitative yield.

¹H NMR (300 MHz, CD₃OD) δ (TMS): 4.92 (s, 4H), 3.37 (m, 2H), 2.97 (m, 4H), 1.95 (m, 2H), 1.80 (m, 1H), 1.43 (m, 2H), 1.31 (s, 9H).

### REFERENCE EXAMPLE 16

### Isobutyl (4-piperidylmethyl)carbamate Hydrochloride

### a) isobutyl [(1-tert-butoxycarbonylpiperidin-4-yl)methyl]carbamate

Following a similar procedure to that described in section a of reference example 3, but using isobutyl chloroformate instead of 4-chlorobutyryl chloride, the desired compound was obtained in 69% yield.

### b) Title Compound

Following a similar procedure to that described in section c of reference example 3, but using isobutyl [(1-tert-butoxycarbonylpiperidin-4-yl)methyl]carbamate (obtained in the preceding section) as starting product, the title compound of the example was obtained in quantitative yield.

¹H NMR (300 MHz, CDCl₃) δ (TMS): 9.67 (broad s, 1H), 9.65 (broad s, 1H), 4.84 (m, 1H), 3.84 (m, 2H), 3.51 (broad d, J=12.1 Hz, 2H), 3.27 (m, 1H), 3.10 (m, 2H), 2.83 (m, 2H), 1.97 (m, 2H), 1.84 (m, 1H), 1.62 (m, 2H), 0.91 (d, J=6.8 Hz, 6H).

### REFERENCE EXAMPLE 17

### N-(4-Piperidylmethyl)isopropanesulfonamide Hydrochloride

### a) N-[(1-tert-Butoxycarbonylpiperidin-4-yl)methyl]isopropanesulfonamide

Following a similar procedure to that described in section a of reference example 3, but using isopropylsulfonyl chloride instead of 4-chlorobutyryl chloride, the desired compound was obtained in 9% yield.

### b) Title Compound

Following a similar procedure to that described in section c of reference example 3, but using N-[(1-tert-butoxycarbonylpiperidin-4-yl)methyl]isopropanesulfonamide (obtained in the preceding section) as starting product, the title compound of the example was obtained in quantitative yield.

¹H NMR (300 MHz, CD₃OD) δ (TMS): 5.04 (s, 3H), 3.52 (m, 2H), 3.17 (m, 5H), 2.18 (m, 2H), 2.01 (m, 1H), 1.60 (m, 2H), 1.49 (d, J=7.7 Hz, 6H).

### REFERENCE EXAMPLE 18

### N-Isopropyl-N′-(4-piperidylmethyl)thiourea Hydrochloride

### a) N-[(1-tert-Butoxycarbonylpiperidin-4-yl)methyl]-N′-isopropylthiourea

To a solution of 4-aminomethyl-1-tert-butoxycarbonylpiperidine (obtained in reference example 1) (3 g, 14 mmol) in THF (20 mL), cooled to 0° C. and under argon atmosphere, isopropyl isothiocyanate (1.5 mL, 14 mmol) was added dropwise. The reaction mixture was stirred overnight at room temperature and was treated with 0.2 M NaHCO₃. The phases were separated, the aqueous phase was reextracted with CHCl₃ and the combined organic phases were dried over sodium sulfate and concentrated. The desired compound was obtained in 48% yield.

### b) Title Compound

Following a similar procedure to that described in section c of reference example 3, but using N-[(1-tert-butoxycarbonylpipeidin-4-yl)methyl]-N′-isopropylthiourea (obtained in the preceding section) as starting product, the title compound of the example was obtained in quantitative yield.

¹H NMR (300 MHz, CD₃OD) δ (TMS): 4.84 (s, 4H), 4.30 (m, 1H), 3.45 (m, 4H), 2.96 (broad t, J=12.7 Hz, 2H), 1.95 (m, 3H), 1.43 (m, 2H), 1.17 (d, J=6.5 Hz, 6H).

### REFERENCE EXAMPLE 19

### 4-(1-Pyrrolylmethyl)piperidine

### a) 1-tert-Butoxycarbonyl-4-(1-pyrrolylmethyl)piperidine

To a solution of 4-aminomethyl-1-tert-butoxycarbonylpiperidine (obtained in reference example 1) (5 g, 23.3 mol) in acetic acid (123.7 mL), 2,5-dimethoxytetrahydrofuran (3.0 mL, 23.3 mol) was slowly added under argon atmosphere. The reaction mixture was heated at reflux for 1 h, concentrated to dryness and the resulting crude product was purified by chromatography on silica gel using EtOAc/hexane mixtures of increasing polarity as eluent. The desired compound was obtained in 37% yield.

### b) Title Compound

Following a similar procedure to that described in section b of reference example 7, but using 1-tert-butoxycarbonyl-4-(1-pyrrolylmethyl)piperidine (obtained in the preceding section) as starting product and purifying the crude product obtained by chromatography on silica gel using CHCl₃/MeOH mixtures of increasing polarity as eluent, the title compound of the example was obtained in 14% yield.

¹H NMR (300 MHz, CDCl₃) δ (TMS): 6.62 (m, 2H), 6.13 (m, 2H), 3.72 (d, J=7.2 Hz, 2H), 3.05 (m, 2H), 2.55 (m, 2H), 1.79 (m, 1H), 1.60 (m, 3H), 1.20 (m, 2H).

### REFERENCE EXAMPLE 20

### 4-[(2,5-Dimethylpyrrol-1-yl)methyl]piperidine Hydrochloride

### a) 1-tert-Butoxycarbonyl-4-[(2,5-dimethylpyrrol-1-yl)methyl]piperidine

Following a similar procedure to that described in section a of reference example 19, but using acetonylacetone instead of 2,5-dimethoxytetrahydrofuran, the desired compound was obtained in 34% yield.

### b) Title Compound

Following a similar procedure to that described in section c of reference example 3, but using 1-tert-butoxycarbonyl-4-[(2,5-dimethylpyrrol-1-yl)methyl]piperidine (obtained in the preceding section) as starting product, the title compound of the example was obtained in quantitative yield.

¹H NMR (300 MHz, CD₃OD) δ (TMS): 5.58 (d, J=3.5 Hz, 2H), 4.85 (s, 2H), 3.72 (d, J=7.5 Hz, 2H), 3.39 (m, 2H), 2.93 (m, 2H), 2.19 (s, 6H), 2.03 (m, 1H), 1.81 (m, 2H), 1.20 (m, 2H).

### REFERENCE EXAMPLE 21

### 4-(Dimethylaminomethyl)piperidine Dihydrochloride

### a) 1-tert-Butoxycarbonyl-4-(dimethylaminomethyl)piperidine

To a solution of 4-aminomethyl-1-tert-butoxycarbonylpiperidine (obtained in reference example 1) (5 g, 23.3 mmol) in acetonitrile, 37% aqueous formaldehyde (17.5 mL, 233.3 mmol) and sodium cyanoborohydride (4.4 g, 70.0 mmol) were added, and the reaction mixture was stirred at room temperature for 15 min. It was brought to pH 7 by the addition of glacial acetic acid (2.3 mL) and was stirred for 45 min. It was concentrated to dryness and the residue was treated with 2 N NaOH and was extracted with CHCl₃. The combined organic extracts were dried over anhydrous Na₂SO₄ and concentrated, to afford a crude product which was purified by chromatography on silica gel using CHCl₃/MeOH mixtures of increasing polarity as eluent. 851 mg of the desired product was obtained (9% yield).

### b) Title Compound

Following a similar procedure to that described in section c of reference example 3, but using 1-tert-butoxycarbonyl-4-(dimethylaminomethyl)piperidine (obtained in the preceding section) as starting product, the title compound of the example was obtained in quantitative yield.

¹H NMR (300 MHz, CD₃OD+CDCl₃) δ (TMS): 4.17 (s, 3H), 3.36 (m, 2H), 2.98 (m, 4H), 2.83 (s, 6H), 2.17 (m, 1H), 2.02 (m, 2H), 1.63 (m, 2H).

### REFERENCE EXAMPLE 22

### 1-[2-(Dimethylamino)ethyl]piperazine Hydrochloride

### a) 4-tert-Butoxycarbonyl-1-[2-(dimethylamino)ethyl]piperazine

Following a similar procedure to that described in section a of reference example 21 but starting from 1-(2-aminoethyl)₄-tert-butoxycarbonylpiperazine (obtained in reference example 13) instead of 4-aminomethyl-1-tert-butoxycarbonylpiperidine, the desired compound was obtained in 45% yield.

¹H NMR (300 MHz, CDCl₃) δ (TMS): 3.42 (m, 4H), 2.45 (m, 8H), 2.24 (s, 6H), 1.44 (s, 9H).

### b) Title Compound

Following a similar procedure to that described in section c of reference example 3, but using 4-tert-butoxycarbonyl-1-[2-(dimethylamino)ethyl]piperazine (obtained in the preceding section) as starting product, the title compound of the example was obtained in quantitative yield.

### REFERENCE EXAMPLE 23

### 2-Ethyl-1-(4-piperidylmethyl)imidazo[4,5-c]pyridine Dihydrochloride

### a) 1-[(1-tert-Butoxycarbonylpiperidin-4-yl)methyl]-2-ethylimidazo[4,5-c]pyridine

To a solution of 3-amino-4-[[(1-tert-butoxycarbonylpiperidin-4-yl)methyl]amino]pyridine (obtained in section b of reference example 8) (2 g, 5.94 mmol) in DMF (16 mL), catalytic para-toluenesulfonic acid was first added, and then triethyl orthopropionate (7.3 g, 41.5 mmol) was added dropwise. The reaction mixture was heated at 120° C. for 1 h, DMF was evaporated and the residue was partitioned between CHCl₃ and 0.1 M NaOH. The combined organic phases were washed with H₂O, dried over Na₂SO₄ and concentrated to dryness. The crude product obtained was purified by chromatography on silica gel using CHCl₃/MeOH (9:1) as eluent, to afford the desired compound in 7% yield.

### b) Title Compound

Following a similar procedure to that described in section c of reference example 3, but using 1-[(1-tert-butoxycarbonylpiperidin-4-yl)methyl]-2-ethylimidazo[4,5-c]pyridine (obtained in the preceding section) as starting product, the title compound of the example was obtained in quantitative yield.

¹H NMR (300 MHz, CD₃OD+CDCl₃) δ (TMS): 9.00 (S, 1H), 8.47 (m, 2H), 4.40 (s, 3H), 4.36 (m, 2H), 3.37 (m, 2H), 3.07 (q, J=7.4 Hz, 2H), 2.90 (m, 2H), 2.26 (m, 1H), 1.91 (m, 2H), 1.66 (m, 2H), 1.51 (t, J=7.4 Hz, 3H).

### REFERENCE EXAMPLE 24

### 3H-1-(4-Piperidylmethyl)imidazo[4,5-c]pyridin-2-one Dihydrochloride

### a) 3H-1-(1-tert-Butoxycarbonylpiperidin-4-yl)methyl]imidazo[4,5-c]pyridin-2-one

Following a similar procedure to that described in section a of reference example 5, but using only 3-amino-4-[[(1-tert-butoxycarbonylpiperidin-4-yl)methyl]amino]pyridine (obtained in section b of reference example 8) and 1,1′-carbonyldiimidazole as reagents, the desired compound was obtained in 6% yield.

### b) Title Compound

Following a similar procedure to that described in section c of reference example 3, but using 3H-1-[(1-tert-butoxycarbonylpiperidin-4-yl)methyl]imidazo[4,5-c]pyridin-2-one (obtained in the preceding section) as starting product, the title compound of the example was obtained in 68% yield.

¹H NMR (300 MHz, CD₃OD) δ (TMS): 8.46 (m, 2H), 7.83 (m, 1H), 4.82 (s, 4H), 3.99 (d, J=7.2 Hz, 2H), 3.41 (broad d, J=12.9 Hz, 2H), 2.97 (m, 2H), 2.26 (m, 1H), 1.95 (m, 2H), 1.60 (m, 2H).

### REFERENCE EXAMPLE 25

### 4-[2-(1-Pyrrolidinylcarbonyloxy)ethyl]piperidine Hydrochloride

### a) 2-(1-tert-Butoxycarbonylpiperidin-4-yl)ethanol

Following a similar procedure to that described in section b of reference example 2, but starting from 2-(4-piperidyl)ethanol instead of 4-piperidylmethanol, the desired compound was obtained in 88% yield.

### b) 1-tert-Butoxycarbonyl-4-[2-(phenoxycarbonyloxy)ethyl]piperidine

Following a similar procedure to that described in section a of reference example 3, but using the compound obtained in the preceding section instead of 4-aminomethyl-1-tert-butoxycarbonylpiperidine and using phenyl chloroformate instead of 4-chlorobutyryl chloride, the desired compound was obtained in 93% yield.

### c) 1-tert-Butoxycarbonyl-4-[2-(1-pyrrolidinylcarbonyloxy)ethyl]piperidine

To a solution of the compound obtained in the preceding section (1.5 g, 4.29 mmol) in pyridine (10 mL), pyrrolidine (0.36 mL, 4.29 mmol) was added under argon atmosphere, and the resulting mixture was heated at 80° C. overnight. It was concentrated to dryness and the residue obtained was partitioned between H₂O and CHCl₃. The phases were separated and the combined organic extracts were dried over anhydrous Na₂SO₄ and concentrated. A crude product was obtained, which was purified by chromatography on silica gel using EtOAc/hexane (1:1) as eluent. 0.49 g of the desired compound was obtained (35% yield).

### d) Title Compound

Following a similar procedure to that described in section c of reference example 3, but using the compound obtained in the preceding section as starting product, the title compound of the example was obtained in quantitative yield.

¹H NMR (300 MHz, CDCl₃) δ (TMS): 9.62 (broad s, 1H), 9.30 (broad s, 1H), 4.13 (t, J=5.2 Hz, 2H), 3.47 (m, 2H), 3.36 (m, 4H), 2.87 (m, 2H), 1.93 (m, 5H), 1.60 (m, 6H).

### REFERENCE EXAMPLE 26

### 2-[(4-Piperidylmethyl)amino]-4-(trifluoromethyl)pyrimidine Dihydrochloride

### a) 2-[[(1-tert-Butoxycarbonylpiperidin-4-yl)methyl]amino]4-(trifluoromethyl)pyrimidine

Following a similar procedure to that described in section a of reference example 3, but using 2-chloro-4-(trifluoromethyl)pyrimidine instead of 4-chlorobutyryl chloride and using acetonitrile as solvent, the desired compound was obtained in 82% yield.

### b) Title Compound

Following a similar procedure to that described in section c of reference example 3, but using 2-[[(1-tert-butoxycarbonylpiperidin-4-yl)methyl]amino]4-(trifluoromethyl)pyrimidine (obtained in the preceding section) as starting product, the title compound of the example was obtained in quantitative yield.

¹H NMR (300 MHz, CD₃OD) δ (TMS): 8.51 (d, J=4.9 Hz, 1H), 6.92 (d, J=8.9 Hz, 1H), 4.83 (s, 4H), 3.65 (m, 1H), 3.56 (m, 1H), 3.39 (m, 2H), 2.97 (m, 2H), 2.01 (m, 3H), 1.48 (m, 2H).

### REFERENCE EXAMPLE 27

### 1-Methyl [(4-piperidyl)methyl]piperazine Trihydrochloride

### a) 4-[(1-tert-Butoxycarbonylpiperidin-4-yl)methyl]-1-methylpiperazine

To a solution of (1-tert-butoxycarbonylpiperidin-4-yl)methyl mesylate (obtained in reference example 2, 1.0 g, 3.48 mmol) in NMP, 1-methylpiperazine (0.75 mL, 6.8 mmol) was added under argon atmosphere, and the resulting mixture was heated at 85° C. for 48 h. It was concentrated to dryness and the residue obtained was partitioned between 0.2 M NaHCO₃ and CHCl₃. The phases were separated and the organic phase was dried over anhydrous Na₂SO₄ and concentrated. The crude product obtained was purified by chromatography on silica gel using CHCl₃/MeOH 2% as eluent. 0.61 g of the desired compound was obtained (62% yield).

### b) Compound Tituar

Following a similar procedure to that described in section c of reference example 3, but using 4-[(1-tert-butoxycarbonylpiperidin-4-yl)methyl]-1-methylpiperazine (obtained in the preceding section) as starting product, the title compound of the example was obtained in quantitative yield.

¹H NMR (300 MHz, CD₃OD) δ (TMS): 4.84 (s, 4H), 3.66 (m, 2H), 3.42 (m, 2H), 3.31 (m, 8H), 3.03 (m, 2H), 2.99 (s, 3H), 2.15 (m, 3H), 1.51 (m, 2H).

### REFERENCE EXAMPLE 28

### 2-Ethyl-5,7-dimethyl-3-[2-(4-piperidyl)ethyl]imidazo[4,5-b]pyridine Dihydrochloride

### a) 2-(1-tert-Butoxycarbonylpiperidin-4-yl)ethyl Mesylate

Following a similar procedure to that described in section c of reference example 2, but starting from 2-(1-tert-butoxycarbonylpiperidin-4-yl)ethanol (obtained in section a of reference example 25) and using TEA instead of DIEA and CHCl₃ instead of CH₂Cl₂, the desired compound was obtained in quantitative yield.

### b) 3-[2-(1-tert-Butoxycarbonylpiperidin-4-yl)ethyl]-2-ethyl-5,7-dimethylimidazo[4,5-b]pyridine

Following a similar procedure to that described in section b of reference example 6, but using 2-(1-tert-butoxycarbonylpiperidin-4-yl)ethyl mesylate (obtained in the preceding section) and 3H-2-ethyl-5,7-dimethylimidazo[4,5-b]pyridine (obtained as described in EP 400974) as reagents, instead of 1-tert-butoxycarbonyl-4-(2-chloroethoxycarbonylaminomethyl)piperidine, and carrying out the reaction at room temperature, the desired compound was obtained in 37% yield.

### c) Title Compound

Following a similar procedure to that described in section c of reference example 3, but using 3-[2-(1-tert-butoxycarbonylpiperidin-4-yl)ethyl]-2-ethyl-5,7-dimethylimidazo[4,5-b]pyridine (obtained in the preceding section) as starting product, the title compound of the example was obtained in quantitative yield.

¹H NMR (300 MHz, CD₃OD) δ (TMS): 7.56 (s, 1H), 5.06 (s, 3H), 4.75 (m, 2H), 3.62 (m, 2H), 3.30 (m, 2H), 3.18 (m, 2H), 2.85 (s, 6H), 2.36 (m, 2H), 2.12 (m, 2H), 2.01 (m, 1H), 1.77 (m, 5H).

### REFERENCE EXAMPLE 29

### N-[2-(4-Piperidyl)ethyl]morpholine Dihydrochloride

### a) N-[2-(1-tert-Butoxycarbonylpiperidin-4-yl)ethyl]morpholine

To a mixture of morpholine (0.56 mL, 6.5 mmol), Na₂CO₃ (0.68 g, 6.5 mmol), KI (36 mg) and 2-butanone (50 mL), 2-(1-tert-butoxycarbonylpiperidin-4-yl)ethyl mesylate (obtained in section a of reference example 28) (1 g, 3.25 mmol) was slowly added under argon atmosphere, and the resulting mixture was stirred at reflux overnight. It was filtered and concentrated, to afford a residue which was partitioned between 0.2 M NaHCO₃ and CHCl₃. The organic phase was dried over anhydrous Na₂SO₄ and concentrated. The crude product obtained was purified by chromatography on silica gel using CHCl₃/MeOH 2% as eluent. 0.65 g of the desired compound was obtained (67% yield).

### b) Title Compound

Following a similar procedure to that described in section c of reference example 3, but using N-[2-(1-tert-butoxycarbonylpiperidin-4-yl)ethyl]morpholine (obtained in the preceding section) as starting product, the title compound of the example was obtained in quantitative yield.

¹H NMR (300 MHz, CD₃OD) δ (TMS): 4.83 (s, 3H), 4.03 (m, 2H), 3.85 (m, 2H), 3.50 (m, 2H), 3.39 (m, 2H), 3.25 (m, 4H), 2.99 (m, 2H), 1.98 (broad d, J=14.3 Hz, 2H), 1.80 (m, 3H), 1.49 (m, 2H).

### REFERENCE EXAMPLE 30

### 2-Dimethylamino-N-(4-piperidylmethyl)acetamide Dihydrochloride

### a) N-[(Dimethylamino)acetoxy]succinimide

To a solution of N,N-dimethylglycine hydrochloride (1.95 g, 14 mmol), TEA (3.2 mL, 23 mmol) and NHS (1.61 g, 14 mmol) in CHCl₃ (125 mL), cooled to 0° C. and under argon atmosphere, DCC (2.9 g, 14 mmol) was added, and the resulting mixture was stirred overnight at room temperature. The formed solid was filtered off and the solution was concentrated to dryness. A crude product was obtained which was directly used in the following step.

### b) N-[(1-tert-Butoxycarbonylpiperidin-4-yl)methyl]-2-dimethylaminoacetamide

Following a similar procedure to that described in section a of reference example 3 but using the crude product obtained in the preceding section instead of 4-chlorobutyryl chloride, a crude product was obtained, which was purified by chromatography on silica gel using CHCl₃/MeOH (98:2) as eluent. 2.5 g of the desired compound was obtained.

### c) Title Compound

Following a similar procedure to that described in section c of reference example 3, but using N-[(1-tert-butoxycarbonylpiperidin-4-yl)methyl]-2-dimethylaminoacetamide (obtained in the preceding section) as starting product, the title compound of the example was obtained in quantitative yield.

¹H NMR (300 MHz, CD₃OD) δ (TMS): 4.85 (s, 4H), 4.00 (s, 2H), 3.41 (broad d, J=12.8 Hz, 2H), 3.22 (d, J=6.5 Hz, 2H), 2.97 (m, 2H), 2.94 (s, 6H), 1.98 (m, 3H), 1.47 (m, 2H).

### REFERENCE EXAMPLE 31

### 2-Isopropylamino-1-(4-piperidylmethyl)imidazo[4,5-c]pyridine Trihydrochloride

### a) 1-[(1-tert-Butoxycarbonylpiperidin-4-yl)methyl]-2-isopropylaminoimidazo[4,5-c]pyridine

A mixture of 3-amino-4-[[(1-tert-butoxycarbonylpiperidin-4-yl)methyl]amino]pyridine. (obtained in section b of reference example 8) (5 g, 14.8 mmol) and isopropyl isothiocyanate (1.8 mL, 16.8 mmol) in pyridine (76 mL) was stirred under argon atmosphere at 80° C. for 30 min. EDC.HCl (3.7 g, 19.3 mmol) was added and the resulting mixture was stirred at 90° C. for 2.5 h. It was cooled to room temperature, the solvent was evaporated and the residue was redissolved in EtOAc and H₂O. The phases were separated, the aqueous phase was washed with EtOAc, basified and extracted with EtOAc. The extracted organic phase was dried over sodium sulfate and concentrated to dryness, to afford a crude product which was purified by chromatography on silica gel, using CHCl₃/MeOH mixtures of increasing polarity as eluent. 0.33 g of the desired product was obtained (6% yield).

¹H NMR (300 MHz, CDCl₃) δ (TMS): 8.73 (s, 1H), 8.24 (d, J=5.3 Hz, 1H), 6.98 (d, J=5.3 Hz, 1H), 4.30 (m, 3H), 3.72 (d, J=7.3 Hz, 2H), 3.05 (m, 1H), 2.60 (m, 2H), 1.95 (m, 1H), 1.61 (m, 2H), 1.45 (s, 9H), 1.22 (m, 2H), 1.11 (m, 6H).

### b) Title Compound

Following a similar procedure to that described in section c of reference example 3, but using 1-[(1-tert-butoxycarbonylpiperidin-4-yl)methyl]-2-isopropylaminoimidazo[4,5-c]pyridine (obtained in the preceding section) as starting product, the title compound of the example was obtained in quantitative yield.

### REFERENCE EXAMPLE 32

### 4(Diethylaminomethyl)piperidine Dihydrochloride

### a) 1-(tert-Butoxycarbonyl)-4-(diethylaminomethyl)piperidine

Following a similar procedure to that described in section a of reference example 21 but using acetaldehyde instead of formaldehyde, the desired compound was obtained in 17% yield.

### b) Title Compound

Following a similar procedure to that described in section c of reference example 3, but using 1-(tert-butoxycarbonyl)-4-(diethylaminomethyl)piperidine (obtained in the preceding section) as starting product, the title compound of the example was obtained in quantitative yield.

¹H NMR (300 MHz, CDCl₃+CD₃OD) δ (TMS): 4.29 (s, 3H), 3.36 (m, 2H), 3.22 (m, 4H), 3.11 (m, 4H), 2.22 (m, 1H), 2.12 (m, 2H), 1.64 (m, 2H), 1.37 (m, 6H).

### REFERENCE EXAMPLE 33

### 4-(2-Diethylaminoethyl)piperidine Dihydrochloride

### a) 1-(tert-Butoxycarbonyl)-4-(2-diethylaminoethyl)piperidine

A mixture of 2-(1-tert-butoxycarbonylpiperidin-4-yl)ethyl mesylate (obtained in section a of reference example 28) (0.7 g, 2.4 mmol) and diethylamine (6 mL, 85 mmol) was heated at reflux overnight and under argon atmosphere. It was concentrated to dryness and the residue was redissolved in H₂O and CHCl₃, the phases were separated and the organic phase was dried over sodium sulfate and concentrated to dryness. 0.54 g of the desired product was obtained (79% yield).

### b) Title Compound

Following a similar procedure to that described in section c of reference example 3, but using 1-(tert-butoxycarbonyl)-4-(2-diethylaminoethyl)piperidine (obtained in the preceding section) as starting product, the title compound of the example was obtained in quantitative yield.

¹H NMR (300 MHz, CD₃OD) δ (TMS): 4.79 (s, 3H), 3.38 (broad d, J=10.5 Hz, 2H), 3.30 (m, 2H), 3.25 (m, 4H), 3.00 (m, 2H), 1.99 (broad d, J=13.7 Hz, 2H), 1.74 (m, 3H), 1.54 (m, 2H), 1.33 (t, J=7.1 Hz, 6H).

### REFERENCE EXAMPLE 34

### 4-[2-(1-Pyrrolidinyl)ethyl]piperidine Dihydrochloride

### a) 1-(tert-Butoxycarbonyl)-4-[2-(1-pyrrolidinyl)ethyl]piperidine

Following a similar procedure to that described in section a of reference example 33, but using pyrrolidine instead of diethylamine, the desired compound was obtained in 77% yield.

### b) Title Compound

Following a similar procedure to that described in section c of reference example 3, but using 1-(tert-butoxycarbonyl)₄-[2-(1-pyrrolidinyl)ethyl]piperidine (obtained in the preceding section) as starting product, the title compound of the example was obtained in quantitative yield.

¹H NMR (300 MHz, CD₃OD) δ (TMS): 4.79 (s, 3H), 3.41 (broad d, J=12.8 Hz, 2H), 3.29 (m, 4H), 3.10 (m, 2H), 3.00 (m, 2H), 2.16 (m, 4H), 1.99 (m, 2H), 1.77 (m, 3H), 1.49 (m, 2H).

### REFERENCE EXAMPLE 35

### 4-[2-[(4-Methylpiperazin-1-yl)carbonyloxy]ethyl]piperidine Dihydrochloride

### a) 1-(tert-Butoxycarbonyl)-4-[2-[(4-methylpiperazin-1-yl)carbonyloxy]ethyl]piperidine

Following a similar procedure to that described in section c of reference example 25, but using 1-methylpiperazine instead of pyrrolidine, the desired compound was obtained in 38% yield.

¹H NMR (300 MHz, CDCl₃) δ (TMS): 4.13 (m, 2H), 4.09 (m, 2H), 3.49 (m, 4H), 2.69 (m, 2H), 2.35 (m, 4H), 2.31 (s, 3H), 1.67 (m, 2H), 1.58 (m, 3H), 1.45 (s, 9H), 1.12 (m, 2H)

### b) Desired Compound

Following a similar procedure to that described in section c of reference example 3, but using the compound obtained in the preceding section as starting product, the title compound of the example was obtained in quantitative yield.

### REFERENCE EXAMPLE 36

### 4-[2-[(2-Methoxyethyl)aminocarbonyloxy]ethyl]piperidine Hydrochloride

### a) 1-(tert-Butoxycarbonyl)₄-[2-[(2-methoxyethyl)aminocarbonyloxy]ethyl]piperidine

Following a similar procedure to that described in section c of reference example 25, but using 2-methoxyethylamine instead of pyrrolidine, the desired compound was obtained in 50% yield.

¹H NMR (300 MHz, CDCl₃) δ (TMS): 4.98 (m, 1H), 4.11 (m, 4H), 3.40 (m, 2H), 3.35 (s, 3H), 2.68 (m, 2H), 1.66 (m, 2H), 1.55 (m, 4H), 1.45 (s, 9H), 1.13 (m, 3H).

### b) Title Compound

Following a similar procedure to that described in section c of reference example 3, but using the compound obtained in the preceding section as starting product, the title compound of the example was obtained in quantitative yield.

### REFERENCE EXAMPLE 37

### 4-(1-Pyrrolidinylcarbonylaminomethyl)piperidine Hydrochloride

### a) 1-(tert-Butoxycarbonyl)-4-(phenoxycarbonylaminomethyl)piperidine

Following a similar procedure to that described in section a of reference example 3, but using phenyl chloroformate instead of 4-chlorobutyryl chloride, the desired compound was obtained in 42% yield.

### b) 1-(tert-Butoxycarbonyl)-4-(1-pyrrolidinylcarbonylaminomethyl)piperidine

A mixture of 1-(tert-butoxycarbonyl)-4-(phenoxycarbonylaminomethyl)piperidine (obtained in the preceding section) (1 g, 2.99 mmol) and pyrrolidine (3 mL) was heated overnight at 80° C. It was concentrated to dryness and the residue was treated with CHCl₃ and basified by the addition of 0.5 N NaOH solution. The mixture was treated with 0.2 M NaHCO₃ and CHCl₃, the phases were separated and the organic phase was dried over sodium sulfate and concentrated. The crude product obtained was purified by chromatography on silica gel using EtOAc/MeOH mixtures of increasing polarity as eluent, to afford 780 mg of the desired compound (84% yield).

### c) Title Compound

Following a similar procedure to that described in section c of reference example 3, but using the compound obtained in the preceding section as starting product, the title compound of the example was obtained in quantitative yield.

¹H NMR (300 MHz, DMSO-d₆) δ (TMS): 8.78 (s, 1H), 8.49 (s, 1H), 6.15 (s, 1H), 3.41 (m, 2H), 3.14 (m, 4H), 2.90 (d, J=6.2 Hz, 2H), 2.74 (m, 2H), 1.77 (m, 6H), 1.64 (m, 1H), 1.30 (m, 2H).

### REFERENCE EXAMPLE 38

### 4-(4-Morpholinylcarbonylaminomethyl)piperidine Hydrochloride

### a) 1-(tert-Butoxycarbonyl)-4-4-morpholinylcarbonylaminomethyl)piperidine

Following a similar procedure to that described in section b of reference example 37, but using morpholine instead of pyrrolidine, the desired compound was obtained in 97% yield.

### b) Title Compound

Following a similar procedure to that described in section c of reference example 3, but using the compound obtained in the preceding section as starting product, the title compound of the example was obtained in quantitative yield.

¹H NMR (300 MHz, DMSO-d₆) δ (TMS): 8.84 (s, 1H), 8.58 (s, 1H), 6.64 (s, 1H), 3.40 (m, 6H), 3.22 (m, 4H), 2.91 (d, J=5.3 Hz, 2H), 2.78 (m, 2H), 1.69 (m, 3H), 1.28 (m, 2H).

### REFERENCE EXAMPLE 39

### 4-(1-Piperidylmethyl)piperidine Dihydrochloride

### a) 1 (tert-Butoxycarbonyl)-4-(1-piperidylmethyl)piperidine

Following a similar procedure to that described in section a of reference example 29, but using piperidine instead of morpholine and (1-tert-butoxycarbonylpiperidin-4-yl)methyl mesylate (obtained in reference example 2) instead of 2-(1-tert-butoxycarbonylpiperidin-4-yl)ethyl mesylate, the desired compound was obtained in quantitative yield.

### b) Title Compound

Following a similar procedure to that described in section c of reference example 3, but using 1-(tert-butoxycarbonyl)-4-(1-piperidylmethyl)piperidine (obtained in the preceding section) as starting product, the title compound of the example was obtained in quantitative yield.

¹H NMR (300 MHz, DMSO-d₆) δ (TMS): 10.23 (s, 1H), 9.00 (s, 1H), 8.89 (s, 1H), 3.45 (m, 2H), 3.25 (m, 2H), 2.90 (m, 2H), 2.85 (m, 4H), 2.11 (m, 1H), 1.93 (m, 4H), 1.7 (m, 4H), 1.45 (m, 2H).

### REFERENCE EXAMPLE 40

### N-Ethyl-2,2,2-trifluoro-N-(4-piperidylmethyl)acetamide Hydrochloride

### a) N-[(1-tert-Butoxycarbonylpiperidin-4-yl)methyl]-2,2,2-trifluoroacetamide

To a solution of ethyl trifluoroacetate (3.33 mL, 28 mmol) in tetrahydrofuran (3 mL) under argon atmosphere, 4-aminomethyl-1-tert-butoxycarbonylpiperidine (obtained in reference example 1). (6 g, 28 mmol) was added, and the resulting mixture was stirred overnight at room temperature. It was concentrated to dryness and 7.1 g of a crude product was obtained, which was directly used in the following reaction.

### b) N-[(1-tert-Butoxycarbonylpiperidin-4-yl)methyl]-N-ethyl-2,2,2-trifluoroacetamide

Following a similar procedure to that described in section b of reference example 6, but using ethyl iodide and N-[(1-tert-butoxycarbonylpiperidin-4-yl)methyl]-2,2,2-trifluoroacetamide (obtained in the preceding section) as reagents, instead of 1-tert-butoxycarbonyl-4-(2-chloroethoxycarbonylaminomethyl)piperidine, the desired compound was obtained in 37% yield.

¹H NMR (300 MHz, CDCl₃) δ (TMS): 4.10 (q, J=6.8 Hz, 2H), 4.02 (m, 2H), 3.26 (m, 2H), 2.65 (broad t, J=12.2 Hz, 2H), 1.98 (m, 1H), 1.43 (m, 2H), 1.42 (s, 9H), 1.18 (t, J=6.8 Hz, 3H), 1.16 (m, 2H).

### c) Title Compound

Following a similar procedure to that described in section c of reference example 3, but using the compound obtained in the preceding section as starting product, the title compound of the example was obtained in quantitative yield.

### REFERENCE EXAMPLE 41

### 4-[(4-Methylpiperazin-1-yl)carbonylaminomethyl]piperidine Dihydrochloride

### a) 1-(tert-Butoxycarbonyl)-4-[(4-methylpiperazin-1-yl)carbonylaminomethyl]piperidine

Following a similar procedure to that described in section b of reference example 37, but using 1-methylpiperazine instead of pyrrolidine, the desired compound was obtained in 53% yield.

### b) Title Compound

Following a similar procedure to that described in section c of reference example 3, but using the compound obtained in the preceding section as starting product, the title compound of the example was obtained in quantitative yield.

¹H NMR (300 MHz, DMSO-d₆) δ (TMS): 10.92 (s, 1H), 8.84 (s, 1H), 8.63 (s, 1H), 6.90 (s, 1H), 4.03 (broad d, J=14.3 Hz, 2H), 3.39 (m, 4H), 3.18 (m, 4H), 2.85 (m, 4H), 2.71 (s, 3H), 1.69 (m, 3H), 1.26 (m, 2H).

### REFERENCE EXAMPLE 42

### 4-(4-Pyridylaminomethyl)piperidine Dihydrochloride

### a) 1-(tert-Butoxycarbonyl)4-(4-pyridylaminomethyl)piperidine

To a solution of 4-chloropyridine (2 g, 13.3 mmol) and TEA (5.6 mL, 40.2 mmol) in xylene (25 mL), a solution of 4-aminomethyl-1-tert-butoxycarbonylpiperidine (obtained in reference example 1) (2.86 g, 13.3 mmol) in xylene (5 mL) was added dropwise under argon atmosphere, and the resulting mixture was heated at reflux for 3 days. The solvent was removed and the residue was treated with CHCl₃ and 0.5 N NaOH solution. The phases were separated and the combined organic phases were dried over sodium sulfate and concentrated to dryness. The crude product obtained was purified by chromatography on silica gel using a CHCl₃/MeOH/NH₃ 100:10:1 mixture as eluent. 200 mg of the desired compound was obtained.

¹H NMR (300 MHz, CDCl₃) δ (TMS): 8.18 (d, J=6 Hz, 2H), 7.30 (m, 1H), 6.41 (d, J=6 Hz, 2H), 4.16 (m, 2H), 3.06 (m, 2H), 2.69 (broad t, J=12.6 Hz, 2H), 1.74 (m, 3H), 1.46 (s, 9H), 0.85 (m, 2H).

### b) Title Compound

Following a similar procedure to that described in section c of reference example 3, but using 1-(tert-butoxycarbonyl)4-(4-pyridylaminomethyl)piperidine (obtained in the preceding section) as starting product, the title compound of the example was obtained in quantitative yield.

### REFERENCE EXAMPLE 43

### Isobutyl N-ethyl-N-(4-piperidylmethyl)carbamate Hydrochloride

### a) 1-(tert-Butoxycarbonyl)4-(ethylaminomethyl)piperidine

To a solution of N-[(1-tert-butoxycarbonylpiperidinyl)methyl]-N-ethyl-2,2,2-trifluoroacetamide (obtained in section b of reference example 40) (700 mg, 2.0 mmol) in EtOH (10 mL), 1 N NaOH solution (10 mL) was added, and the resulting mixture was stirred overnight at room temperature. EtOH was removed and the resulting suspension was extracted 3 times with CHCl₃. The combined organic phases were dried over sodium sulfate and concentrated to dryness, to afford a crude product which was directly used in the following step.

### b) Isobutyl N-[(1-tert-butoxycarbonylpiperidin-4-yl)methyl]-N-ethylcarbamate

Following a similar procedure to that described in section a of reference example 3, but using 1-(tert-butoxycarbonyl)*4-(ethylaminomethyl)piperidine (obtained in the preceding section) and isobutyl chloroformate as reagents and treating the residue with 2.5% NaHSO₄ solution instead of 0.2 M NaHCO₃ solution, the desired compound was obtained in 82% yield.

### c) Title Compound

Following a similar procedure to that described in section c of reference example 3, but using isobutyl N-[(1-tert-butoxycarbonylpiperidin-4-yl)methyl]-N-ethylcarbamate (obtained in the preceding section) as starting product, the title compound of the example was obtained in quantitative yield.

¹H NMR (300 MHz, CDCl₃) δ (TMS): 9.65 (broad s, 1H), 9.35 (broad s, 1H), 3.85 (d, J=6.5 Hz, 2H), 3.50 (m, 2H), 3.26 (m, 2H), 3.13 (d, J=6.3 Hz, 2H), 2.85 (m, 2H), 1.92 (m, 4H), 1.25 (m, 2H) 1.12 (m, 3H), 0.93 (d, J=6.6 Hz, 6H).

### REFERENCE EXAMPLE 44

### 4-Piperidylmethyl N,N-diethylcarbamate Hydrochloride

### a) 1-tert-Butoxycarbonyl-4-(phenoxycarbonyloxymethyl)piperidine

Following a similar procedure to that described in section a of reference example 3, but using (1-tert-butoxycarbonylpiperidin-4-yl)methanol (obtained in section b of reference example 2) instead of 4-aminomethyl-1-tert-butoxycarbonylpiperidine and using phenyl chloroformate instead of 4-chlorobutyryl chloride, the desired compound was obtained in 26% yield.

### b) [1-(tert-Butoxycarbonyl)piperidin-4-yl]methyl N,N-diethylcarbamate

Following a similar procedure to that described in section c of reference example 25, but starting from the compound obtained in the preceding section instead of 1-tert-butoxycarbonyl-4-[2-(phenoxycarbonyloxy)ethyl]piperidine and using diethylamine instead of pyrrolidine, the desired compound was obtained in 66% yield.

### c) Title Compound

Following a similar procedure to that described in section c of reference example 3, but using [1-(tert-butoxycarbonyl)piperidin-4-yl]methyl N,N-diethylcarbamate (obtained in the preceding section) as starting product, the title compound of the example was obtained in quantitative yield.

¹H NMR (300 MHz, CDCl₃) δ (TMS): 9.74 (broad s, 1H), 9.45 (broad s, 1H), 3.99 (d, J=5.3 Hz, 2H), 3.52 (broad d, J=11.4 Hz, 2H), 3.27 (m, 4H), 2.86 (broad d, J=9.9 Hz, 2H), 1.94 (m, 1H), 1.90 (m, 2H), 1.75 (m, 2. H), 1.12 (m, 6H).

### REFERENCE EXAMPLE 45

### 4-[[(4-Methylpiperazin-1-yl)carbonyloxy]methyl]piperidine Dihydrochloride

### a) 1-(tert-Butoxycarbonyl)-4-[[(4-methylpiperazin-1-yl)carbonyloxy]methyl]piperidine

Following a similar procedure to that described in section c of reference example 25, but starting from 1-tert-butoxycarbonyl-4-(phenoxycarbonyloxymethyl)piperidine (obtained in section a of reference example 44) and using 1-methylpiperazine instead of pyrrolidine, the desired compound was obtained in 79% yield.

¹H NMR (300 MHz, CDCl₃) δ (TMS): 4.12 (broad d, J=11.9 Hz, 2H), 3.95 (d, J=6.3 Hz, 2H), 3.50 (m, 4H), 2.70 (broad t, J=11.8 Hz, 2H), 2.38 (m, 4H), 2.30 (s, 3H), 1.81 (m, 1H), 1.68 (m, 2H), 1.45 (s, 9H), 1.17 (m, 2H).

### b) Title Compound

Following a similar procedure to that described in section c of reference example 3, but using the compound obtained in the preceding section as starting product, the title compound of the example was obtained in quantitative yield.

### REFERENCE EXAMPLE 46

### 4-(4-Pyridyloxymethyl)piperidine Dihydrochloride

### a) 1-(tert-Butoxycarbonyl)-4-(4-pyridyloxymethyl)piperidine

To a suspension of 4-hydroxypyridine (1.3 g, 14 mmol), (1-tert-butoxycarbonylpiperidin-4-yl)methanol (3 g, 14 mmol) (obtained in section b of reference example 2) and triphenylphosphine (3.66 g, 14 mmol) in THF (40 mL), cooled to −20° C., a solution of DEAD (2.2 mL, 14 mmol) in THF (12 mL) was added dropwise and under argon atmosphere, and the resulting mixture was stirred overnight at room temperature. THF was evaporated and the resulting residue was resuspended in H₂O and extracted 3 times with EtOAc. The combined organic extracts were dried over Na₂SO₄ and concentrated to dryness, to afford a crude product, which was purified by chromatography on silica gel using EtOAc/MeOH mixtures of increasing polarity as eluent. 1.15 g of the desired compound was obtained (28% yield).

### b) Title Compound

Following a similar procedure to that described in section c of reference example 3, but using the compound obtained in the preceding section as starting product, the title compound of the example was obtained in quantitative yield.

¹H NMR (300 MHz, CD₃OD) δ (TMS): 8.66 (d, J=7.5 Hz, 2H), 7.57 (d, J=7.5 Hz, 2H), 4.90 (s, 3H), 4.31 (d, J=6.1 Hz, 2H), 3.48 (m, 2H), 3.08 (m, 2H), 2.30 (m, 1H), 2.10 (m, 2H), 1.70 (m, 2H).

### REFERENCE EXAMPLE 47

### N-(3,5-Dichlorophenylsulfonyl)-L-proline

To a solution of Na₂CO₃ (10.79 g, 101.85 mmol) in H₂O (50 mL), (L)-proline (3.9 g, 33.9 mmol) was added. The mixture was cooled to 0° C. and a suspension of 3,5-dichlorobenzenesulfonyl chloride (10.0 g, 40.7 mmol) in dioxane (20 mL) was slowly added. The resulting mixture was stirred at 0° C. for 30 min, H₂O (15 mL) was added and the mixture was stirred overnight at room temperature. Dioxane was evaporated, the aqueous phase was diluted with H₂O and extracted with EtOAc (×3). It was cooled to 0° C. and brought to pH 1.5 by the addition of 3 N HCl solution (50 mL). An insoluble material was obtained, which was collected by filtration and was washed with H₂O. The aqueous phase was extracted with EtOAc. The organic phase was dried over sodium sulfate and concentrated, and the product obtained was combined with the previous solid. 9.74 g of the title compound was obtained (89% yield).

¹H NMR (300 MHz, CDCl₃) δ (TMS): 9.84 (broad s, 1H), 7.75 (broad s, 2H), 7.55 (s, 1H), 4.40 (m, 1H), 3.50 (m, 1H), 3.36 (m, 1H), 1.9-2.20 (complex signal, 4H).

### REFERENCE EXAMPLE 48

### 1-Methyl N-[1-(3,5-dichlorophenylsulfonyl)-L-prolyl]-L-glutamate

### a) N-[[N′-(3,5-dichlorophenylsulfonyl)-L-prolyl]oxy]succinimide

To a solution of N-(3,5-dichlorophenylsulfonyl)-L-proline (obtained in reference example 47) (3.6 g, 11.1 mmol) and NHS (1.27 g, 11.1 mmol) in CHCl₃ (125 mL), cooled to 0° C. and under argon atmosphere, DCC (2.27 g, 11.1 mmol) was added and the resulting mixture was stirred overnight at room temperature. The solid obtained was filtered off and the solution was concentrated to dryness. A crude product was obtained which was directly used in the following step.

### b) Title Compound

N-[[N′-(3,5-dichlorophenylsulfonyl)-L-prolyl]oxy]succinimide (prepared in the preceding section) (4.67 g, 11.1 mmol) was dissolved in CHCl₃ (60 mL). TEA (3.25 mL, 22.2 mmol) was added and then 1-methyl L-glutamate (1.81 g, 11.1 mmol) was slowly added portionwise. The solution was stirred overnight at room temperature, acidified with 0.5 N HCl (pH 4) and the product was extracted with CHCl₃. The crude product obtained was purified by chromatography on silica gel using EtOAc as eluent, to afford 3.1 g of the title compound of the example (60% yield).

¹H NMR (300 MHz, CDCl₃) δ (TMS): 7.75 (s, 2H), 7.62 (s, 1H), 7.35 (d, J=7.6 Hz, 1H), 4.62 (m, 1H), 4.16 (m, 1H), 3.79 (s, 3H), 3.59 (m, 1H), 3.23 (m, 1H), 2.46 (m, 2H), 2.23 (m, 2H), 2.07 (m, 1H), 1.75-1.85 (complex signal, 3H).

### REFERENCE EXAMPLE 49

### 1-Methyl N-[1-tosyl-L-prolyl]-L-glutamate

Following a similar procedure to that described in reference example 48 but using N-tosyl-L-proline instead of N-(3,5-dichlorophenylsulfonyl)-proline, the title compound of the example was obtained in 90% yield.

¹H NMR (300 MHz, CDCl₃) δ (TMS): 7.74 (d, J=8.3 Hz, 2H), 7.55 (d, J=7.8 Hz, 1H), 7.34 (d, J=8.3 Hz, 2H), 4.61 (m, 1H), 4.12 (m, 1H), 3.78 (s, 3H), 3.52 (m, 1H), 3.17 (m, 1H), 2.44 (s, 3H), 2.42 (m, 2H), 2.01 (m, 2H), 1.95 (m, 1H), 1.78 (m, 1H), 1.62 (m, 2H).

### REFERENCE EXAMPLE 50

### 1-Methyl N-tert-butoxycarbonyl-L-glutamate

To a suspension of 1-methyl L-glutamate (5.0 g, 30 mmol) in CH₂Cl₂ (250 mL) and TEA (8.64 mL, 60 mmol), cooled to 0° C., a solution of di-tert-butyl dicarbonate (6.7 g, 30 mmol) in CH₂Cl₂ (40 mL) was added dropwise and under argon atmosphere. The mixture was stirred overnight at room temperature. The resulting mixture was brought to pH 34 by addition of 10% NaHSO₄ solution and next it was extracted 3 times with CH₂Cl₂. The combined organic phases were dried over sodium sulfate and concentrated, to afford the title compound of the example in quantitative yield.

¹H NMR (300 MHz, CDCl₃) δ (TMS): 5.30 (m, 1H), 4.31 (m, 1H), 3.74 (s, 3H), 2.41 (m, 2H), 2.14 (m, 1H), 1.98 (m, 1H), 1.45 (s, 9H).

### REFERENCE EXAMPLE 51

Methyl (2S)-2-amino-5-[4-(2-methylimidazo[4,5-c]pyridin-1-ylmethyl)piperidin-1-yl]-5-oxopentanoate Dihydrochloride

### a) Methyl (2S)-2-(tert-butoxycarbonylamino)-5-[4-(2-methylimidazo[4,5-c]pyridin-1-yl-methyl)piperidin-1-yl]-5-oxopentanoate

A solution of the compound obtained in reference example 50 (3.8 g, 15 mmol), DCC (2.9 g, 14 mmol) and HOBT (1.9 g, 14 mmol) in DMF (50 mL) was stirred at room temperature and under argon atmosphere for 1 hour. Next, 2-methyl-1-(4-piperidylmethyl)imidazo[4,5-c]pyridine (obtained in reference example 8) (3.35 g, 15 mmol) was added, and the resulting mixture was stirred overnight at room temperature. The solid obtained was filtered off and the solution was concentrated to dryness. The residue obtained was partitioned between CHCl₃ and saturated NaHCO₃ solution. The phases were separated and the organic phase was dried over sodium sulfate and concentrated, to afford a crude product which was purified by chromatography on silica gel using a CHCl₃:MeOH 5% mixture as eluent. 5.41 g of the desired compound was obtained (73% yield).

### b) Title Compound

Following a similar procedure to that described in section c of reference example 3, but using the compound obtained in the preceding section as starting product, the title compound of the example was obtained in quantitative yield.

¹H NMR (300 MHz, DMSO-d₆) δ (TMS): 9.74 (broad s, 4H), 9.32 (s, 1H), 8.62 (d, J=6.5 Hz, 1H), 8.33 (d, J=6.5 Hz, 1H), 4.31 (d, J=7.5 Hz, 2H), 4.30 (m, 1H), 4.00 (m, 1H), 3.80 (m, 1H), 3.70 (s, 3H), 2.88 (m, 1H), 2.75 (s, 3H), 2.41 (m, 3H), 1.98-2.06 (complex signal, 3H), 1.15-1.52 (complex signal, 4H).

### REFERENCE EXAMPLE 52

### Methyl (2S)-3-amino-2-[1-(3,5-dichlorophenylsulfonyl)-L-prolyl]aminopropionate Hydrochloride

### a) Methyl (2S)-3-tert-butoxycarbonylamino-2-[1(3,5-dichlorophenylsulfonyl)-L-prolyl]aminopropionate

Following a similar procedure to that described in reference example 48 but using methyl (2S)-2-amino-3-tert-butoxycarbonylaminopropionate hydrochloride instead of 1-methyl L-glutamate, the title compound of the example was obtained in 98% yield.

### b) Title Compound

Following a similar procedure to that described in section c of reference example 3, but using methyl (2S)-3-tert-butoxycarbonylamino-2-[1-(3,5-dichlorophenylsulfonyl)-L-prolyl]aminopropionate (obtained in the preceding section) as starting product, the title compound of the example was obtained in quantitative yield.

¹H NMR (300 MHz, CDCl₃+CD₃OD) δ (TMS): 7.79 (s, 2H), 7.59 (s, 1H), 4.71 (m, 1H), 4.07 (m, 1H), 3.76 (m, 1H), 3.75 (s, 3H), 3.67 (s, 4H), 3.44 (m, 2H), 3.16 (m, 1H), 2.05 (m, 4H).

### REFERENCE EXAMPLE 53

### Methyl (2S)-2-amino-3-[[4-dimethylaminomethyl)piperidin-1-yl]carbonyloxy]propionate

### a) N-tert-butoxycarbonyl-L-serine Methyl Ester

Following a similar procedure to that described in section a of reference example 3 but using L-serine methyl ester hydrochloride and di-tert-butyl dicarbonate as reagents, 6.19 g of the title compound was obtained (88% yield).

### b) Methyl (2S)-2-(tert-butoxycarbonylamino)-3-[(1-imidazolyl)carbonyloxy]propionate

To a solution of 1,1′-carbonyldiimidazole (0.74 g, 4.56 mmol) in acetonitrile (15 mL), the compound obtained in the preceding section (1.0 g, 4.56 mmol) dissolved in 8 mL of acetonitrile was added dropwise and under argon atmosphere, and the resulting mixture was stirred overnight at room temperature. The solvent was removed and the residue obtained was treated with H₂O and CHCl₃. The phases were separated, the organic phase was dried over sodium sulfate and concentrated to dryness. 1.10 g of the desired compound was obtained (77% yield).

### c) Methyl (2S)-2-(tert-butoxycarbonylamino)-3-[[4-(dimethylaminomethyl)piperidin-1-yl]carbonyloxy]propionate

To a suspension of 4-(dimethylaminomethyl)piperidine dihydrochloride (obtained in reference example 21) (0.36 g, 1.67 mmol) and DIEA (1.2 mL, 6.9 mmol) in acetonitrile (10 mL), some drops of 2 N NaOH solution and some drops of H₂O were added until complete dissolution. Next, methyl (2S)-2-(tert-butoxycarbonylamino)-3-[(1-imidazolyl)carbonyloxy]propionate (obtained in the preceding section) (7.3 mL, 1.67 mmol) was slowly added and the resulting mixture was stirred overnight at room temperature. Acetonitrile was evaporated and the residue obtained was treated with CHCl₃ and saturated NaHCO₃ solution. The phases were separated, the organic phase was dried over sodium sulfate and concentrated to dryness. The crude product obtained was purified on silica gel, using CHCl₃/MeOH mixtures of increasing polarity as eluent. 77 mg of the desired compound was obtained (12% yield).

¹H NMR (300 MHz, CDCl₃) δ (TMS): 5.39 (d, J=7.8 Hz, 1H), 4.51 (m, 1H), 4.27 (m, 2H), 4.08 (m, 1H), 3.96 (m, 1H), 3.72 (s, 3H), 2.71 (m, 2H), 2.16 (s, 6H), 2.07 (d, J=7.1 Hz, 2H), 1.70 (broad d, J=13.1 Hz, 2H), 1.62 (m, 1H), 1.41 (s, 9H), 1.10 (m, 2H).

d) Title Compound

Following a similar procedure to that described in section c of reference example 3, but using methyl (2S)-2-(tert-butoxycarbonylamino)-3-[[4-(dimethylaminomethyl)piperidin-1-y]carbonyloxy]propionate (obtained in the preceding section) as starting product, the title compound of the example was obtained in quantitative yield.

### REFERENCE EXAMPLE 54

### Methyl (2S)-3-amino-2-[N-tosyl-L-prolyl]aminopropionate

### a) Methyl (2S)-3-tert-butoxycarbonylamino-2-[N-tosyl-L-prolyl]aminopropionate

Following a similar procedure to that described in reference example 48 but using N-tosyl-L-proline instead of N-(3,5-dichlorophenylsulfonyl)-L-proline and methyl (2S)-2-amino-3-tert-butoxycarbonylaminopropionate hydrochloride instead of 1-methyl L-glutamate, the title compound of the example was obtained in 81% yield.

### b) Title Compound

The compound obtained in the preceding section and a 4 M dioxane/HCl_{(g)} mixture were mixed in a flask under argon atmosphere. The mixture was stirred overnight at room temperature and concentrated to dryness. The residue obtained was partitioned between aqueous NaHCO₃ solution and CHCl₃. The phases were separated and the organic phase was dried over sodium sulfate and concentrated to dryness, to afford the title compound of the example in quantitative yield.

¹H NMR (300 MHz, CDCl₃) δ (TMS): 7.74 (d, J=8.3 Hz, 2H), 7.62 (d, J=7.2 Hz, 1H), 7.33 (d, J=8.3 Hz, 2H), 4.55 (m, 1H), 4.13 (m, 1H), 3.69 (s, 3H), 3.52 (m, 1H), 3.20 (m, 1H), 3.10 (m, 2H), 2.44 (s, 3H), 2.17 (m, 1H), 1.67 (m, 1H), 1.60 (m, 2H), 1.38 (s, 2H).

### REFERENCE EXAMPLE 55

### 1-(4-Piperidylmethyl)-2-propylimidazo[4,5-c]pyridine Dihydrochloride

### a) 1-[(1-tert-Butoxycarbonylpiperidin-4-yl)methyl]-2-propylimidazo[4,5-c]pyridine

A solution of 3-amino-4-[[(1-tert-butoxycarbonylpiperidin-4-yl)methyl]amino]pyridine (obtained in section b of reference example 8) (3.6 g, 11.7 mmol) in butyric anhydride (15.5 mL) and butyric acid (16.5 mL) was heated under argon atmosphere at 100° C. for 4 h., and at 120° C. for 1 h more. The resulting mixture was poured over ice, brought to basic pH by the addition of 5% NaOH solution and stirred for 30 min. The resulting suspension was extracted 3 times with CH₂Cl₂. The combined organic phases were dried over sodium sulfate and concentrated to dryness, to afford a crude product which was purified by chromatography on silica gel, using a CHCl₃/MeOH (97:3) mixture as eluent. 1.28 g of the desired compound was obtained (32% yield).

¹H NMR (300 MHz, CDCl₃) δ (TMS): 9.03 (s, 1H), 8.39 (d, J=5.5 Hz, 1H), 7.23 (d, J=5.5 Hz, 1H), 4.14 (m, 2H), 3.98 (d, J=8.2 Hz, 2H), 2.84 (t, J=7.5 Hz, 2H), 2.61 (broad t, J=12.2 Hz, 2H), 1.95 (q, J=7.5 Hz, 2H), 1.66 (m, 3H), 1.45 (s, 9H), 1.27(m, 2H), 1.09 (t, J=7.5 Hz, 3H).

### b) Title Compound

Following a similar procedure to that described in section c of reference example 3, but using the compound obtained in the preceding section as starting product, the title compound of the example was obtained in quantitative yield.

¹H NMR (300 MHz, CD₃OD) δ (TMS): 8.99 (s, 1H), 8.48 (s, 1H), 7.43 (s, 1H), 4.36 (m, 5H), 3.30 (m, 2H), 3.00 (t, J=7.5 Hz, 2H), 2.90 (broad t, J=12.5 Hz, 2H), 2.25 (m, 1H), 2.00 (q, J=7.5 Hz, 2H), 1.93 (m, 2H), 1.67 (m, 2H), 1.10 (q, J=7.5 Hz, 3H).

### REFERENCE EXAMPLE 56

### 2-(4-Piperidyl)ethyl 4-phenylpiperazine-1-carboxylate Dihydrochloride

### a) 2-(1-tert-Butoxycarbonylpiperidin-4-yl)ethyl 4-phenylpiperazine-1-carboxylate

Following a similar procedure to that described in section b of reference example 37, but using 1-tert-butoxycarbonyl-4-[2-(phenoxycarbonyloxy)ethyl]piperidine (obtained in section b of reference example 25) and 1-phenylpiperazine as starting products, the desired compound was obtained in quantitative yield.

¹H NMR (300 MHz, CDCl₃) δ (TMS): 7.28 (m, 2H), 6.87 (m, 3H), 4.14 (m, 4H), 3.63 (m, 4H), 3.14 (m, 4H), 2.69 (broad t, J=12.2 Hz, 2H), 1.5-1.7 (m, 5H), 1.48 (s, 9H), 1.17 (m, 2H).

### b) Title Compound

Following a similar procedure to that described in section c of reference example 3, but using the compound obtained in the preceding section as starting product, the title compound of the example was obtained in quantitative yield.

### REFERENCE EXAMPLE 57

Methyl (4S)-3-[1-(3,5-dichlorophenylsulfonyl)-L-prolyl]-2-oxoimidazolidine-4-carboxylate

### a) 1-Methyl N-[1-(3,5-dichlorophenylsulfonyl)-L-prolyl)]-L-aspartate

Following a similar procedure to that described in reference example 48 but using 1-methyl L-aspartate instead of 1-methyl L-glutamate, the desired compound was obtained in 94% yield.

¹H NMR (300 MHz, CDCl₃) δ (TMS): 7.66 (s, 2H), 7.51 (s, 1H), 7.50 (m, 1H), 4.75 (m, 1H), 4.08 (m, 1H), 3.70 (s, 3H), 3.48 (m, 1H), 3.11 (m, 1H), 2.93 (qd, J=8.7 Hz, J=4.8 Hz, 2H), 2.10 (m, 1H), 1.72 (m, 3H).

### b) Title Compound

To a solution of the compound obtained in the preceding section (0.44 g, 0.97 mmol) and TEA (0.135 mL, 0.96 mmol) in benzene (10 mL), diphenylphosphoryl azide (0.221 mL, 1 mmol) was added under argon atmosphere, and the reaction mixture was stirred at room temperature for 30 min. and next at reflux overnight. The resulting mixture was partitioned three times between H₂O and EtOAc and the combined organic phases were dried over sodium sulfate and concentrated. The title compound of the example was obtained in quantitative yield.

¹H NMR (300 MHz, CDCl₃) δ (TMS): 7.77 (s, 2H), 7.55 (s, 1H), 7.35 (m, 1H), 5.72 (m, 1H), 4.95 (m, 1H), 3.77 (s, 3H), 3.55 (m, 2H), 3.34 (m, 1H), 3.01 (m, 1H), 2.22 (m, 1H), 2.12 (m, 1H), 1.95 (m, 1H), 1.86 (m, 1H).

### REFERENCE EXAMPLE 58

### 2-(4-Piperidyl)ethyl 4-(4-pyridyl)piperazine-1-carboxylate

### a) 2-(1-tert-Butoxycarbonylpiperidin-4-yl)ethyl 4-(4-pyridyl)piperazine-1-carboxylate

Following a similar procedure to that described in section c of reference example 25, but using 1-tert-butoxycarbonyl-4-[2-(phenoxycarbonyloxy)ethyl)piperidine (obtained in section b of reference example 25) and 1-(4-pyridyl)piperazine as starting products, the desired compound was obtained in 89% yield.

¹H NMR (300 MHz, CDCl₃) δ (TMS): 8.21 (d, J=4.8 Hz, 2H), 6.64 (d, J=4.8 Hz, 2H), 4.14 (t, J=6.3 Hz, 2H), 4.04 (m, 4H), 3.59 (m, 4H), 3.37 (m, 4H), 2.60 (broad t, J=12.3 Hz, 2H), 1.5-1.7 (m, 5H), 1.43 (s, 9H), 1.13 (m, 2H).

### b) Title Compound

Following a similar procedure to that described in section b of reference example 7, but using 2-(1-tert-butoxycarbonylpiperidin-4-yl)ethyl 4-(4-pyridyl)piperazine-1-carboxylate (obtained in the preceding section) as starting product, the title compound of the example was obtained in 75% yield.

¹H NMR (300 MHz, CDCl₃) δ (TMS): 8.60 (d, J=4.8 Hz, 2H), 6.65 (d, J=4.8 Hz, 2H), 4.16 (t, J=6.3 Hz, 2H), 3.62 (m, 4H), 3.33 (m, 4H), 3.06 (m, 2H), 2.60 (td, J=12.3 Hz, J=2.5 Hz, 2H), 1.5-1.7 (m, 6H), 1.13 (m, 2H).

### REFERENCE EXAMPLE 59

### 4-[2-[(4-Ethoxycarbonylpiperazin-1-yl)carbonyloxy]ethyl]piperidine

### a) 1-(tert-Butoxycarbonyl)-4-[2-[(4-ethoxycarbonylpiperazin-1-yl)carbonyloxy]ethyl]piperidine

Following a similar procedure to that described in section b of reference example 37, but using 1-tert-butoxycarbonyl-4-[2-(phenoxycarbonyloxy)ethyl]piperidine (obtained in section b of reference example 25) instead of 1-(tert-butoxycarbonyl)-4-(phenoxycarbonylaminomethyl)piperidine and ethyl 1-piperazinecarboxylate instead of pyrrolidine, the desired compound was obtained in quantitative yield.

¹H NMR (300 MHz, CDCl₃) δ (TMS): 4.13 (m, 6H), 3.45 (s, 8H), 2.67 (broad t, J=12.3 Hz, 2H), 1.5-1.7 (m, 5H), 1.44 (s, 9H), 1.25 (t, J=4 Hz, 3H), 1.15 (m, 2H).

### b) Title Compound

Following a similar procedure to that described in section b of reference example 7, but using the compound obtained in the preceding section as starting product, the title compound of the example was obtained in 98% yield.

¹H NMR (300 MHz, CDCl₃) δ (TMS): 4.17 (m, 4H), 3.45 (s, 8H), 3.04 (m, 2H), 2.67 (broad t, J=12.3 Hz, 2H), 1.5-1.7 (m, 6H), 1.25 (t, J=4 Hz, 3H), 1.15 (m, 2H).

### REFERENCE EXAMPLE 60

### 2-(4-Piperidyl)ethyl 4-methylpiperidine-1-carboxylate

### a) 2-(1-tert-Butoxycarbonylpiperidinyl)ethyl 4-methylpiperidine-1-carboxylate

Following a similar procedure to that described in section b of reference example 37, but using 1-tert-butoxycarbonyl-4-[2-(phenoxycarbonyloxy)ethyl]piperidine (obtained in section b of reference example 25) instead of 1-(tert-butoxycarbonyl)-4-(phenoxycarbonylaminomethyl)piperidine and 4-methylpiperidine instead of pyrrolidine, the desired compound was obtained in 98% yield.

¹H NMR (300 MHz, CDCl₃) δ (TMS): 4.11 (m, 6H), 2.69 (broad q, J=13.2 Hz, 4H), 1.5-1.7 (m, 6H), 1.45 (s, 9H), 1.15 (m, 6H), 0.93 (d, J=6.6 Hz, 3H).

### b) Title Compound

Following a similar procedure to that described in section b of reference example 7, but using the compound obtained in the preceding section as starting product, the title compound of the example was obtained in 93% yield.

¹H NMR (300 MHz, CDCl₃) δ (TMS): 4.11 (m, 4H), 3.08 (m, 2H), 2.72 (broad t, J=12.3 Hz, 2H), 2.57 (td, J=12.3 Hz, J=2.7 Hz, 2H), 1.5-1.7 (m, 7H), 1.15 (m, 6H), 0.94 (d, J=6.6 Hz, 3H).

### REFERENCE EXAMPLE 61

### 2-(4-Piperidyl)ethyl 4-methylhomopiperazine-1-carboxylate

### a) 2-(1-tert-Butoxycarbonylpiperidin-4-yl)ethyl 4-methylhomopiperazine-1-carboxylate

Following a similar procedure to that described in section b of reference example 37, but using 1-tert-butoxycarbonyl-4-[2-(phenoxycarbonyloxy)ethyl]piperidine (obtained in section b of reference example 25) instead of 1-(tert-butoxycarbonyl)-4-(phenoxycarbonylaminomethyl)piperidine and 1-methylhomopiperazine instead of pyrrolidine, the desired compound was obtained in 87% yield.

¹H NMR (300 MHz, CDCl₃) δ (TMS): 4.12 (m, 4H), 3.55 (m, 4H), 2.67 (broad t, J=12.3 Hz, 2H), 2.57 (m, 4H), 2.36 (s, 3H), 1.84 (m, 2H), 1.5-1.7 (m, 5H), 1.45 (s, 9H), 1.12 (m, 2H).

### b) Title Compound

Following a similar procedure to that described in section b of reference example 7, but using the compound obtained in the preceding section as starting product, the title compound of the example was obtained in 93% yield.

¹H NMR (300 MHz, CDCl₃) δ (TMS): 4.12 (t, J=6.3 Hz, 2H), 3.55 (m, 4H), 3.05 (m, 2H), 2.67 (m, 6H), 2.35 (s, 3H), 1.87 (m, 2H), 1.5-1.7 (m, 6H), 1.12 (m, 2H).

### REFERENCE EXAMPLE 62

### 4-[2-[(cis-2,6-Dimethylmorpholin-4-yl)carbonyloxy]ethyl]piperidine

### a) 1-(tert-Butoxycarbonyl)-4-[2-[(cis-2,6-dimethylmorpholin-4-yl)carbonyloxy]ethyl]piperidine

Following a similar procedure to that described in section b of reference example 37, but using 1-(tert-butoxycarbonyl)-4-[2-(phenoxycarbonyloxy)ethyl]piperidine (obtained in section b of reference example 25) instead of 1-(tert-butoxycarbonyl)4-(phenoxycarbonylaminomethyl)piperidine and cis-2,6-dimethylmorpholine instead of pyrrolidine, the desired compound was obtained in 86% yield.

¹H NMR (300 MHz, CDCl₃) δ (TMS): 3.84.2 (m, 6H), 3.53 (m, 2H), 2.68 (broad t, J=12.3 Hz, 2H), 2.51 (m, 2H), 1.5-1.7 (m, 5H), 1.45 (s, 9H), 1.17 (d, J=6 Hz, 6H), 1.08 (m, 2H).

### b) Title Compound

Following a similar procedure to that described in section b of reference example 7, but using the compound obtained in the preceding section as starting product, the title compound of the example was obtained in 88% yield.

¹H NMR (300 MHz, CDCl₃) δ (TMS): 4.13 (t, J=6.9 Hz, 2H), 3.91 (m, 2H), 3.53 (m, 2H), 3.05 (m, 2H), 2.58 (m, 4H), 1.7 (m, 2H), 1.61 (m, 2H), 1.48 (m, 2H), 1.17 (d, J=6 Hz, 6H), 1.08 (m, 2H).

### EXAMPLE 1

### Methyl (2S)-2-[1-(3,5-dichlorophenylsulfonyl)-L-prolylamino]-5-oxo-5-[4-(2-oxooxazolidin-3-ylmethyl)piperidin-1-yl]pentanoate

To a solution of 1-methyl N-[1-(3,5-dichlorophenylsulfonyl)-prolyl]-L-glutamate (obtained in reference example 48) (500 mg, 1.05 mmol) in DMF (15 mL), EDC.HCl (250 mg, 1.3 mmol), HOBT (170 mg, 1.3 mmol) and NMM (0.7 mL, 6.4 mmol) were added under argon atmosphere, and the resulting mixture was stirred at room temperature for 30 min. Next, 3-[(4-piperidyl)methyl]oxazolidin-2-one hydrochloride (obtained in reference example 6) (230 mg, 1.05 mmol) was added, and the mixture was further stirred overnight. The solvent was evaporated and the residue obtained was partitioned between CHCl₃ and saturated NaHCO₃ solution. The phases were separated, the organic phase was dried over sodium sulfate and concentrated to dryness, to afford a crude product which was purified by chromatography on silica gel using CHCl₃/MeOH mixtures of increasing polarity as eluent. 300 mg of the title compound of the example was obtained (45% yield).

¹H NMR (300 MHz, CDCl₃) δ (TMS): 7.76 (s, 2H), 7.66 (m, 1H), 7.59 (s, 1H), 4.61 (m, 1H), 4.49 (m, 1H), 4.32 (m, 2H), 4.13 (m, 1H), 3.80 (m, 1H), 3.76 (s, 3H), 3.57 (m, 3H), 3.25 (m, 1H), 3.14 (m, 2H), 3.00 (m, 1H), 2.66 (m, 1H), 2.43 (m, 2H), 2.22 (m, 1H), 2.16 (m, 2H), 1.59-1.93 (complex signal, 6H), 1.20 (m, 2H).

Following a similar procedure to that described in example 1, but starting in each case from the corresponding acid and amine, the following compounds were obtained:

### EXAMPLE 2

### Methyl (2S)-2-[1-(3,5-dichlorophenylsulfonyl)-L-prolylamino]-5-[4-(2-oxopyrrolidin-1-ylmethyl)piperidin-1-yl]-5-oxopentanoate

Starting compounds: reference examples 48 and 3; 86% yield.

¹H NMR (300 MHz, CDCl₃) δ (TMS): 7.76 (s, 2H), 7.69 (d, J=7.0 Hz, 1H), 7.59 (s, 1H), 4.60 (m, 1H), 4.47 (m, 1H), 4.11 (m, 1H), 3.81 (m, 1H), 3.75 (s, 3H), 3.58 (m, 1H), 3.37 (m, 2H), 3.21 (m, 2H), 3.08 (m, 1H), 2.99 (m, 1H), 2.32-2.65 (complex signal, 5H), 1.98-2.30 (complex signal, 5H), 1.60-1.90 (complex signal, 6H), 1.19 (m, 2H).

### EXAMPLE 3

### Methyl (2S)-2-[1-(3,5-dichlorophenylsulfonyl)-L-prolylamino]-5-oxo-5-[4-(1-oxoisoindolin-2-ylmethyl)piperidin-1-yl]pentanoate

Starting compounds: reference examples 48 and 4; 97% yield.

¹H NMR (300 MHz, CDCl₃) δ (TMS): 7.82 (d, J=7.2 Hz, 1H), 7.75 (s, 2H), 7.68 (m, 1H), 7.57 (m, 1H), 7.51 (m, 3H), 4.53 (m, 1H), 4.46 (m, 1H), 4.39 (s, 2H), 4.12 (m, 1H), 3.79 (m, 1H), 3.74 (s, 3H), 3.54 (m, 2H), 3.46 (m, 1H), 3.23 (m, 1H), 2.97 (m, 1H), 2.60 (m, 1H), 2.41 (m, 3H), 1.69-2.30 (complex signal, 8H), 1.24 (m, 2H).

### EXAMPLE 4

### Methyl (2S#-2-[1-(3,5-dichlorophenylsulfonyl)-L-prolylamino]-5-[4-(2-ethyl-5,7-dimethylimidazo[4,5-b]pyridin-3-ylmethyl)piperidin-1-yl]-5-oxopentanoate

Starting compounds: reference examples 48 and 9; 65% yield.

¹H NMR (300 MHz, CDCl₃) δ (TMS): 7.76 (s, 2H), 7.69 (d, J=6 Hz, 1H), 7.59 (s, 1H), 6.84 (s, 1H), 4.63 (m, 1H), 4.47 (m, 1H), 4.07 (m, 3H), 3.81 (m, 1H), 3.74 (s, 3H), 3.48 (m, 1H), 3.22 (m, 1H), 2.86 (m, 3H), 2.59 (s, 3H), 2.55 (s, 3H), 2.46 (m, 3H), 2.05-2.35 (complex signal, 4H), 1.95 (m, 3H), 1.62 (m, 2H), 1.40 (q, J=7.4 Hz, 3H), 1.25(m, 2H).

### EXAMPLE 5

### Methyl (2S)-5-[4-(2-methylimidazo[4,5-c]pyridin-1-ylmethyl)piperidin-1-yl]-5-oxo-2[1 tosyl-L-prolylamino]pentanoate

Starting compounds: reference examples 49 and 8; 47% yield.

¹H NMR (300 MHz, CDCl₃) δ (TMS): 8.86 (s, 1H), 8.21 (d, J=5.5 Hz, 1H), 7.66 (d, J=7.8 Hz, 2H), 7.57 (d, J=7.3 Hz, 1H), 7.28 (d, J=7.8 Hz, 2H), 7.19 (d, J=5.5 Hz, 1H), 4.50 (m, 2H), 4.01 (m, 1H), 3.95 (d, J=7.7 Hz, 2H), 3.82 (m, 1H), 3.68 (s, 3H), 3.44 (m, 1H), 3.15 (m, 1H), 2.60-2.90 (complex signal, 3H), 2.57 (s, 3H), 2.42 (m, 1H), 2.36 (s, 3H), 2.12 (m, 1H), 2.03 (m, 3H), 1.77 (m, 1H), 1.50 (m, 4H), 1.25 (m, 2H).

### EXAMPLE 6

### Methyl (2S)-5-oxo-5-[4-[(2-oxopyrrolidin-1-yl)methyl]piperidin-1-yl]-2-[1-tosyl-L-prolylamino]pentanoate

Starting compounds: reference examples 49 and 3; 37% yield.

¹H NMR (300 MHz, CDCl₃) δ (TMS): 7.73 (d, J=8.2 Hz, 2H), 7.58 (t, J=7.3 Hz, 1H), 7.33 (d, J=8.2 Hz, 2H), 4.54 (m, 2H), 4.05 (m, 1H), 3.82 (m, 1H), 3.75 (s, 3H), 3.53 (m, 1H), 3.37 (m, 2H), 3.17 (m, 2H), 3.09 (m, 1H), 2.98 (m, 1H), 2.59 (m, 1H), 2.43 (s, 3H), 2.36 (m, 3H), 2.28 (m, 1H), 2-2.2 (complex signal, 4H), 1.85(m, 3H), 1.63(m, 4H), 1.18(m, 2H).

### EXAMPLE 7

### Methyl (2S)-5-oxo-5-[4-(2-phenylimidazol-1-ylmethyl)piperidin-1-yl-2-[1-tosyl-L-prolylamino]pentanoate

Starting compounds: reference examples 49 and 7; 42% yield.

¹H NMR (300 MHz, CDCl₃) δ (TMS): 7.73 (d, J=8.0 Hz, 2H), 7.52 (m, 2H), 7.50 (m, 1H), 7.41 (m, 3H), 7.34 (d, J=8.0 Hz, 2H), 7.11 (m, 1H), 6.96 (m, 1H), 4.53 (m, 2H), 4.05 (m, 1H), 3.89 (d, J=7.3 Hz, 2H), 3.82 (m, 1H), 3.74 (s, 3H), 3.47 (m, 1H), 3.14 (m, 1H), 2.92 (m, 1H), 2.44 (s, 3H), 2.42 (m, 3H), 2.27 (m, 1H), 2.09 (m, 2H), 1.50-1.85 (complex signal, 6H), 1.15 (m, 2H).

### EXAMPLE 8

### Methyl (2S)-5-[4-[[1-(2-ethoxyethyl)benzimidazol-2-yl]methyl]piperazin-1-yl]-5-oxo-2-[[1-tosyl-L-prolyl]amino]pentanoate

Starting compounds: reference examples 49 and 10; 50% yield.

¹H NMR (300 MHz, CDCl₃) δ (TMS): 7.73 (d, J=8.2 Hz, 2H), 7.71 (m, 1H), 7.54 (d, J=7.5 Hz, 1H), 7.33 (d, J=8.2 Hz, 2H), 7.31 (m, 1H), 7.23 (m, 2H), 4.50 (m, 3H), 4.07 (m, 1H), 3.88 (s, 2H), 3.76 (s, 3H), 3.74 (m, 2H), 3.61 (m, 2H), 3.56 (m, 1H), 3.44 (m, 2H), 3.37 (q, J=7 Hz, 2H), 3.15 (m, 1H), 2.51 (m, 4H), 2.47 (m, 2H), 2.45 (s, 3H), 2.30 (m, 1H), 2.12 (m, 2H), 1.85 (m, 1H), 1.63 (m, 2H), 1.10(t, J=7 Hz, 3H).

### EXAMPLE 9

### Methyl (2S)-5-oxo-5-[4-(2-pyridylmethyl)piperazin-1-yl]-2-[[1-tosyl-L-prolyl]amino]pentanoate

Starting compounds: reference examples 49 and 11; 58% yield.

¹H NMR (300 MHz, CDCl₃) δ (TMS): 8.57 (m, 1H), 7.74 (d, J=8.2 Hz, 2H), 7.65 (m, 1H), 7.55 (d, J=7.5 Hz, 1H), 7.39 (m, 1H), 7.35 (d, J=8.2 Hz, 2H), 7.17 (m, 1H), 4.53 (m, 1H), 4.08 (m, 1H), 3.76 (s, 3H), 3.66 (m, 2H), 3.63 (s, 2H), 3.53 (m, 1H), 3.48 (m, 2H), 3.18 (m, 1H), 2.49 (m, 4H), 2.46 (m, 2H), 2.44 (s, 3H), 2.25 (m, 1H), 2.20 (m, 1H), 2.10 (m, 2H), 1.92 (m, 1H), 1.65 (m, 1H).

### EXAMPLE 10

### Methyl (2S)-5-oxo-5-[4-(1-oxoisoindolin-2-ylmethyl)piperidin-1-yl]-2-[[1-tosyl-L-prolyl]amino]pentanoate

Starting compounds: reference examples 49 and 4; 56% yield.

¹H NMR (300 MHz, CDCl₃) δ (TMS): 7.83 (d, J=7.9 Hz, 1H), 7.74 (d, J=8.2 Hz, 2H), 7.51 (m, 4H), 7.34 (d, J=8.2 Hz, 2H), 4.54 (m, 2H), 4.39 (m, 2H), 4.07 (m, 1H), 3.82 (m, 1H), 3.76 (s, 3H), 3.53 (m, 2H), 3.46 (m, 1H), 3.16 (m, 1H), 3.01 (m, 1H), 2.63 (m, 1H), 2.44 (s, 3H), 2.39 (m, 3H), 2.13 (m, 3H), 1.65-1.95 (complex signal, 5H), 1.25 (m, 2H).

### EXAMPLE 11

### Methyl (2S)-5-oxo-5-[4-(2-thienylmethyl)piperazin-1-yl]-2-[[1-tosyl-L-prolyl]amino]pentanoate

Starting compounds: reference examples 49 and 12; 38% yield.

¹H NMR (300 MHz, CDCl₃) δ (TMS): 7.73 (d, J=8.2 Hz, 2H), 7.54 (d, J=7.5 Hz, 1H), 7.34 (d, J=8.2 Hz, 2H), 7.22 (m, 1H), 6.93 (m, 2H), 4.54 (m, 1H), 4.07 (m, 1H), 3.76 (s, 3H), 3.71 (s, 2H), 3.61 (m, 2H), 3.52 (m, 1H), 3.46 (m, 2H), 3.17 (m, 1H), 2.40 (m, 5H), 2.29 (m, 1H), 2.44 (s, 3H), 2.13 (m, 2H), 1.83 (m, 1H), 1.62 (m, 3H).

### EXAMPLE 12

### Methyl (2S)-5-[4-[(2,5-dioxopyrrolidin-1-yl)methyl]piperidin-1-yl]-5-oxo-2-[[1-tosyl-L-prolyl]amino]pentanoate

Starting compounds: reference examples 49 and 5; 33% yield.

¹H NMR (300 MHz, CDCl₃) δ (TMS): 7.75 (d, J=8.2 Hz, 2H), 7.58 (m, 1H), 7.34 (d, J=8.2 Hz, 2H), 4.54 (m, 2H), 4.07 (d, J=7.9 Hz, 1H), 3.84 (m, 1H), 3.76 (s, 3H), 3.54 (m, 1H), 3.41 (m, 2H), 3.17 (m, 1H), 2.93 (m, 1H), 2.71 (m, 4H), 2.54 (m, 1H), 2.44 (s, 3H), 2.42 (m, 1H), 2.32 (m, 1H), 2.17 (m, 3H), 2.10 (m, 2H), 1.6-1.9 (complex signal, 4H), 1.16 (m, 2H).

### EXAMPLE 13

### Methyl (2S)-2-[1-(3,5-dichlorophenylsulfonyl)-L-prolylamino]-5-[4-[(3-methylbutanoylamino)methyl]piperidin-1-yl]-5-oxopentanoate

Starting compounds: reference examples 48 and 14; 68% yield.

¹H NMR (300 MHz, CDCl₃) δ (TMS): 7.76 (s, 2H), 7.70 (m, 1H), 7.60 (s, 1H), 5.55 (m, 1H), 4.65 (m, 1H), 4.51 (m, 1H), 4.12 (m, 1H), 3.82 (m, 1H), 3.76 (s, 3H), 3.62 (m, 1H), 3.18 (m, 3H), 2.40 (m, 3H), 1.7-2.30 (m, 10H), 1.58 (m, 3H), 1.15 (m, 2H), 0.93 (d, J=6.2 Hz, 6H).

### EXAMPLE 14

### Methyl (2S)-5-[4-[(N′-tert-butylureido)methyl]piperidin-1-yl]-2-[1-(3,5-dichlorophenylsulfonyl)-L-prolylamino]-5-oxopentanoate

Starting compounds: reference examples 48 and 15; 48% yield.

¹H NMR (300 MHz, CDCl₃) δ (TMS): 7.77 (s, 2H), 7.72 (m, 1H), 7.61 (s, 1H), 4.65 (m, 1H), 4.55 (m, 1H), 4.50 (m, 1H), 4.13 (m, 2H), 3.81 (m, 1H), 3.76 (s, 3H), 3.65 (m, 1H), 3.18 (m, 4H), 1.55-2.60 (m, 12H), 1.31 (s, 9H), 1.20 (m, 2H).

### EXAMPLE 15

### Methyl (2S)-2-[1-(3,5-dichlorophenylsulfonyl)-L-prolylamino]-5-[4-[(isobutoxycarbonylamino)methyl]piperidin-1-yl]-5-oxopentanoate

Starting compounds: reference examples 48 and 16; 24% yield.

¹H NMR (300 MHz, CDCl₃) δ (TMS): 7.77 (s, 2H), 7.71 (m, 1H), 7.59 (s, 1H), 4.75 (m, 1H), 4.62 (m, 1H), 4.49 (m, 1H), 4.12 (m, 1H), 3.82 (m, 3H), 3.76 (s, 3H), 3.59 (m, 1H), 3.26 (m, 1H), 3.02 (m, 3H), 2.46 (m, 3H), 2.14 (m, 3H), 1.65-1.95 (broad m, 7H), 1.16 (m, 2H), 0.91 (d, J=6.7 Hz, 6H).

### EXAMPLE 16

### Methyl (2S)-5-oxo-2-[1-tosyl-L-prolyl]amino-5-[4-[[4-(trifluoromethyl)pyrimidin-2-yl]aminomethyl]piperidin-1-yl]pentanoate

Starting compounds: reference examples 49 and 26; 33% yield.

¹H NMR (300 MHz, CDCl₃) δ (TMS): 8.43 (m, 1H), 7.73 (d, J=8.0 Hz, 2H), 7.56 (m, 1H), 7.32 (d, J=8.0 Hz, 2H), 6.79 (m, 1H), 5.64 (m, 1H), 4.52 (m, 2H), 4.06 (m, 1H), 3.82 (m, 1H), 3.74 (s, 3H), 3.51 (m, 1H), 3.35 (m, 2H), 3.15 (m, 1H), 2.98 (m, 1H), 2.42 (s, 3H), 1.9-2.6 (complex signal, 6H), 1.76 (m, 4H), 1.58 (m, 2H), 1.21 (m, 2H).

### EXAMPLE 17

### Methyl (2S)-2-[1-(3,5-dichlorophenylsulfonyl)-L-prolylamino]-5-[4-[(isopropylsulfonylamino)methyl]piperidin-1-yl]-5-oxopentanoate

### a) Methyl (2S)-2-[1-(3,5-dichlorophenylsulfonyl)-L-prolylamino]-5-(2,5-dioxopyrrolidinyloxy)-5-oxopentanoate

To a solution of, 1-methyl N-[1-(3,5-dichlorophenylsulfonyl)-L-prolyl]-L-glutamate (obtained in reference example 48) (0.56 g, 1.2 mmol), and NHS (0.14 g, 1.2 mmol) in CHCl₃ (25 mL), cooled to 0° C. and under argon atmosphere, DCC (0.25 g, 1.2 mmol) was added, and the resulting mixture was stirred overnight at room temperature. The solid obtained was filtered off and the solution was concentrated to dryness. A crude product was obtained, which was directly used in the following step.

### b) Title Compound

In this second step, methyl (2S)-2-[1-(3,5-dichlorophenylsulfonyl)-L-prolylamino]-5-(2,5-dioxopyrrolidinyloxy)-5-oxopentanoate (obtained in the preceding section) (0.67 g, 1.2 mmol) was dissolved in CHCl₃ (25 mL). TEA (0.7 mL, 5 mmol) was added, and then the amine obtained in reference example 17 (0.31 g, 1.2 mmol) was added slowly and portionwise. The solution was stirred overnight at room temperature. The product was partitioned between CHCl₃ and 0.2 M NaHCO₃. The crude product obtained was purified by chromatography on silica gel using a CHCl₃:MeOH 2% mixture as eluent. 0.78 g of the title compound of the example was obtained (97% yield).

¹H NMR (300 MHz, CDCl₃) δ (TMS): 7.77 (s, 2H), 7.70 (m, 1H), 7.59 (s, 1H), 4.68 (m, 1H), 4.51 (m, 1H), 4.22 (m, 0.5H), 4.10 (m, 1.5H), 3.85 (m, 1H), 3.76 (s, 3H), 3.62 (m, 1H), 3.18 (m, 2H), 3.01 (m, 3H), 2.48 (m, 3H), 2.13 (m, 3H), 1.92 (m, 1H), 1.76 (m, 5H), 1.35 (d, J=6.8 Hz, 6H), 1.16 (m, 2H).

Following a similar procedure to that described in example 17, but using in each case a suitable acid and amine, in the first and second step, respectively, the following compounds were obtained:

### EXAMPLE 18

### Methyl (2S)-2-[1-(3,5-dichlorophenylsulfonyl)-L-prolylamino]-5-[4-[(N′-isopropylthioureido)methyl]piperidin-1-yl]-5-oxopentanoate

Compounds used: reference examples 48 and 18; 55% yield.

¹H NMR (300 MHz, CDCl₃) δ (TMS): 7.83 (s, 2H), 7.76 (m, 1H), 7.68 (s, 1H), 5.85 (broad s, 0.5H), 5.75 (broad s, 1.5H), 4.74 (m, 1H), 4.62 (m, 0.5H), 4.50 (m, 0.5H), 4.20 (m, 2H), 3.95 (m, 1H), 3.85 (s, 1.5H), 3.83 (s, 1.5H), 3.72 (m, 0.5H), 3.62 (m, 0.5H), 3.51 (m, 2H), 3.31 (m, 1H), 3.08 (m, 1H), 2.55 (m, 3H), 2.28 (m, 2H), 2.05 (m, 3H), 1.88 (m, 4H), 1.30 (m, 8H).

### EXAMPLE 19

### Methyl (2S)-2-[1-(3,5-dichlorophenylsulfonyl)-L-prolylamino]-5-[4-12-(2-ethyl-5,7-dimethylimidazo[4,5-b]pyridin-3-yl)ethyl]piperidin-1-yl]-5-oxopentanoate

Compounds used: reference examples 48 and 28; 57% yield.

¹H NMR (300 MHz, CDCl₃) δ (TMS): 7.77 (s, 2H), 7.71 (m, 1H), 7.59 (s, 1H), 6.83 (s, 1H), 4.59 (m, 1H), 4.48 (m, 1H), 4.23 (m, 2H), 4.12 (m, 1H), 3.80 (m, 1H), 3.76 (s, 3H), 3.60 (m, 1H), 3.22 (m, 1H), 2.91 (m, 3H), 2.59 (s, 3H), 2.53 (s, 3H), 2.45 (m, 3H), 2.14 (m, 3H), 1.7-1.95 (m, 8H), 1.42 (t, J=6.8 Hz, 3H), 1.25 (m, 2H).

### EXAMPLE 20

### Methyl (2S)-2-[1-(3,5-dichlorophenylsulfonyl)-L-prolylamino]-5-oxo-5-[4-[2-(pyrrolidin-1-ylcarbonyloxy)ethyl]piperidin-1-yl]pentanoate

Compounds used: reference examples 48 and 25; 76% yield.

¹H NMR (300 MHz, CDCl₃) δ (TMS): 7.77 (s, 2H), 7.71 (m, 1H), 7.59 (s, 1H), 4.57 (m, 1H), 4.48 (m, 1H), 4.10 (m, 3H), 3.80 (m, 1H), 3.75 (s, 3H), 3.59 (m, 1H), 3.30 (m, 5H), 2.98 (m, 1H), 2.47 (m, 3H), 2.15 (m, 3H), 1.85 (m, 9H), 1.72 (m, 3H), 1.13 (m, 2H).

### EXAMPLE 21

### Methyl (2S)-2-[1-(3,5-dichlorophenylsulfonyl)-L-prolylamino]-5-[4-(4-methylpiperazin-1-ylmethyl)piperidin-1-yl]-5-oxopentanoate

Compounds used: reference examples 48 and 27; 76% yield.

¹H NMR (300 MHz, CDCl₃) δ (TMS): 7.77 (s, 2H), 7.73 (m, 1H), 7.71 (s, 1H), 4.61 (m, 1H), 4.48 (m, 1H), 4.14 (m, 1H), 3.82 (m, 1H), 3.75 (s, 3H), 3.58 (m, 1H), 3.24 (m, 1H), 2.97 (m, 1H), ?0.42 (m, 1H), 2.27(s, 3H), 2.16 (m, 5H), 1.82 (m, 6H), 1.09 (m, 2H).

### EXAMPLE 22

### Methyl (2S)-2-[1-(3,5-dichlorophenylsulfonyl)-L-prolylamino]-5-[4-[2-(4-morpholinyl)ethyl]piperidin-1-yl]-5-oxopentanoate

Compounds used: reference examples 48 and 29; 88% yield.

¹H NMR (300 MHz, CDCl₃) δ (TMS): 7.83 (s, 2H), 7.78 (m, 1H), 7.66 (s, 1H), 4.64 (m, 1H), 4.52 (m, 1H), 4.19 (m, 1H), 3.87 (m, 1H), 3.76 (s, 3H), 3.67 (m, 4H), 3.62 (m, 1H), 3.30 (m, 1H), 3.03 (m, 1H), 2.50 (m, 8H), 2.26 (m, 3H), 1.8-2 (m, 6H), 1.55 (m, 2H), 1.20 (m, 3H).

### EXAMPLE 23

### Methyl (2S)-2-[1-(3,5-dichlorophenylsulfonyl)-L-prolylamino]-5-[4-[[(dimethylaminoacetyl)amino]methyl]piperidin-1-yl]-5-oxopentanoate

Compounds used: reference examples 48 and 30; 10% yield.

¹H NMR (300 MHz, CDCl₃) δ (TMS): 7.76 (s, 2H), 7.70 (m, 1H), 7.60 (s, 1H), 7.32 (m, 1H), 4.66 (m, 1H), 4.48 (m, 1H), 4.13 (m, 1H), 3.80 (m, 1H), 3.76 (s, 3H), 3.60 (m, 1H), 3.20 (m, 3H), 2.97 (m, 1H), 2.95 (s, 2H), 2.55 (m, 1H), 2.43 (m, 2H), 2.29 (s, 6H), 2.20 (m, 2H), 1.88 (m, 5H), 1.60 (m, 2H), 1.16 (m, 2H).

### EXAMPLE 24

### Methyl (2S)-2-[1-(3,5-dichlorophenylsulfonyl)-L-prolylamino]-5-[4-[2-(diethylamino)ethyl]piperidin-1-yl]-5-oxopentanoate

Compounds used: reference examples 48 and 33; 59% yield.

¹H NMR (300 MHz, CDCl₃) δ (TMS): 7.76 (s, 2H), 7.72 (d, J=7.1 Hz, 1H), 7.59 (s, 1H), 4.55 (m, 1H), 4.48 (m, 1H), 4.14 (m, 1H), 3.80 (m, 1H), 3.75 (s, 3H), 3.58 (m, 1H), 3.25 (m, 1H), 2.97 (m, 1H), 2.49 (m, 10H), 2.15 (m, 3H), 1.74 (m, 4H), 1.41 (m, 3H), 1.10 (m, 2H), 0.99 (m, 6H).

### EXAMPLE 25

### Methyl (2S)-2-[1-(3,5-dichlorophenylsulfonyl)-L-prolylamino]-5-oxo-5-[4-[2-(1-pyrrolidinyl)ethyl]piperidin-1-yl]pentanoate

Compounds used: reference examples 48 and 34; 79% yield.

¹H NMR (300 MHz, CDCl₃) δ (TMS): 7.77 (s, 2H), 7.74 (d, J=7.1 Hz, 1H), 7.58 (s, 1H), 4.55 (m, 1H), 4.48 (m, 1H), 4.14 (m, 1H), 3.79 (m, 1H), 3.75 (s, 3H), 3.58 (m, 1H), 3.24 (s, 1H), 2.97 (m, 1H), 2.48 (m, 9H), 2.16 (m, 4H), 1.82 (m, 8H), 1.44 (m, 3H), 1.10 (m, 2H).

### EXAMPLE 26

### Methyl (2S)-2-[1-(3,5-dichlorophenylsulfonyl)-L-prolylamino]-5-[4-[2-[(4-methylpiperazin-1-yl)carbonyloxy]ethyl]piperidin-1-yl]-5-oxopentanoate

Compounds used: reference examples 48 and 35.

Obtained as a crude product.

### EXAMPLE 27

### Methyl (2S)-2-[1-(3,5-dichlorophenylsulfonyl)-L-prolylamino]-5-[4-[2-[(2-methoxyethyl)aminocarbonyloxy]ethyl]piperidin-1-yl]-5-oxopentanoate

Compounds used: reference examples 48 and 36; 47% yield.

¹H NMR (300 MHz, CDCl₃) δ (TMS): 7.77 (s, 2H), 7.71 (m, 1H), 7.59 (s, 1H), 4.99 (m, 1H), 4.58 (m, 1H), 4.40 (m, 1H), 4.10 (m, 3H), 3.79 (m, 1H), 3.75 (s, 3H), 3.60 (m, 1H), 3.47 (m, 2H), 3.38 (s, 3H), 3.18 (s, 1H), 2.98 (m, 1H), 2.48 (m, 3H), 2.16 (m, 4H), 1.90 (m, 1H), 1.75 (m, 6H), 1.54 (m, 2H), 1.15 (m, 2H).

### EXAMPLE 28

### Methyl (2S)-2-[1-(3,5-dichlorophenylsulfonyl)-L-prolylamino]-5-[4-(4-morpholinylcarbonylaminomethyl)piperidin-1-yl]-5-oxopentanoate

Compounds used: reference examples 48 and 38; 72% yield.

¹H NMR (300 MHz, CDCl₃) δ (TMS): 7.76 (s, 2H), 7.68 (m, 1H), 7.60 (s, 1H), 4.62 (m, 2H), 4.49 (m, 1H), 4.11 (m, 1H), 3.80 (m, 1H), 3.78 (s, 3H), 3.68 (m, 4H), 3.66 (m, 1H), 3.34 (m, 4H), 3.23 (m, 3H), 3.00 (m, 1H), 2.44 (m, 3H), 2.15 (m, 3H), 1.80 (m, 6H), 1.16 (m, 2H).

### EXAMPLE 29

### Methyl (2S)-2-[1-(3,5-dichlorophenylsulfonyl)-L-prolylamino]-5-oxo-5-[4-(1-pyrrolidinylcarbonylaminomethyl)piperidin-1-yl]pentanoate

Compounds used: reference examples 48 and 37; 78% yield.

¹H NMR (300 MHz, CDCl₃) δ (TMS): 7.77 (s, 2H), 7.71 (m, 1H), 7.60 (s, 1H), 4.63 (m, 1H), 4.48 (m, 1H), 4.29 (m, 1H), 4.13 (m, 1H), 3.80 (m, 1H), 3.76 (s, 3H), 3.61 (m, 1H), 3.31 (m, 4H), 3.29 (m, 1H), 3.14 (m, 2H), 2.99 (m, 1H), 2.47 (m, 2H), 2.15 (m, 2H), 1.89 (m, 6H), 1.78 (m, 4H), 1.52 (m, 2H), 1.12 (m, 2H).

### EXAMPLE 30

### Methyl (2S)-2-[1-(3,5-dichlorophenylsulfonyl)-L-prolylamino]-5-oxo-5-[4-(1-piperidylmethyl)piperidin-1-yl]pentanoate

Compounds used: reference examples 48 and 39; 65% yield.

¹H NMR (300 MHz, CDCl₃) δ (TMS): 7.78 (s, 2H), 7.72 (m, 1H), 7.59 (s, 1H), 4.56 (m, 1H), 4.46 (m, 1H), 4.14 (m, 1H), 3.81 (m, 1H), 3.75 (s, 3H), 3.58 (m, 1H), 3.24 (m, 1H), 2.97 (m, 1H), 2.48 (m, 3H), 2.20 (m, 4H), 2.11 (m, 4H), 1.82 (m, 7H), 1.56 (m, 4H), 1.42 (m, 2H), 1.05 (m, 2H).

### EXAMPLE 31

### Methyl (2S)-2-[1-(3,5-dichlorophenylsulfonyl)-L-prolylamino]-5-[4-[[N-ethyl-N-(trifluoroacetyl)amino]methyl]piperidin-1-yl]-5-oxopentanoate

Compounds used: reference examples 48 and 40; 67% yield.

¹H NMR (300 MHz, CDCl₃) δ (TMS): 7.76 (s, 2H), 7.68 (m, 1H), 7.59 (s, 1H), 4.70 (m, 1H), 4.48 (m, 1H), 4.12 (m, 1H), 3.80 (m, 1H), 3.75 (s, 3H), 3.56 (m, 1H), 3.44 (m, 2H), 3.31 (m, 1H), 3.23 (m, 2H), 2.95 (m, 1H) 2.48 (m, 3H), 2.15 (m, 3H), 1.89 (m, 4H), 1.66 (m, 2H), 1.16 (m, 5H).

### EXAMPLE 32

### Methyl (2S)-2-[1-(3,5-dichlorophenylsulfonyl)-L-prolylamino]-5-[4-[[(4-methylpiperazin-1-yl)carbonylamino]methyl]piperidin-1-yl]-5-oxopentanoate

Compounds used: reference examples 48 and 41.

Obtained as a crude product.

### EXAMPLE 33

### Methyl (2S)-2-[1-(3,5-dichlorophenylsulfonyl)-L-prolylamino]-5-oxo-5-[444-pyridylaminomethyl)piperidin-1-yl]pentanoate

Compounds used: reference examples 48 and 42; 52% yield.

¹H NMR (300 MHz, CDCl₃) δ (TMS): 8.17 (d, J=5.8 Hz, 2H), 7.76 (s, 2H), 7.66 (m, 1H), 7.60 (m, 1H), 6.42 (d, J=5.8 Hz, 2H), 4.65 (m, 1H), 4.45 (m, 1H), 4.30 (m, 0.5H), 4.25 (m, 0.5H), 4.08 (m, 1H), 3.87 (m, 1H), 3.76 (s, 3H), 3.57 (m, 1H), 3.20 (m, 1H), 3.05 (m, 3H), 2.48 (m, 3H), 2.20 (m, 3H), 1.80 (m, 6H), 1.22 (m, 2H).

### EXAMPLE 34

### Methyl (2S)-2-[1-(3,5-dichlorophenylsulfonyl)-L-prolylamino]-5-[4-[(N-ethyl-N-isobutoxycarbonylamino)methyl]piperidin-1-yl]-5-oxopentanoate

Compounds used: reference examples 48 and 43; 29% yield.

¹H NMR (300 MHz, CDCl₃) δ (TMS): 7.77 (s, 2H), 7.72 (m, 1H), 7.59 (s, 1H), 4.61 (m, 1H), 4.49 (m, 1H), 4.13 (m, 1H), 4.00 (m, 1H), 3.83 (m, 2H), 3.79 (s, 3H), 3.59 (m, 1H), 3.23 (m, 2H), 3.08 (m, 1H), 2.99 (m, 1H), 2.46 (m, 2H), 2.18 (m, 2H), 1.92 (m, 4H), 1.70 (m, 3H), 1.35 (m, 2H), 1.15 (m, 5H), 0.93 (d, J=6.5 Hz, 6H).

Following a similar procedure to that described in section a of reference example 51, but starting in each case from a suitable acid and amine, and adding TEA to the reaction medium the following compounds were obtained:

### EXAMPLE 35

### Methyl (2S)-2-[1-(3,5-dichlorophenylsulfonyl)-L-prolylamino]-5-[4-(2-methylimidazo[4,5-c]pyridin-1-ylmethyl)piperidin-1-yl]-5-oxopentanoate

Starting compounds: reference examples 47 and 51; 50% yield.

¹H NMR (300 MHz, CDCl₃) δ (TMS): 8.96 (s; 1H), 8.33 (m, 1H), 7.78 (s, 2H 50%), 7.75 (s, 2H 50%), 7.61 (s, 1H), 7.58 (m, 1H), 7.20 (m, 1H), 4.71 (m, 1H), 4.54 (m, 1H), 4.11 (m, 1H), 3.98 (m, 2H), 3.81 (m, 1H), 3.75 (s, 3H 50%), 3.74 (s, 3H 50%), 3.52 (m, 1H), 3.22 (m, 1H), 2.95 (m, 1H), 2.62 (s, 3H 50%), 2.58 (s, 3H 50%), 2.44 (m, 2H), 1.60-2.30 (complex signal, 6H), 1.55 (m, 4H), 1.25 (m, 2H).

### EXAMPLE 36

### Methyl (2S)-2-[1-(3,5-dichlorophenylsulfonyl)-L-prolylamino]-5-oxo-5-[4-(1-pyrrolylmethyl)piperidin-1-yl]pentanoate

Starting compounds: reference examples 48 and 19; 29% yield.

¹H NMR (300 MHz, CDCl₃) δ (TMS): 7.76 (s, 2H), 7.67 (m, 1H), 7.60 (s, 1H), 6.59 (m, 2H), 6.13 (m, 2H), 4.66 (m, 1H), 4.48 (m, 1H), 4.12 (m, 1H), 4.00 (m, 1H), 3.75 (s, 3H), 3.71 (m, 2H), 3.55 (m, 1H), 3.48 (m, 1H), 3.23 (m, 1H), 2.98 (m, 1H), 2.45 (m, 2H), 2.22 (m, 1H), 2.18 (m, 2H), 1.99 (m, 3H), 1.57 (m, 3H), 1.20 (m, 2H).

### EXAMPLE 37

### Methyl (2S)-2-1-(3,5-dichlorophenylsulfonyl)-L-prolylamino]-5-[4-(2,5-dimethylpyrrol-1-ylmethyl)piperidin-1-yl]-5-oxopentanoate

Starting compounds: reference examples 48 and 20.

Obtained as a crude product.

### EXAMPLE 38

### Methyl (2S)-2-[1-(3,5-dichlorophenylsulfonyl)-L-prolylamino]-5-[4-dimethylaminomethyl)piperidin-1-yl]-5-oxopentanoate

Starting compounds: reference examples 48 and 21; 54% yield.

¹H NMR (300 MHz, CDCl₃) δ (TMS): 7.78 (s, 2H), 7.72 (d, J=7.2 Hz, 1H), 7.59 (s, 1H), 4.58 (m, 1H), 4.48 (m, 1H), 4.15 (m, 1H), 3.78 (m, 1H), 3.75 (s, 3H), 3.58 (m, 1H), 3.26 (m, 1H), 2.98 (m, 1H), 2.55 (m, 1H), 2.45 (m, 2H), 2.16 (s, 6H), 2.12 (m, 4H), 1.92 (m, 3H), 1.73 (m, 2H), 1.65 (m, 2H), 1.10 (m, 2H).

### EXAMPLE 39

### Methyl (2S)-2-[1-(3,5-dichlorophenylsulfonyl)-L-prolylamino]-5-[4-[2-(dimethylamino)ethyl]piperazin-1-yl]-5-oxopentanoate

Starting compounds: reference examples 48 and 22; 38% yield.

¹H NMR (300 MHz, CDCl₃) δ (TMS): 7.77 (s, 2H), 7.64 (d, J=7.2 Hz, 1H), 7.59 (s, 1H), 4.49 (m, 1H), 4.15 (m, 1H), 3.76 (s, 3H), 3.60 (m, 3H), 3.41 (m, 2H), 3.26 (m, 1H), 2.47 (m, 10H), 2.30 (s, 6H), 2.15 (m, 1H), 2.05 (m, 2H), 1.95(m, 2H), 1.80 (m, 1H).

### EXAMPLE 40

### Methyl (2S)-2-[1-(3,5-dichlorophenylsulfonyl)-L-prolylamino]-5-[4-(2-ethylimidazo[4,5-c]pyridin-1-ylmethyl)piperidin-1-yl]-5-oxopentanoate

Starting compounds: reference examples 48 and 23; 62% yield.

¹H NMR (300 MHz, CDCl₃) δ (TMS): 8.96 (s, 1H), 8.28 (m, 1H), 7.73 (s, 1H), 7.71 (s, 1H), 7.67 (d, J=7.1 Hz, 1H), 7.56 (s, 1H), 7.19 (m, 1H), 4.66 (broad d, J=13 Hz, 1H), 4.50 (m, 1H), 4.08 (m, 1H), 3.94 (m, 2H), 3.80 (broad d, J=13 Hz, 1H), 3.72 (s, 1.5H), 3.70 (s, 1.5H), 3.56 (m, 1H), 3.40 (m, 1H), 3.15 (m, 1H), 2.86 (m, 2H), 2.40 (m, 3H), 2.25 (m, 1H), 2.00 (m, 4H), 1.90 (m, 2H), 1.57 (m, 2H), 1.42 (q, J=7.1 Hz, 3H), 1.27 (m, 2H).

### EXAMPLE 41

### Methyl (2S)-2-[1-(3,5-dichlorophenylsulfonyl)-L-prolylamino]-5-oxo-5-[4-(2-oxo-2,3-dihydroimidazo[4,5-c]pyridin-1-ylmethyl)piperidin-1-yl]pentanoate

Starting compounds: reference examples 48 and 24; 33% yield.

¹H NMR (300 MHz, CDCl₃) δ (TMS): 8.39 (s, 1H), 8.28 (d, J=5 Hz, 1H), 7.88 (m, 1H), 7.77 (s, 2H), 7.58 (s, 1H), 7.30 (m, 1H), 6.96 (m, 1H), 4.65 (m, 1H), 4.52 (m, 1H), 4.16 (m, 1H), 3.80 (m, 1H), 3.75 (s, 3H), 3.73 (m, 2H), 3.57 (m, 1H), 3.16 (m, 1H), 2.97 (m, 1H), 2.48 (m, 3H), 2.26 (m, 1H), 2.13 (m, 3H), 1.92 (m, 2H), 1.74 (m, 3H), 1.30 (m, 2H).

### EXAMPLE 42

### Methyl (2S)-2-[1-(3,5-dichlorophenylsulfonyl)-L-prolylamino]-5-[4-(2-isopropylaminoimidazo[4,5-c]pyridin-1-ylmethyl)piperidin-1-yl]-5-oxopentanoate

Starting compounds: reference examples 48 and 31; 43% yield.

Obtained as a crude product.

### EXAMPLE 43

### Methyl (2S)-2-[1-(3,5-dichlorophenylsulfonyl)-L-prolylamino]-5-[4-(diethylaminomethyl)piperidin-1-yl]-5-oxopentanoate

Starting compounds: reference examples 48 and 32; 38% yield.

¹H NMR (300 MHz, CDCl₃) δ (TMS): 7.88 (m, 1H); 7.67 (s, 2H), 7.48 (s, 1H), 4.40 (m, 2H), 4.13 (m, 1H), 3.68 (m, 1H), 3.61 (s, 3H), 3.44 (m, 1H), 3.13 (m, 1H), 2.74 (m, 5H), 2.42 (m, 5H), 2.10 (m, 1H), 2.03 (m, 2H), 1.68 (m, 6H), 1.00 (m, 8H).

### EXAMPLE 44

### Methyl (2S)-2-[1-(3,5-dichlorophenylsulfonyl)-L-prolyl]amino-3-[[4-(2-methylimidazo[4,5-c]pyridin-1-ylmethyl)piperidin-1-ylcarbonyl]amino]propionate

To a solution of 2-methyl-1-(4-piperidylmethyl)imidazo[4,5-c]pyridine (obtained in reference example 8) (0.4 g, 1.74 mmol) and DIEA (0.37 mL, 2.12 mmol) in CHCl₃ (13 mL), triphosgene (0.182 g, 0.613 mmol) was slowly added under argon atmosphere, and the resulting mixture was stirred at room temperature for 30 min. Next, methyl (2S)-3-amino-2-[1-(3,5-dichlorophenylsulfonyl)-L-prolyl]aminopropionate hydrochloride (obtained in reference example 52) (0.8 g, 1.74 mmol) and DIEA (1.12 mL, 6.43 mmol) were added and the reaction mixture was stirred overnight at room temperature. It was cooled to 0° C. and treated with 0.2 M NaHCO₃ solution. The phases were separated, the organic phase was dried over sodium sulfate and concentrated to dryness. The crude product obtained was purified by chromatography on silica gel using CHCl₃/MeOH mixtures of increasing polarity as eluent. 340 mg of the title compound of the example was obtained (29% yield).

¹H NMR (300 MHz, CDCl₃) δ (TMS): 9.00 (s, 1H), 8.39 (d, J=5.6 Hz, 1H), 7.80 (s, 2H), 7.74 (m, 1H), 7.65 (m, 1H), 7.25 (d, J=5.6 Hz, 1H), 5.20 (m, 1H), 4.56 (m, 1H), 4.12 (m, 1H), 4.06 (m, 2H), 3.80 (m, 2H), 3.71 (s, 3H), 3.66 (m, 2H), 3.26 (s, 1H), 2.75 (m, 2H), 2.65 (s, 3H), 2.07 (m, 1H), 2.00 (m, 2H), 1.81 (m, 3H), 1.59 (m, 2H), 1.28 (m, 2H).

Following a similar procedure to that described in example 44, but using in each case suitable amines, the following compounds were obtained:

### EXAMPLE 45

### Methyl (2S)-2-[1-(3,5-dichlorophenylsulfonyl)-L-prolyl]amino-3-[[4-(dimethylaminomethyl)piperidin-1-ylcarbonyl]amino]propionate

Compounds used: reference examples 52 and 21; 15% yield.

¹H NMR (300 MHz, CDCl₃) δ (TMS): 7.92 (d, J=6.9 Hz, 1H), 7.75 (s, 2H), 7.57 (s, 1H), 5.18 (m, 1H), 4.49 (m, 1H), 4.13 (m, 1H), 3.90 (m, 2H), 3.74 (s, 3H), 3.67 (m, 1H). 3.60 (m, 2H), 3.20 (m, 1H), 2.97 (m, 2H), 2.18 (s, 3H), 2.12 (s, 3 H), 2.08 (m, 2H), 1.93 (m, 4H), 1.72 (broad d, J=11.4 Hz, 2H), 1.65 (m, 1H), 1.07 (m, 2H).

### EXAMPLE 46

### Methyl (2S)-3-[[(4-(1-piperidylmethyl)piperidin-1-ylcarbonyl]amino]-2-[N-tosyl-L-prolyl]aminopropionate

Compounds used: reference examples 39 and 54; 48% yield.

¹H NMR (300 MHz, CDCl₃) δ (TMS): 7.73 (d, J=8.3 Hz, 2H), 7.67 (m, 1H), 7.33 (d, J=8.3 Hz, 2H), 5.21 (m, 1H), 4.54 (m, 1H), 4.05 (m, 1H), 3.91 (m, 2H), 3.77 (s, 3H), 3.75 (m, 1H), 3.65 (m, 2H), 3.15 (m, 1H), 2.72 (m, 2H), 2.43 (s, 3H), 2.28 (m, 4H), 2.09 (m, 3H), 1.95 (m, 1H), 1.73 (m, 2H), 1.62 (m, 2H), 1.58 (m, 4H), 1.50 (m, 2H), 1.38 (m, 3H).

### EXAMPLE 47

### Methyl (2S)-2-[1-(3,5-dichlorophenylsulfonyl)-L-prolyl]amino-3-[4. (dimethylaminomethyl)piperidin-1-ylcarbonyloxy]propionate

To a solution of N-(3,5-dichlorophenylsulfonyl)-L-proline (obtained in reference example 47) (67 mg, 0.2 mmol) in CHCl₃ (5 mL), EDC-HCl (40 mg, 0.2 mmol), NHS (23 mg, 0.2 mmol) and NMM (0.1 mL, 0.9 mmol) were added under argon atmosphere, and the resulting mixture was stirred at room temperature for 4 h. Next, methyl (2S)-2-amino-3-[[4-(dimethylaminomethyl)piperidin-1-yl]carbonyloxy]propionate dihydrochloride (obtained in reference example 53) (72 mg, 0.2 mmol) dissolved in CHCl₃ (3 mL) and NMM (0.25 mL, 2.3 mmol) were added, and the mixture was further stirred overnight. It was treated with saturated NaHCO₃ solution and extracted 3 times with CHCl₃. The combined organic phases were dried over sodium sulfate and concentrated to dryness, to afford a crude product which was purified by chromatography on silica gel using a CHCl₃/MeOH/NH₃ 60:2:0.1 mixture as eluent. 56 mg of the title compound of the example was obtained (47% yield).

¹H NMR (300 MHz, CDCl₃) δ (TMS): 7.75 (s, 2H), 7.61 (m, 2H), 4.73 (m, 1H), 4.46 (m, 2H), 4.12 (m, 2H), 4.00 (m, 1H), 3.79 (s, 3H), 3.57 (m, 1H). 3.21 (m, 1H), 2.76 (m, 2H), 2.27 (m, 2H), 2.16 (s, 6H), 2.08 (m, 2H), 1.85 (m, 2H), 1.75 (m, 2H), 1.65 (m, 1H), 1.04 (m, 2H).

Following a similar procedure to that described in example 47, but starting in each case from the suitable acid and amine, the following compounds were obtained:

### EXAMPLE 48

### Methyl (2S)-5-[4-[2-[(4-methylpiperazin-1-yl)carbonyloxy]ethyl]piperidin-1-yl]-5-oxo-2-[1-tosyl-L-prolylamino]pentanoate

Starting compounds: reference examples 49 and 35; 38% yield.

¹H NMR (300 MHz, CDCl₃) δ (TMS): 7.75 (d, J=8.1 Hz, 2H), 7.59 (d, J=7.7 Hz, 1H), 7.34 (d, J=8.1 Hz, 2H), 4.55 (m, 2H), 4.11 (m, 3H), 3.80 (m, 1H), 3.75 (s, 3H), 3.55 (m, 1H), 3.47 (m, 4H), 3.25 (m, 1H), 2.90 (m, 1H), 2.49 (m, 1H), 2.46 (s, 3H), 2.40 (m, 4H), 2.34 (m, 4H), 2.10 (m, 2H), 1.60 (m, 10H), 1.10 (m, 2H).

### EXAMPLE 49

### Methyl (2S)-2-[1-(3,5-dichlorophenylsulfonyl)-L-prolyl]amino-5-[4-[(diethylaminocarbonyloxy)methyl]piperidin-1-yl]-5-oxopentanoate

Starting compounds: reference examples 48 and 44; 50% yield.

¹H NMR (300 MHz, CDCl₃) δ (TMS): 7.78 (s, 2H), 7.73 (m, 1H), 7.61 (s, 1H), 4.66 (m, 1H), 4.50 (m, 1H), 4.14 (m, 1H), 3.94 (m, 2H), 3.90 (m, 1H), 3.77 (s, 3H), 3.60 (m, 1H), 3.24 (m, 5H), 3.02 (m, 1H), 2.51 (m, 1H), 2.47 (m, 2H), 2.22 (m, 1H), 2.17 (m, 2H), 1.90 (m, 6H), 1.20 (m, 2H), 1.12 (m, 6H).

### EXAMPLE 50

### Methyl (2S)-2-[1-(3,5-dichlorophenylsulfonyl)-L-prolylamino]-5-[4-[(4-methylpiperazin-1-yl)carbonyloxymethyl]piperidin-1-yl]-5-oxopentanoate

Starting compounds: reference examples 48 and 45; 75% yield.

¹H NMR (300 MHz, CDCl₃) δ (TMS): 7.77 (s, 2H), 7.67 (m, 1H), 7.60 (s, 1H), 4.63 (m, 1H), 4.51 (m, 1H), 4.13 (m, 1H), 3.96 (m, 2H), 3.81 (m, 1H), 3.78 (s, 3H), 3.60 (m, 1H), 3.48 (m, 4H), 3.24 (m, 1H), 2.98 (m, 1H), 2.48 (m, 1H), 2.46 (m, 1H), 2.42 (m, 2H), 2.35 (m, 4H), 2.30 (s, 3H), 2.16 (m, 2H), 1.92 (m, 2H), 1.81 (m, 4H), 1.21 (m, 2H).

### EXAMPLE 51

### Methyl (2S)-2-[1-(3,5-dichlorophenylsulfonyl)-L-prolylamino]-5-oxo-5-[444-pyridyloxymethyl)piperidin-1-yl]pentanoate

Starting compounds: reference examples 48 and 46; 85% yield.

¹H NMR (300 MHz, CDCl₃) δ (TMS): 8.41 (d, J=5.1 Hz, 2H), 7.76 (s, 2H), 7.59 (s, 1H), 7.46 (m, 1H), 6.77 (d, J=5.1 Hz, 2H), 4.69 (m, 1H), 4.51 (m, 1H), 4.13 (m, 1H), 3.86 (m, 3H), 3.76 (s, 3H), 3.58 (m, 1H), 3.15 (m, 1H), 3.05 (m, 1H), 2.51 (m, 1H), 2.49 (m, 2H), 2.30 (m, 1H), 2.18 (m, 2H), 2.00 (m, 1H), 1.90 (m, 5H), 1.30 (m, 2H).

### EXAMPLE 52

### Methyl (2S)-5-[4-(4-methylpiperazin-1-ylmethyl)piperidin-1-yl]-5-oxo-2-[1-tosyl-L-prolylamino]pentanoate

Starting compounds: reference examples 49 and 27; 33% yield.

¹H NMR (300 MHz, CDCl₃) δ (TMS): 7.75 (d, J=8.3 Hz, 2H), 7.60 (m, 1H), 7.34 (d, J=8.3 Hz, 2H), 4.54 (m, 2H), 4.09 (m, 1H), 3.82 (m, 1H), 3.79 (s, 3H), 3.55 (m, 1H), 3.19 (m, 1H), 3.00 (m, 1H), 2.48 (m, 12H), 2.44 (s, 3H), 2.27 (s, 3H), 2.11 (m, 4H), 1.82 (m, 2H), 1.60 (m, 4H), 1.25 (m, 2H).

### EXAMPLE 53

### Methyl (2S)-5-[4-(dimethylaminomethyl)piperidin-1-yl]-5-oxo-2-[1-tosyl-L-prolylamino]pentanoate

Starting compounds: reference examples 49 and 21; 40% yield.

¹H NMR (300 MHz, CDCl₃) δ (TMS): 7.78 (d, J=8.3 Hz, 2H), 7.65 (m, 1H), 7.38 (d, J=8.3 Hz, 2H), 4.62 (m, 2H), 4.13 (m, 1H), 3.87 (m, 1H), 3.80 (s, 3H), 3.58 (m, 1H), 3.22 (m, 1H), 3.01 (m, 1H), 2.48 (m, 2H), 2.44 (s, 3H), 2.30 (m, 1H), 2.22 (s, 6H), 2.15 (m, 3H), 2.11 (m, 2H), 1.85 (m, 2H), 1.65 (m, 4H), 1.15 (m, 2H).

### EXAMPLE 54

### (2S)-5-[4-[(1-Oxoisoindolin-2-yl)methyl]piperidin-1-yl]-2-[1-(3,5-dichlorophenylsulfonyl)-L-prolylamino]-5-oxopentanoic Acid

To a solution of methyl (2S)-2-[1-(3,5-dichlorophenylsulfonyl)-L-prolylamino]-5-oxo-5-[4-(1-oxoisoindolin-2-ylmethyl)piperidin-1-yl]pentanoate (obtained in example 3) (300 mg, 0.47 mmol) in THF (8 mL), LiOH.H₂O (45 mg, 1.07 mmol) dissolved in H₂O (8 mL) was added. The reaction mixture was stirred overnight at room temperature and THF was evaporated. The resulting residue was cooled to 0° C. and was acidified by adding 1 N HCl until reaching the maximal turbidity the solution. The solid obtained was collected by filtration and dried. The title compound of the example was obtained as a white solid (763 mg, 79% yield).

¹H NMR (300 MHz, CD₃OD) δ (TMS): 7.86 (s, 2H), 7.76 (m, 2H), 7.54 (m, 3H), 4.84 (s, 2H), 4.51 (s, 2H), 4.47 (m, 2H), 4.22 (m, 1H), 3.98 (m, 1H), 3.53 (m, 3H), 3.29 (m, 1H), 3.04 (m, 1H), 2.61 (m, 3H), 2.30 (m, 1H), 1.90-2.10 (complex signal, 5H), 1.69 (m, 3H), 1.20 (m, 2H).

MS: m/z 665 (MH⁺).

Following a similar procedure to that described in example 54, but starting in each case from a suitable ester, the following compounds were obtained:

### EXAMPLE 55

### (2S)-2-[1-(3,5-Dichlorophenylsulfonyl)-L-prolylamino]-5-[4-[(3-methylbutanoylamino)methyl]piperidin-1-yl]-5-oxopentanoic Acid

Starting compound: example 13; 61% yield.

¹H NMR (300 MHz, CD₃OD) δ (TMS): 7.88 (s, 2H), 7.81 (s, 1H), 4.84 (s, 3H), 4.47 (m, 2H), 4.23 (m, 1H), 3.98 (m, 1H), 3.55 (m, 1H), 3.03 (m, 3H), 2.57 (m, 3H), 2.25 (m, 1H), 1.90-2.10 (complex signal, 9H), 1.72 (m, 3H), 1.20 (m, 2H), 0.91 (m, 6H).

MS: m/z 633 (MH⁺).

### EXAMPLE 56

### (2S)-5-[4-[(N′-tert-Butylureido)methyl]piperidin-1-yl]-2-[1-(3,5-dichlorophenylsulfonyl)-L-prolylamino]-5-oxopentanoic Acid

Starting compound: example 14; 69% yield.

¹H NMR (300 MHz, CD₃OD) δ (TMS): 7.88 (s, 2H), 7.80 (s, 1H), 4.85 (s, 4H), 4.48 (m, 2H), 4.23 (m, 1H), 3.96 (m, 1H), 3.54 (m, 1H), 3.03 (m, 3H), 2.60 (m, 3H), 1.98 (m, 5H), 1.59 (m, 5H), 1.32 (s, 9H), 1.21 (m, 2H).

MS: m/z 648 (MH⁺).

### EXAMPLE 57

### (2S)-2-[1-(3,5-Dichlorophenylsulfonyl)-L-prolylamino]-5-[4-[(isobutoxycarbonylamino)methyl]piperidin-1-yl]-5-oxopentanoic Acid

Starting compound: example 15; 65% yield.

¹H NMR (300 MHz, CD₃OD) δ (TMS): 7.88 (s, 2H), 7.80 (s, 1H), 4.84 (s, 3H), 4.48 (m, 2H), 4.22 (m, 1H), 3.96 (m, 1H), 3.78 (m, 2H), 3.54 (m, 1H), 2.99 (m, 3H), 2.57 (m, 3H), 2.25 (m, 1H), 1.0-1.98 (complex signal, 12H), 0.92 (m, 6H).

MS: m/z 646 (MH⁻).

### EXAMPLE 58

### (2S)-2-[1-(3,5-Dichlorophenylsulfonyl)-L-prolylamino]-5-[4-[(N′-isopropylthioureido)methyl]piperidin-1-yl]-5-oxopentanoic Acid

Starting compound: example 18; 63% yield.

¹H NMR (300 MHz, CD₃OD) δ (TMS): 7.88 (s, 2H), 7.80 (s, 1H), 4.85 (s, 4H), 4.49 (m, 2H), 4.23 (m, 2H), 4.05 (m, 1H), 3.53 (m, 1H), 3.30 (m, 2H), 3.01 (m, 1H), 2.58 (m, 3H), 2.26 (m, 1H), 2.01 (m, 5H), 1.71 (m, 4H), 1.15 (m, 8H).

MS: m/z 650 (MH⁺).

### EXAMPLE 59

### (2S)-2-[1-(3,5-Dichlorophenylsulfonyl)-L-prolylamino]-5-[4-[2-(2-ethyl-5,7-dimethylimidazo[4,5-b]pyridin-3-yl)ethyl]piperidin-1-yl]-5-oxopentanoic Acid

Starting compound: example 19; 59% yield.

MS: m/z 721 (MH⁺).

### EXAMPLE 60

### (2S)-2-[1-(3,5-Dichlorophenylsulfonyl)-L-prolylamino]-5-[4-[(isopropylsulfonylamino)methyl]piperidin-1-yl]-5-oxopentanoic Acid

Starting compound: example 17; 59% yield.

¹H NMR (300 MHz, CD₃OD) δ (TMS): 7.88 (s, 2H), 7.81 (s, 1H), 4.83 (s, 3H), 4.49 (m, 2H), 4.22 (m, 1H), 3.99 (m, 1H), 3.55 (m, 1H), 3.18 (m, 1H), 3.01 (m, 1H), 2.93 (m, 2H), 2.62 (m, 3H), 2.32 (m, 1H), 1.99 (m, 4H), 1.77 (m, 5H), 1.29 (m, 6H), 1.05 (m, 2H).

MS: m/z 655 (MH⁺).

### EXAMPLE 61

### (2S)-2-[1-(3,5-Dichlorophenylsulfonyl)-L-prolylamino]-5-oxo-5-[4-[2-(pyrrolidin-1-ylcarbonyloxy)ethyl]piperidin-1-yl]pentanoic Acid

Starting compound: example 20; 60% yield.

¹H NMR (300 MHz, CD₃OD) δ (TMS): 7.89 (s, 2H), 7.81 (s, 1H), 4.85 (s, 2H), 4.50 (m, 2H), 4.25 (m, 1H), 4.11 (m, 2H), 3.96 (m, 1H), 3.54 (m, 1H), 3.30 (m, 5H), 3.01 (m, 1H), 2.58 (m, 3H), 2.27 (m, 1H), 2.01 (m, 4H), 1.87 (m, 4H), 1.73 (m, 4H), 1.55 (m, 2H), 1.21 (m, 2H).

MS: m/z 661 (MH⁺).

### EXAMPLE 62

### (2S)-5-Oxo-5-[4-[(2-oxopyrrolidin-1-yl)methyl]piperidin-1-yl]-2-[1-tosyl-L-prolylamino]pentanoic Acid

Starting compound: example 6; 85% yield.

¹H NMR (300 MHz, CD₃OD) δ (TMS): 7.78 (d, J=8.1 Hz, 2H), 7.43 (d, J=8.1 Hz, 2H), 4.82 (s, 2H), 4.44 (m, 2H), 4.13 (m, 1H), 3.98 (m, 1H), 3.05-3.55 (complex signal, 7H), 2.54 (m, 3H), 2.44 (s, 3H), 2.34 (m, 3H), 1.75-2.1 (complex signal, 7H), 1.65 (m, 3H), 1.15 (m, 2H).

MS: m/z 563 (MH⁺).

### EXAMPLE 63

### (2S)-5-Oxo-5-[4-(2-phenylimidazol-1-ylmethyl)piperidin-1-yl]-2-[1-tosyl-L-prolylamino]pentanoic Acid

Starting compound: example 7; 89% yield.

¹H NMR (300 MHz, CD₃OD) δ (TMS): 7.78 (d, J=8.2 Hz, 2H), 7.40-7.60 (complex signal, 6H), 7.43 (d, J=8.2 Hz, 2H), 7.38 (m, 1H), 4.82 (s, 2H), 4.42 (m, 2H), 3.9-4.1 (complex signal, 4H), 3.46 (m, 1H), 3.15 (m, 1H), 2.93 (m, 1H), 2.51 (m, 5H), 2.26 (m, 1H), 1.60-1.90 (complex signal, 9H), 1.21 (m, 2H).

MS: m/z 622 (MH⁺).

### EXAMPLE 64

### (2S)-5-Oxo-2-[1-tosyl-L-prolyl]amino-5-[4-[[4-(trifluoromethyl)pyrimidin-2-yl]aminomethyl]piperidin-1-yl]pentanoic Acid

Starting compound: example 16; 45% yield.

¹H NMR (300 MHz, CD₃OD) δ (TMS): 8.86 (m, 1H), 7.78 (d, J=8.2 Hz, 2H), 7.42 (d, J=8.2 Hz, 2H), 6.85 (m, 1H), 4.83 (s, 3H), 4.46 (m, 2H), 4.13 (m, 1H), 3.98 (m, 1H), 3.66 (m, 1H), 3.55 (m, 2H), 3.23 (m, 1H), 3.05 (m, 1H), 2.61 (m, 1H), 2.56 (m, 2H), 2.44 (s, 3H), 2.30 (m, 1H), 1.7-2.05 (complex signal, 7H), 1.58 (m, 1H), 1.22 (m, 2H).

MS: m/z 641 (MH⁺).

### EXAMPLE 65

### (2S)-2-[1-(3,5-Dichlorophenylsulfonyl)-L-prolylamino]-5-[4-(2-ethyl-5,7-dimethylimidazo[4,5-b]pyridin-3-ylmethyl)piperidin-1-yl-5-oxopentanoic Acid

Starting compound: example 4; 79% yield.

¹H NMR (300 MHz, CD₃OD) δ (TMS): 7.85 (s, 2H), 7.76 (s, 1H), 6.96 (s, 1H), 4.78 (s, 2H), 4.47 (m, 2H), 4.19 (m, 3H), 3.95 (m, 1H), 3.66 (m, 1H), 3.55 (m, 2H), 2.96 (m, 2H), 2.56 (s, 3H), 2.55 (s, 3H), 2.53 (m, 3H), 2.28 (m, 2H), 2.00 (m, 4H), 1.69 (m, 2H), 1.57 (m, 1H), 1.21-1.42 (complex signal, 5H).

MS: m/z 707 (MH⁺).

### EXAMPLE 66

### (2S)-2-[143,5-Dichlorophenylsulfonyl)-L-prolylamino]-5-[4-[2-[(2-methoxyethyl)aminocarbonyloxy]ethyl]piperidin-1-yl]-5-oxopentanoic Acid

Starting compound: example 27; 62% yield.

MS: m/z 665 (MH⁺).

### EXAMPLE 67

### (2S)-2-[1-(3,5-Dichlorophenylsulfonyl)-L-prolylamino]-5-[4-(4-morpholinylcarbonylaminomethyl)piperidin-1-yl]-5-oxopentanoic Acid

Starting compound: example 28; 64% yield.

¹H NMR (300 MHz, CD₃OD) δ (TMS): 7.88 (s, 2H), 7.81 (s, 1H), 4.83 (s, 3H), 4.49 (m, 2H), 4.25 (m, 1H), 4.00 (m, 1H), 3.63 (m, 4H), 3.51 (m, 1H), 3.30 (m, 4H), 3.04 (m, 4H), 2.60 (m, 3H), 2.25 (m, 1H), 1.99 (m, 4H), 1.70 (m, 4H), 1.10 (m, 2H).

MS: m/z 662 (MH⁺).

### EXAMPLE 68

### (2S)-2-[1-(3,5-Dichlorophenylsulfonyl)-L-prolylamino]-5-oxo-5-[4-(1-pyrrolidinylcarbonylaminomethyl)piperidin-1-yl]pentanoic Acid

Starting compound: example 29; 30% yield.

¹H NMR (300 MHz, CD₃OD) δ (TMS): 7.88 (s, 2H), 7.81 (s, 1H), 4.83 (s, 3H), 4.55 (m, 2H), 4.27 (m, 1H), 4.00 (m, 1H), 3.53 (m, 1H), 3.30 (m, 4H), 3.01 (m, 4H), 2.60 (m, 2H), 2.25 (m, 1H), 2.00 (m, 10H), 1.88 (m, 3H), 1.12 (m, 2H).

MS: m/z 646 (MH⁺).

### EXAMPLE 69

### (2S)-2-[1-(3,5-Dichlorophenylsulfonyl)-L-prolyl]amino-5-[4-[(diethylaminocarbonyloxy)methyl]piperidin-1-yl]-5-oxopentanoic Acid

Starting compound: example 49; 72% yield.

¹H NMR (300 MHz, CD₃OD) δ (TMS): 7.89 (s, 2H), 7.80 (s, 1H), 4.83 (s, 2H), 4.54 (m, 1H), 4.48 (m, 1H), 4.22 (m, 1H), 4.01 (m, 1H), 3.92 (m, 2H), 3.54 (m, 1H), 3.30 (m, 5H), 3.00 (m, 1H), 2.59 (m, 3H), 2.22 (m, 1H), 2.00 (m, 6H), 1.75 (m, 2H), 1.25 (m, 2H), 1.09 (m, 6H).

MS: m/z 646 (MH⁺).

### EXAMPLE 70

### (2S)-2-[1-(3,5-Dichlorophenylsulfonyl)-L-prolylamino]-5-oxo-5-[4-(2-oxooxazolidin-3-ylmethyl)piperidin-1-yl]pentanoic Acid

To a solution of methyl (2S)-2-[1-(3,5-dichlorophenylsulfonyl)-L-prolylamino]-5-oxo-5-[4-(2-oxooxazolidin-3-ylmethyl)piperidin-1-yl]pentanoate (obtained in example 1) (300 mg, 0.47 mmol) in THF (8 mL), LiOH.H₂O (45 mg, 1.07 mmol) dissolved in H₂O (8 mL) was added. The reaction mixture was stirred overnight at room temperature and THF was evaporated. The resulting residue was cooled to 0° C. and brought to pH=2 by the addition of 1 N HCl. It was concentrated to dryness and the crude product obtained was purified by chromatography on silica gel using CHCl₃/MeOH 5-30% mixtures as eluent. The title compound of the example was obtained as a white solid (155 mg, 53% yield).

¹H NMR (300 MHz, CD₃OD) 5 (TMS): 7.78 (s, 2H), 7.79 (s, 1H), 4.78 (s, 2H), 4.55 (m, 1H), 4.45 (m, 1H), 4.33 (m, 2H), 4.24 (m, 1H), 4.01 (m, 1H), 3.45-3.70 (m, 5H), 3.11 (m, 2H), 2.60 (m, 2H), 2.28 (m, 1H), 1.1-2.2 (complex signal, 11H).

MS: m/z 619 (MH⁺).

Following a similar procedure to that described in example 70, but starting in each case from a suitable ester, the following compounds were obtained:

### EXAMPLE 71

### (2S)-2-[1-(3,5-Dichlorophenylsulfonyl)-L-prolylamino]-5-[4-(2-oxopyrrolidin-1-ylmethyl)piperidin-1-yl]-5-oxopentanoic Acid

Starting compound: example 2; 19% yield.

¹H NMR (300 MHz, CD₃OD) δ (TMS): 7.87 (s, 2H), 7.79 (s, 1H), 4.78 (s, 2H), 4.46 (m, 2H), 4.25 (m, 1H), 3.94 (m, 1H), 3.40-3.60 (complex signal, 3H), 3.30 (m, 2H), 3.14 (m, 1H), 3.04 (m, 1H), 2.59 (m, 3H), 2.36 (m, 3H), 2.00-2.10 (complex signal, 7H), 1.66 (m, 3H), 1.21 (m, 2H).

MS: m/z 617 (MH⁺).

### EXAMPLE 72

### (2S)-2-[1-(3,5-Dichlorophenylsulfonyl)-L-prolylamino]-5-[4-(2-methylimidazo[4,5-c]pyridin-1-ylmethyl)piperidin-1-yl]-5-oxopentanoic Acid

Starting compound: example 35; 17% yield.

¹H NMR (300 MHz, CD₃OD) δ (TMS): 8.81 (s, 1H), 8.28 (d, J=5.7 Hz, 1H), 7.85 (s, 2H), 7.78 (s, 1H), 7.66 (m, 1H), 4.57 (m, 1H), 4.87 (s, 2H), 4.45 (m, 1H), 4.18 (m, 2H), 3.98 (m, 1H), 3.52 (m, 1H), 3.27 (m, 1H), 2.97 (m, 1H), 2.66 (s, 3H), 2.51 (m, 2H), 2.35 (m, 1H), 2.25 (m, 1H), 1.98 (m, 4H), 1.2-1.85 (complex signal, 7H).

MS: m/z 665 (MH⁺).

### EXAMPLE 73

### (2S)-5-[4-(2-Methylimidazo[4,5-c]pyridin-1-ylmethyl)piperidin-1-yl]-5-oxo-2-[1-tosyl-L-prolylamino]pentanoic Acid

Starting compound: example 5; 23% yield.

¹H NMR (300 MHz, CD₃OD+CDCl₃) δ (TMS): 9.04 (s, 1H), 8.45 (m, 1H), 8.09 (m, 1H), 7.73 (d, J=7.9 Hz, 2H), 7.37 (d, J=7.9 Hz, 2H), 4.63 (m, 1H), 4.52 (m, 1H), 4.44 (s, 2H), 4.28 (m, 1H), 4.07 (m, 1H), 3.95 (m, 1H), 3.57 (m, 1H), 3.15 (m, 1H), 2.95 (m, 1H), 2.77 (m, 3H), 2.40-2.65 (complex signal, 3H), 2.44 (s, 3H), 1.9-2.30 (complex signal, 5H), 1.20-1.80 (complex signal, 7H).

MS: m/z 611 (MH⁺).

### EXAMPLE 74

### (2S)-5-[4-[[1-(2-Ethoxyethyl)benzimidazol-2-yl]methyl]piperazin-1-yl]-5-oxo-2-[[1-tosyl-L-prolyl]amino]pentanoic Acid

Starting compound: example 8; 10% yield.

¹H NMR (300 MHz, CD₃OD) δ (TMS): 7.76 (d, J=8.3 Hz, 2H), 7.57 (m, 2H), 7.39 (d, J=8.3 Hz, 2H), 7.30 (m, 2H), 4.83 (s, 2H), 4.59 (m, 2H), 4.45 (m, 1H), 4.12 (m, 1H), 3.91 (s, 2H), 3.79 (m, 2H), 3.2-3.6 (complex signal, 8H), 2.53 (m, 4H), 2.41 (s, 3H), 2.25 (m, 2H), 1.93 (m, 2H), 1.80 (m, 2H), 1.56 (m, 2H), 1.04 (t, J=7 Hz, 3H).

MS: m/z 669 (MH⁺).

### EXAMPLE 75

### (2S)-5-Oxo-5-[4-(2-pyridylmethyl)piperazin-1-yl]-2-[[1-tosyl-L-prolyl]amino]pentanoic Acid

Starting compound: example 9; 47% yield.

¹H NMR (300 MHz, CD₃OD) δ (TMS): 8.50 (m, 1H), 7.82 (m, 1H), 7.78 (d, J=8.3 Hz, 2H), 7.52 (m, 1H), 7.42 (d, J=8.3 Hz, 2H), 7.33 (m, 1H), 4.83 (s, 2H), 4.41 (m, 1H), 4.11 (m, 1H), 3.81 (s, 2H), 3.70 (m, 4H), 3.55 (m, 1H), 3.20 (m, 1H), 2.65 (m, 4H), 2.55 (m, 2H), 2.44 (s, 3H), 2.30 (m, 1H), 1.92 (m, 4H), 1.56 (m, 1H).

MS: m/z 558 (MH⁺).

### EXAMPLE 76

### (2S)-5-Oxo-5-[4-(1-oxoisoindolin-2-ylmethyl)piperidin-1-yl]-2-[[1-tosyl-L-prolyl]amino]pentanoic Acid

Starting compound: example 10; 48% yield.

¹H NMR (300 MHz, CD₃OD) δ (TMS): 7.77 (d, J=8.2 Hz, 2H), 7.73 (m, 1H), 7.55 (m, 3H), 7.41 (d, J=8.2 Hz, 2H), 4.84 (s, 2H), 4.51 (m, 3H), 4.45 (m, 1H), 4.35 (m, 1H), 3.99 (m, 1H), 3.66 (m, 1H), 3.53 (m, 2H), 3.23 (m, 1H), 3.02 (m, 1H), 2.52-2.64 (complex signal, 3H), 2.43 (s, 3H), 2.25 (m, 1H) 1.15-2.00 (complex signal, 10H).

MS: m/z 611 (MH⁺).

### EXAMPLE 77

### (2S)-5-Oxo-5-[4-(2-thienylmethyl)piperazin-1-yl]-2-[[1-tosyl-L-prolyl]amino]pentanoic Acid

Starting compound: example 11; 63% yield.

¹H NMR (300 MHz, CD₃OD) δ (TMS): 7.79 (d, J=8.2 Hz, 2H), 7.43 (d, J=8.2 Hz, 2H), 7.29 (m, 1H), 6.93 (m, 2H), 4.84 (s, 2H), 4.56 (m, 1H), 4.29 (m, 1H), 4.18 (m, 1H), 4.11 (m, 1H), 3.74 (s, 2H), 3.23-3.65 (complex signal, 3H), 3.07 (m, 1H), 2.44 (s, 3H), 2.41 (m, 6H), 2.21 (m, 1H), 1.90 (m, 3H), 1.60 (m, 2H).

MS: m/z 563 (MH⁺).

### EXAMPLE 78

### (2S)-5-[4-[(3-Carboxypropionylamino)methyl]piperidin-1-yl]-5-oxo-2-[[1-tosyl-L-prolyl]amino]pentanoic Acid

Starting compound: example 12; 39% yield.

¹H NMR (300 MHz, CD₃OD) δ (TMS): 7.79 (d, J=8.2 Hz, 2H), 7.44 (d, J=8.2 Hz, 2H), 4.89 (s, 4H), 4.52 (m, 2H), 4.12 (m, 1H), 4.00 (m, 1H), 3.67 (m, 1H), 3.54 (m, 2H), 3.25 (m, 2H), 3.05 (m, 2H), 2.60 (m, 4H), 2.45 (s, 3H), 2.25 (m, 2H), 2.17 (m, 1H), 1.2-2.00 (complex signal, 6H), 1.55 (m, 1H), 1.20 (m, 2H).

MS: m/z 595 (MH⁺).

### EXAMPLE 79

### (2S)-2-[1-(3,5-Dichlorophenylsulfonyl)-L-prolylamino]-5-[4-(4-methylpiperazin-1-ylmethyl)piperidin-1-yl]-5-oxopentanoic Acid

Starting compound: example 21; 56% yield.

¹H NMR (300 MHz, CD₃OD) δ (TMS): 7.88 (s, 2H), 7.80 (s, 1H), 4.86 (s, 2H), 4.47 (m, 1H), 4.24 (m, 2H), 3.97 (m, 1H), 3.66 (m, 1H), 3.01 (m, 1H), 2.66 (m, 11H), 2.56 (m, 2H), 2.27 (s, 3H), 1.98 (m, 5H), 1.68 (m, 5H), 1.09 (m, 2H).

MS: m/z 632 (MH⁺).

### EXAMPLE 80

### (2S)-2-[1-(3,5-Dichlorophenylsulfonyl)-L-prolylamino]-5-[4-[2-(4-morpholinyl)ethyl]piperidin-1-yl]-5-oxopentanoic Acid

Starting compound: example 22; 76% yield.

¹H NMR (300 MHz, CD₃OD) δ (TMS): 7.89 (s, 2H), 7.80 (s, 1H), 4.83 (s, 2H), 4.50 (m, 1H), 4.32 (m, 1H), 4.26 (m, 1H), 3.99 (m, 1H), 3.73 (m, 4H), 3.45 (m, 1H), 3.31 (m, 1H), 3.01 (m, 1H), 2.70 (m, 4H), 2.61 (m, 2H), 2.48 (m, 2H), 2.25 (m, 1H), 2.01 (m, 4H), 1.70 (m, 3H), 1.55 (m, 3H), 1.27 (m, 3H).

MS: m/z 633 (MH⁺).

### EXAMPLE 81

### (2S)-2-[1-(3,5-Dichlorophenylsulfonyl)-L-prolylamino]-5-oxo-5-[4-(1-pyrrolylmethyl)piperidin-1-yl]pentanoic Acid

Starting compound: example 36; 57% yield.

¹H NMR (300 MHz, CDCl₃) δ (TMS): 7.98 (m, 1H), 7.76 (s, 2H), 7.59 (s, 1H), 6.62 (m, 2H), 6.14 (m, 2H), 4.70 (m, 1H), 4.30 (m, 1H), 4.12 (m, 1H), 3.80 (m, 1H), 3.75 (m, 2H), 3.62 (m, 1H), 3.30 (m, 1H), 3.05 (m, 1H), 2.63 (m, 3H), 2.25 (m, 1H), 1.92 (m, 4H), 1.70 (m, 4H), 1.25 (m, 2H).

### EXAMPLE 82

### (2S)-2-[1-(3,5-Dichlorophenylsulfonyl)-L-prolylamino]-5-[4-(2,5-dimethylpyrrol-1-ylmethyl)piperidin-1-yl]-5-oxopentanoic Acid

Starting compound: example 37; 6% yield.

¹H NMR (300 MHz, CDCl₃) δ (TMS): 7.83 (m, 1H), 7.76 (s, 2H), 7.59 (s, 1H), 5.76 (d, J=1.2 Hz, 2H), 4.69 (broad d, J=12.8 Hz, 1H), 4.42 (m, 1H), 4.16 (m, 1H), 3.88 (m, 1H), 3.60 (m, 3H), 3.26 (m, 1H), 2.98 (m, 2H), 2.53 (m, 2H), 2.25 (m, 1H), 2.16 (s, 6H), 1.99 (m, 3H), 1.80 (m, 4H), 1.62 (m, 2H), 1.22 (m, 2H).

MS: m/z 627 (MH⁺).

### EXAMPLE 83

### (2S)-2-[1-(3,5-Dichlorophenylsulfonyl)-L-prolylamino]-5-[4-(dimethylaminomethyl)piperidin-1-yl]-5-oxopentanoic Acid

Starting compound: example 38; 81% yield.

¹H NMR (300 MHz, CD₃OD) δ (TMS): 7.89 (s, 2H), 7.81 (s, 1H), 4.85 (s, 2H), 4.54 (m, 1H), 4.30 (m, 1H), 4.24 (m, 1H), 4.02 (m, 1H), 3.55 (m, 1H), 3.35 (m, 1H), 3.10 (m, 1H), 2.93 (m, 3H), 2.83 (s, 6H), 2.50 (m, 3H), 2.22 (m, 1H), 2.05 (m, 4H), 1.79 (m, 3H), 1.10 (m, 2H).

MS: m/z 577 (MH⁺).

### EXAMPLE 84

### (2S)-2-[1-(3,5-Dichlorophenylsulfonyl)-L-prolylamino]-5-[4-[2-(dimethylamino)ethyl]piperazin-1-yl]-5-oxopentanoic Acid

Starting compound: example 39; 25% yield.

¹H NMR (300 MHz, CD₃OD) δ (TMS): 7.90 (s, 2H), 7.81 (s, 1H), 4.85 (s, 2H), 4.28 (m, 2H), 3.58 (m, 5H), 3.43 (m, 4H), 2.5-3 (m, 7H), 2.58 (s, 6H), 2.25 (m, 1H), 2.01 (m, 5H).

MS: m/z 592 (MH⁺).

### EXAMPLE 85

### (2S)-2-[1-(3,5-Dichlorophenylsulfonyl)-L-prolylamino]-5-[4-(2-ethylimidazo[4,5-c]pyridin-1-ylmethyl)piperidin-1-yl]-5-oxopentanoic Acid

Starting compound: example 40; 55% yield.

¹H NMR (300 MHz, CD₃OD) δ (TMS): 8.99 (s, 1H), 8.40 (m, 1H), 7.96 (m, 1H), 7.85 (s, 2H), 7.79 (s, 1H), 4.94 (s, 2H), 4.57 (m, 1H), 4.47 (m, 1H), 4.42 (m, 1H), 4.12 (m, 2H), 3.99 (m, 1H), 3.56 (m, 1H), 3.30 (m, 1H), 3.02 (m, 3H), 2.56 (m, 3H), 2.25 (m, 2H), 1.99 (m, 4H). 1.65-1.15 (complex signal, 8H).

MS: m/z 677 (MH⁺).

### EXAMPLE 86

### (2S)-2-[1-(3,5-Dichlorophenylsulfonyl)-L-prolylamino]-5-oxo-5-[4-(2-oxo-2,3-dihydroimidazo[4,5-c]pyridin-1-ylmethyl)piperidin-1-yl]pentanoic Acid

Starting compound: example 41; 59% yield.

¹H NMR (300 MHz, CD₃OD) δ (TMS): 8.27 (s, 1H), 8.22 (d, J=5.5 Hz, 1H), 7.94 (s, 2H), 7.84 (s, 1H), 7.30 (m, 1H), 4.91 (s, 3H), 4.60 (m, 1H), 4.38 (m, 1H), 4.30 (m, 1H), 4.07 (m, 1H), 3.86 (m, 2H), 3.62 (m, 1H), 3.37 (m, 1H), 3.08 (m, 1H), 2.63 (m, 3H), 2.29 (m, 1H), 2.06 (m, 4H), 1.73 (m, 3H), 1.3-1.5 (m, 3H).

MS: m/z 667 (MH⁺).

### EXAMPLE 87

### (2S)-2-[1-(3,5-Dichlorophenylsulfonyl)-L-prolylamino]-5-[(2-isopropylaminoimidazo[4,5-c]pyridin-1-ylmethyl)piperidin-1-yl]-5-oxopentanoic Acid

Starting compound: example 42; 36% yield.

¹H NMR (300 MHz, CD₃OD) δ (TMS): 8.41 (s, 1H), 8.07 (m, 1H), 7.86 (s, 2H), 7.78 (s, 1H), 7.36 (m, 1H), 4.86 (s, 3H), 4.53 (m, 1H), 4.29 (m, 1H), 4.23 (m, 2H), 4.20 (m, 1H), 3.97 (m, 2H), 3.54 (m, 1H), 3.32 (m, 1H), 2.98 (m, 1H), 2.49 (m, 3H), 2.25 (m, 1H), 1.99 (m, 6H), 1.58 (m, 2H), 1.30 (m, 8H).

MS: m/z 708 (MH⁺).

### EXAMPLE 88

### (2S)-2-[1-(3,5-Dichlorophenylsulfonyl)-L-prolylamino]-5-[4-(diethylaminomethyl)piperidin-1-yl]-5-oxopentanoic Acid

Starting compound: example 43; 66% yield.

¹H NMR (300 MHz, CDCl₃) δ (TMS): 7.80 (m, 1H), 7.76 (s, 2H), 7.62 (s, 1H), 4.58 (m, 1H), 4.27 (m, 1H), 4.12 (m, 1H), 3.90 (m, 1H), 3.62 (m, 1H), 3.37 (m, 1H), 3.18 (m, 5H), 2.79 (m, 2H), 2.65 (m, 1H), 2.45 (m, 2H), 2.16 (m, 7H), 1.80 (m, 2H), 1.18 (m, 8H).

MS: m/z 605 (MH⁺).

### EXAMPLE 89

### (2S)-2-[1-(3,5-Dichlorophenylsulfonyl)-L-prolyl]amino-3-[[4-(2-methylimidazo[4,5-c]pyridin-1-ylmethyl)piperidin-1-ylcarbonyl]amino]propionic Acid

Starting compound: example 44; 42% yield.

¹H NMR (300 MHz, CD₃OD+CDCl₃) δ (TMS): 8.80 (s, 1H), 8.25 (d, J=5.6 Hz, 1H), 7.76 (s, 2H), 7.61 (s, 1H), 7.33 (d, J=5.6 Hz, 1H), 4.16 (s, 3H), 4.01 (m, 1H), 3.73 (m, 5H), 3.65 (m, 2H), 3.12 (m, 1H), 2.60 (m, 2H), 2.59 (s, 3H), 1.84 (m, 4H), 1.58 (m, 1H), 1.44 (m, 1H), 1.15 (m, 2H), 0.95 (m, 2H).

MS: m/z 666 (MH⁺).

### EXAMPLE 90

### (2S)-2-[1-(3,5-Dichlorophenylsulfonyl)-L-prolylamino]-5-[4-[[(dimethylaminoacetyl)amino]methyl]piperidin-1-yl]-5-oxopentanoic Acid

Starting compound: example 23; 51% yield.

¹H NMR (300 MHz, TFA+DMSO-d₆) δ (TMS): 9.61 (broad s, 1H), 8.45 (s, 1H), 8.35 (d, J=7.6 Hz, 1H), 7.96 (s, 1H), 7.85 (s, 2H), 4.35 (broad d, J=13.2 Hz, 2H), 4.25 (m, 2H), 3.86 (m, 2H), 3.77 (m, 2H), 3.35 (m, 1H), 3.22 (m, 1H), 2.98 (m, 3H), 2.76 (s, 6H), 2.35 (m, 2H), 2.02 (m, 1H), 1.82 (m, 4H), 1.62 (m, 3H), 1.12 (m, 2H).

MS: m/z 634 (MH⁺).

### EXAMPLE 91

### (2S)-2-[1-(3,5-Dichlorophenylsulfonyl)-L-prolylamino]-5-[4-[2-(diethylamino)ethyl]piperidin-1-yl]-5-oxopentanoic Acid

Starting compound: example 24; 61% yield.

¹H NMR (300 MHz, DMSO-d₆) δ (TMS): 8.12 (d, J=7.3 Hz, 1H), 8.00 (s, 1H), 7.88 (s, 2H), 4.31 (m, 2H), 4.07 (m, 1H), 3.76 (broad d, J=13 Hz, 1H), 3.38 (m, 1H), 3.20 (m, 1H), 2.99 (m, 1H), 2.62 (m, 6H), 2.48 (m, 2H), 2.28 (m, 2H), 1.98 (m, 1H), 1.79 (m, 3H), 1.60 (m, 3H), 1.40 (m, 1H), 1.34 (m, 2H), 1.01 (m, 2H), 0.99 (t, J=7.1 Hz, 6H).

MS: m/z 619 (MH⁺).

### EXAMPLE 92

### (2S)-2-[1-(3,5-Dichlorophenylsulfonyl)-L-prolylamino]-5-oxo-5-[4-[2-(1-pyrrolidinyl)ethyl]piperidin-1-yl]pentanoic Acid

Starting compound: example 25; 64% yield.

MS: m/z 617 (MH⁺).

### EXAMPLE 93

### (2S)-2-[1-(3,5-Dichlorophenylsulfonyl)-L-prolylamino]-5-[4-[2-[(4-methylpiperazin-1-yl)carbonyloxy]ethyl]piperidin-1-yl]5-oxopentanoic Acid

Starting compound: example 26; 34% yield.

¹H NMR (300 MHz, CD₃OD) δ (TMS): 7.89 (s, 2H), 7.80 (s, 1H), 4.84 (s, 2H), 4.55 (m, 1H), 4.33 (m, 1H), 4.23 (m, 1H), 4.15 (m, 2H), 3.91 (m, 1H), 3.53 (m, 4H), 3.31 (m, 1H), 3.02 (m, 1H), 2.61 (m, 4H), 2.55 (m, 2H), 2.45 (s, 3H), 2.25 (m, 1H), 2.00 (m, 4H), 1.70 (m, 8H), 1.28 (m, 2H).

MS: m/z 690 (MH⁺).

### EXAMPLE 94

### (2S)-2-[1-(3,5-Dichlorophenylsulfonyl)-L-prolylamino]-5-oxo-5-[4-(1-piperidylmethyl)piperidin-1-yl]pentanoic Acid

Starting compound: example 30; 56% yield.

MS: m/z 617 (MH⁺).

### EXAMPLE 95

### (2S)-2-[1-(3,5-Dichlorophenylsulfonyl)-L-prolyl]amino-3-[[4 (dimethylaminomethyl)piperidin-1-ylcarbonyl]amino]propionic Acid

Starting compound: example 45; 51% yield.

¹H NMR (300 MHz, CD₃OD) δ (TMS): 7.90 (s, 2H), 7.81 (s, 1H), 4.91 (s, 3H), 4.57 (m, 1H), 4.40 (m, 1H), 4.20 (m, 1H), 4.02 (m, 2H) 3.67 (m, 1H), 3.50 (m, 2H), 3.30 (m, 1H), 2.94 (m, 1H), 2.81 (m, 2H), 2.79 (s, 6H), 2.05 (m, 4H), 1.70 (m, 3H), 1.22 (m, 2H).

MS: m/z 578 (MH⁺).

### EXAMPLE 96

### (2S)-2-[1-(3,5-Dichlorophenylsulfonyl)-L-prolylamino]-5-[4-[(ethylamino)methyl]piperidin-1-yl]-5-oxopentanoic Acid

Starting compound: example 31; 37% yield.

MS: m/z 577 (MH⁺).

### EXAMPLE 97

### (2S)-2-[1-(3,5-Dichlorophenylsulfonyl)-L-prolylamino]-5-[4-[[(4-methylpiperazin-1-yl)carbonylamino]methyl]piperidin-1-yl]-5-oxopentanoic Acid

Starting compound: example 32; 26% yield.

MS: m/z 675 (MH⁺).

### EXAMPLE 98: (2S)-2-[1-(3,5-Dichlorophenylsulfonyl)-L-prolyl]amino-3-[4-(dimethylaminomethyl)piperidin-1-ylcarbonyloxy]propionic Acid

Starting compound: example 47; 11% yield.

¹H NMR (300 MHz, CD₃OD) δ (TMS): 7.88 (s, 2H), 7.81 (s, 1H), 4.83 (s, 2H), 4.58 (m, 2H), 4.35 (m, 1H), 4.28 (m, 1H), 4.12 (m, 2H), 3.53 (m, 1H), 3.27 (m, 1H), 2.80 (m, 2H), 2.70 (m, 2H), 2.68 (s, 6H), 1.90 (m, 4H), 1.70 (m, 3H), 1.21 (m, 2H).

MS: m/z 577 (MH⁺).

### EXAMPLE 99

### (2S)-2-[1-(3,5-Dichlorophenylsulfonyl)-L-prolylamino]-5-oxo-5-[4-(4-pyridylaminomethyl)piperidin-1-yl]pentanoic Acid

To a solution of methyl (2S)-2-[1-(3,5-dichlorophenylsulfonyl)-L-prolylamino]-5-oxo-5-[4-(4-pyridylaminomethyl)piperidin-1-yl]pentanoate (obtained in example 33) (200 mg, 0.31 mmol) in THF (1 mL), LiOH.H₂O (26 mg, 0.62 mmol) dissolved in H₂O (2.6 mL) was added. The reaction mixture was stirred overnight at room temperature and THF was evaporated. The resulting residue was brought to neutral pH by the addition of 1 N HCl and the mixture was concentrated to a volume of 1 mL by evaporation of the solvent. Next, it was chromatographed using a Supelco Supelclean LC-18 column, using first H₂O and then MeOH as eluents. The fraction eluted with MeOH was concentrated to dryness, to give 86 mg of the title compound of the example (44% yield).

¹H NMR (300 MHz, CD₃OD) δ (TMS): 8.00 (d, J=6.5 Hz, 2H), 7.87 (s, 2H), 7.79 (s, 1H), 6.82 (d, J=6.5 Hz, 2H), 4.85 (s, 3H), 4.53 (m, 1H), 4.30 (m, 1H), 4.24 (m, 1H), 4.00 (m, 1H), 3.52 (m, 1H), 3.22 (m, 3H), 3.04 (m, 1H), 2.60 (m, 3H) 2.25 (m, 1H) 2.01 (m, 8H), 1.28 (m, 2H).

MS: m/z 626 (MH⁺).

Following a similar procedure to that described in example 99, but starting in each case from a suitable ester, the following compounds were obtained:

### EXAMPLE 100

### (2S)-5-[4-[2-[(4-Methylpiperazin-1-yl)carbonyloxy]ethyl]piperidin-1-yl]-5-oxo-2-[1-tosyl-L-prolylamino]pentanoic Acid

Starting compound: example 48; 16% yield.

¹H NMR (300 MHz, CD₃OD) δ (TMS): 7.80 (d, J=7.8 Hz, 2H), 7.44 (d, J=7.8 Hz, 2H), 4.87 (s, 2H), 4.47 (m, 1H), 4.26 (m, 1H), 4.15 (m, 3H), 3.98 (m, 1H), 3.47 (m, 4H), 3.31 (m, 1H), 3.02 (m, 1H), 2.61 (m, 4H), 2.55 (m, 2H), 2.45 (s, 3H), 2.29 (s, 3H), 2.25 (m, 1H), 2.00 (m, 4H), 1.70 (m, 8H), 1.28 (m, 2H).

MS: m/z 634 (MH⁺).

### EXAMPLE 101

### (2S)-2-[1-(3,5-Dichlorophenylsulfonyl)-L-prolylamino]-5-[4-[(4-methylpiperazin-1-yl)carbonyloxymethyl]piperidin-1-yl]-5-oxopentanoic Acid

Starting compound: example 50; 20% yield.

¹H NMR (300 MHz, CD₃OD) δ (TMS): 7.90 (s, 2H), 7.80 (s, 1H), 4.85 (s, 2H), 4.50 (m, 1H), 4.28 (m, 2H), 4.00 (m, 1H), 3.92 (m, 2H), 3.58 (m, 1H), 3.52 (m, 4H), 3.30 (m, 1H), 3.00 (m, 1H), 2.60 (m, 3H), 2.36 (m, 4H), 2.28 (s, 3H), 2.25 (m, 1H), 1.98 (m, 5H), 1.72 (m, 3H), 1.24 (m, 2H).

MS: m/z 673 (MH⁻).

### EXAMPLE 102

### (2S)-2-[1-(3,5-Dichlorophenylsulfonyl)-L-prolylamino]-5-oxo-5-[4-(4-pyridyloxymethyl)piperidin-1-yl]pentanoic Acid

Starting compound: example 51; 11% yield.

¹H NMR (300 MHz, CD₃OD) δ (TMS): 8.32 (m, 2H), 7.88 (s, 2H), 7.75 (s, 1H), 6.98 (m, 2H), 4.87 (s, 2H), 4.58 (m, 1H), 4.39 (m, 1H), 4.22 (m, 1H), 4.04 (m, 1H), 3.93 (d, J=6 Hz, 2H), 3.51 (m, 1H), 3.29 (m, 1H), 3.08 (m, 1H), 2.65 (m, 1H), 2.58 (m, 2H), 2.27 (m, 1H), 1.98 (m, 5H), 1.85 (m, 2H), 1.67 (m, 1H), 1.31 (m, 2H).

MS: m/z 625 (MH⁻).

### EXAMPLE 103

### (2S)-5-[4-(4-Methylpiperazin-1-ylmethyl)piperidin-1-yl]-5-oxo-2-[1-tosyl-L-prolylamino]pentanoic Acid

Starting compound: example 52; 42% yield.

MS: m/z 575 (MH⁻).

### EXAMPLE 104

### (2S)-5-[4-(Dimethylaminomethyl)piperidin-1-yl]-5-oxo-2-[1-tosyl-L-prolylamino]pentanoic Acid

Starting compound: example 53; 47% yield.

¹H NMR (300 MHz, CD₃OD) δ (TMS): 7.85 (d, J=8 Hz, 2H), 7.49 (d, J=8 Hz, 2H), 4.92 (s, 2H), 4.57 (m, 1H), 4.32 (m, 1H), 4.15 (m, 1H), 4.05 (m, 1H), 3.55 (m, 1H), 3.26 (m, 1H), 3.12 (m, 1H), 2.79 (d, J=7 Hz, 2H), 2.71 (s, 6H), 2.59 (m, 1H), 2.52 (m, 2H), 2.50 (s, 3H), 2.45 (m, 1H), 2.20 (m, 1H), 2.00 (m, 2H), 1.90 (m, 4H), 1.66 (m, 1H), 1.20 (m, 2H).

MS: m/z 521 (MH⁺).

### EXAMPLE 105

### (2S)-3-[[4-(1-Piperidylmethyl)piperidin-1-ylcarbonyl]amino]-2-[N-tosyl-L-prolyl]aminopropionic Acid

Starting compound: example 46; 41% yield.

¹H NMR (300 MHz, CD₃OD) δ (TMS): 7.8 (d, J=8 Hz, 2H), 7.43 (d, J=8 Hz, 2H), 4.89 (s, 3H), 4.31 (m, 1H), 4.11 (m, 1H), 4.03 (m, 2H), 3.69 (m, 1H), 3.48 (m, 2H), 3.24 (m, 1H), 2.82 (m, 4H), 2.66 (m, 2H), 2.44 (s, 3H), 2.00. (m, 2H), 1.87 (m, 8H), 1.71 (m, 4H), 1.24 (m, 3H).

MS: m/z 564 (MH⁺).

### EXAMPLE 106

### (2S)-2-[1-(3,5-Dichlorophenylsulfonyl)-L-prolylamino]-5-[4-[(N-ethyl-N-isobutoxycarbonylamino)methyl]piperidin-1-yl]-5-oxopentanoic Acid

To a solution of methyl (2S)-2-[1-(3,5-dichlorophenylsulfonyl)-L-prolylamino]-5-[4-[(N-ethyl-N-isobutoxycarbonylamino)methyl]piperidin-1-yl]-5-oxopentanoate (obtained in example 34) (159 mg, 0.23 mmol) in THF (1.5 mL), LiOH.H₂O (19 mg, 0.45 mmol) dissolved in H₂O (1.9 mL) was added. The reaction mixture was stirred overnight at room temperature and THF was evaporated. The resulting residue was cooled to 0° C. and acidified by adding 1 N HCl until reaching the maximal turbidity. The solid obtained was collected by filtration, it was dried and purified using a Supelco Supelclean LC-18 column, sequentially using H₂O/MeOH 5%, 0.2 M NaHCO₃, 2 M NaOH, 1 N HCl, H₂O and MeOH as eluents. The fraction eluted with MeOH was concentrated to dryness, to give 45 mg of the title compound of the example (29% yield).

¹H NMR (300 MHz, CD₃OD) δ (TMS): 7.88 (s, 2H), 7.80 (s, 1H), 4.85 (s, 2H), 4.49 (m, 2H), 4.26 (m, 1H), 4.01 (m, 1H), 3.81 (m, 2H), 3.52 (m, 1H), 3.27 (m, 2H), 3.12 (m, 1H), 3.01 (m, 1H), 2.60 (m, 3H), 2.25 (m, 1H), 2.00 (m, 5H), 1.65 (m, 4H), 1.32 (m, 2H), 1.18 (m, 5H), 0.92 (m, 6H).

MS: m/z 677 (MH⁺).

### EXAMPLE 107

### Methyl (2S)-2-[1-(3,5-dichlorophenylsulfonyl)-L-prolyl]amino-3-[[4-[2-[(4-methylpiperazin-1-yl)carbonyloxy]ethyl]piperidin-1-ylcarbonyl]amino]propionate

A solution of the compounds obtained in reference examples 57 (0.65 g, 1.44 mmol) and 35 (0.88 g, 3.4 mmol) and DIEA (0.88 mL, 5 mmol) in DMF (4 mL) was irradiated with a multimode microwaves at 140° C. for 1.5 min and at 150° C. for 40 min at a potency of 450 W. The resulting mixture was concentrated to dryness and partitioned three times between EtOAc and saturated NaHCO₃ solution. The combined organic phases were dried over sodium sulfate and concentrated, to afford a crude product which was purified by chromatography on silica gel, using a CH₂Cl₂/MeOH (9:1) mixture as eluent. 0.42 g of the title compound of the example was obtained (42% yield).

¹H NMR (300 MHz, CDCl₃) δ (TMS): 7.85 (m, 1H), 7.72 (s, 2H), 7.59 (s, 1H), 5.07 (m, 1H), 4.51 (m, 1H), 4.44 (m, 1H), 4.14 (m, 2H), 3.95 (m, 1H), 3.81 (s, 3H), 3.70 (m, 1H), 3.63 (m, 2H), 3.47 (m, 4H), 3.26 (m, 1H), 3.08 (m, 1H), 2.75 (m, 1H), 2.63 (m, 1H), 2.38 (m, 4H), 2.32 (s, 3H), 2.12 (m, 1H), 1.5-2.0 (m, 8H), 1.12 (m, 2H).

Following a similar procedure to that described in example 107, but using a suitable amine in each case, the following compounds were obtained:

### EXAMPLE 108

### Methyl (2S)-2-[1-(3,5-dichlorophenylsulfonyl)-L-prolyl]amino-3-[[4-[2-[(4-methylpiperidin-1-yl)carbonyloxy]ethyl]piperidin-1-ylcarbonyl]amino]propionate

Compounds used: reference examples 57 and 60; 51% yield.

¹H NMR (300 MHz, CD₃OD) δ (TMS): 7.87 (s, 2H), 7.78 (m, 1H), 4.80 (s, 2H), 4.43 (m, 1H), 4.12 (m, 1H), 3.9-4.10 (m, 6H), 3.71 (s, 3H), 3.51 (m, 1H), 3.49 (m, 2H), 3.26 (m, 1H), 2.74 (m, 4H), 1.95 (m, 4H), 1.45-1.75 (m, 8H), 1.20 (m, 4H), 0.95 (d, J=6.5 Hz, 3H).

### EXAMPLE 109

### Methyl (2S)-2-[1-(3,5-dichlorophenylsulfonyl)-L-prolyl]amino-3-[[4-[2-[[4-(ethoxycarbonyl)piperazin-1-yl]carbonyloxy]ethyl]piperidin-1-ylcarbonyl]amino]propionate

Compounds used: reference examples 57 and 59; 28% yield.

¹H NMR (300 MHz, CDCl₃) δ (TMS): 7.82 (m, 1H), 7.78 (s, 2H), 7.70 (s, 1H), 5.10 (m, 1H), 4.55 (m, 1H), 4.15 (m, 5H), 3.99 (m, 2H), 3.72 (s, 3H), 3.68 (m, 1H), 3.65(m, 2H), 3.48 (s, 8H), 3.18 (m, 1H), 2.70 (m, 2H), 2.17 (m, 1H), 1.5-2.0 (m, 8H), 1.25 (m, 5H).

### EXAMPLE 110

### Methyl (2S)-2-[1-(3,5-dichlorophenylsulfonyl)-L-prolyl]amino-3-[[4-[2-[[4-4-pyridyl)piperazin-1-yl]carbonyloxy]ethyl]piperidin-1-ylcarbonyl]amino]propionate

Compounds used: reference examples 57 and 58; 33% yield.

¹H NMR (300 MHz, CDCl₃) δ (TMS): 8.30 (d, J=5 Hz, 2H), 7.85 (m, 1H), 7.77 (s, 2H), 7.60 (s, 1H), 6.66 (d, J=5 Hz, 2H), 5.17 (m, 1H), 4.49 (m, 1H), 4.13 (m, 3H), 4.01 (m, 2H), 3.75 (s, 3H), 3.70 (m, 1H), 3.61 (m, 6H), 3.38 (m, 4H), 3.22 (m, 1H), 2.75 (m, 2H), 2.10 (m, 1H), 1.5-2.0 (m, 8H), 1.20 (m, 2H).

### EXAMPLE 111

### Methyl (2S)-2-[1-(3,5-dichlorophenylsulfonyl)-L-prolyl]amino-3-[[4-[2-[(cis-2,6-dimethylmorpholin-4-yl)carbonyloxy]ethyl]piperidin-1-ylcarbonyl]amino]propionate

Compounds used: reference examples 57 and 62; 23% yield.

¹H NMR (300 MHz, CD₃OD) δ (TMS): 7.87 (s, 1H), 7.80 (s, 2H), 4.86 (s, 2H), 4.43 (m, 1H), 4.11 (m, 3H), 3.97 (m, 2H), 3.85 (m, 2H), 3.71 (s, 3H), 3.63 (m, 2H), 3.51 (m, 5H), 3.27 (m, 1H), 2.74 (m, 2H), 2.49 (m, 2H), 1.95 (m, 3H), 1.67 (m, 2H), 1.56 (m, 2H), 1.10 (d, J=6 Hz, 6H), 1.20 (m, 2H).

### EXAMPLE 112

### (2S)-2-[1-(3,5-Dichlorophenylsulfonyl)-L-prolyl]amino-3-[[4-[2-[(4-methylhomopiperazin-1-yl)carbonyloxy]ethyl]piperidin-1-ylcarbonyl]amino]propionic Acid

Compounds used: reference examples 57 and 61; 10% yield.

¹H NMR (300 MHz, CD₃OD) δ (TMS): 7.88 (s, 2H), 7.79 (s, 1H), 4.88 (s, 3H), 4.30 (m, 1H), 4.11 (m, 3H), 4.01 (m, 2H), 3.60 (m, 5H), 3.55 (m, 2H), 3.20 (m, 1H), 2.88 (m, 4H), 2.73 (m, 2H), 2.50 (s, 3H), 1.8-2.0 (m, 5H), 1.5-1.7 (m, 6H), 1.20 (m, 2H).

MS: m/z 705 (MH⁺).

### EXAMPLE 113

### Methyl (2S)-2-[1-(3,5-dichlorophenylsulfonyl)-L-prolyl]amino-3-[[4-(2-ethylimidazo[4,5-c]pyridin-1-ylmethyl)piperidin-1-ylcarbonyl]amino]propionate

Following a similar procedure to that described in example 44, but using the compounds obtained in reference examples 23 and 0.52 as starting amines, the title compound of the example was obtained in 7% yield.

¹H NMR (300 MHz, CDCl₃) δ (TMS): 9.03 (s, 1H), 8.37 (d, J=5.4 Hz, 1H), 7.80 (s, 2H), 7.63 (s, 1H), 7.27 (m, 2H), 5.24 (m, 1H), 4.54 (m, 1H), 4.12 (m, 1H), 4.08 (m, 1H), 4.01 (d, J=7.2 Hz, 2H), 3.81 (m, 1H), 3.71 (double s, 3H), 3.67 (m, 2H), 3.25 (m, 2H), 2.91 (q, J=7.5 Hz, 2H), 2.72 (m, 2H), 2.21 (m, 1H), 2.02 (m, 2H), 1.5-1.9 (m, 4H), 1.54 (t, J=7.5 Hz, 3H), 1.28 (m, 2H).

### EXAMPLE 114

### Methyl (2S)-2-[1-(3,5-dichlorophenylsulfonyl)-L-prolylamino]-5-oxo-5-[4-[2-(4-phenylpiperazin-1-ylcarbonyloxy)ethyl]piperidin-1-yl]pentanoate

To a solution of the acid obtained in reference example 48 (0.72 g, 1.5 mmol) in DMF (10 mL), the amine obtained in reference example 56 (0.6 g, 1.5 mmol), HBTU (584 mg, 1.5 mmol) and DIEA (1.07 mL, 6 mmol) were added under argon atmosphere, and the reaction mixture was stirred overnight at room temperature. DMF was evaporated and the crude product obtained was partitioned three times between EtOAc and saturated NaHCO₃ solution. The combined organic phases were dried over sodium sulfate and concentrated, to afford a crude product which was purified by chromatography on silica gel, using EtOAc as eluent. The title compound of the example was obtained in quantitative yield.

¹H NMR (300 MHz, CDCl₃) δ (TMS): 7.77 (s, 2H), 7.71 (m, 1H), 7.58 (s, 1H), 7.28 (m, 2H), 6.90 (m, 3H), 4.55 (m, 1H), 4.47 (m, 1H), 4.15 (m, 4H), 3.80 (m, 1H), 3.76 (s, 3H), 3.57 (m, 5H), 3.25 (m, 1H), 3.16 (m, 4H), 3.00 (m, 1H), 2.55 (m, 2H), 2.16 (m, 2H). 1.7-1.9 (m, 4H), 1.5-1.6 (m, 5H), 1.15 (m, 2H).

### EXAMPLE 115

### Methyl (2S)-2-[1-(3,5-dichlorophenylsulfonyl)-L-prolylamino]-5-oxo-5-[4-(2-propylimidazo[4,5-c]pyridin-1-ylmethyl)piperidin-1-yl]pentanoate

Following a similar procedure to that described in example 114, but using the amine obtained in reference example 55 instead of the amine obtained in reference example 56, the title compound of the example was obtained in quantitative yield.

¹H NMR (300 MHz, CDCl₃) δ (TMS): 9.00 (s, 1H), 8.35 (d, J=4.2 Hz, 1H), 7.77 (m, 2H), 7.60 (m, 2H), 7.23 (m, 1H), 4.72 (m, 1H), 4.54 (m, 1H), 4.14 (m, 1H), 4.00 (m, 2H), 3.97 (m, 1H), 3.76 (s, 1.5H), 3.74 (s, 1.5H), 3.66 (m, 1H), 3.08 (m, 1H), 2.85 (m, 2H), 2.2-2.6 (m, 6H), 2.12 (m, 2H), 1.90 (m, 3H), 1.75(m, 2H), 1.61 (m, 2H), 1.3 (m, 2H), 1.05 (m, 3H).

### EXAMPLE 116

### (2S)-2-[1-(3,5-Dichlorophenylsulfonyl)-L-prolyl]amino-3-[[4-[2-[(4-methylpiperazin-1-yl)carbonyloxy]ethyl]piperidin-1-ylcarbonyl]amino]propionic Acid

Following a similar procedure to that described in example 99, but starting from the compound obtained in example 107 instead of the compound obtained in example 33, the title compound of the example was obtained in 61% yield.

¹H NMR (300 MHz, CD₃OD) δ (TMS): 7.88 (s, 2H), 7.78 (s, 1H), 4.84 (s, 3H), 4.34 (m, 1H), 4.16 (m, 3H), 3.95 (m, 2H), 3.45-3.70 (m, 7H), 3.26 (m, 1H), 3.08 (m, 1H), 2.65-2.85 (m, 6H), 2.62 (s, 3H), 1.5-2.0 (m, 8H), 1.12 (m, 2H).

MS: m/z 691 (MH⁺).

Following a similar procedure to that described in example 54, but starting in each case from a suitable ester, the following compounds were obtained:

### EXAMPLE 117

### (2S)-2-[1-(3,5-Dichlorophenylsulfonyl)-L-prolylamino]-5-oxo-5-[4-[2-(4-phenylpiperazin-1-ylcarbonyloxy)ethyl]piperidin-1-yl]pentanoic Acid

Starting compound: example 114; 54% yield.

MS: m/z 752 (MH⁺).

### EXAMPLE 118

### (2S)-2-[1-(3,5-Dichlorophenylsulfonyl)-L-prolyl]amino-3-[[4-[2-1(4-methylpiperidin-1-yl)carbonyloxy]ethyl]piperidin-1-ylcarbonyl]amino]propionic Acid

Starting compound: example 108; 59% yield.

¹H NMR (300 MHz, CD₃OD) δ (TMS): 7.88 (s, 2H), 7.78 (s, 1H), 4.80 (s, 3H), 4.44 (m, 1H), 4.15 (m, 1H), 3.9-4.00 (m, 6H), 3.66 (m, 1H), 3.55 (m, 2H), 3.30 (m, 1H), 2.77 (m, 4H), 2.00 (m, 4H), 1.5-1.75 (m, 8H), 1.20 (m, 4H), 0.92 (d, J=6.6 Hz, 3H).

MS: m/z 690 (MH⁺).

Following a similar procedure to that described in example 70, but starting in each case from a suitable ester, the following compounds were obtained:

### EXAMPLE 119

### (2S)-2-[1-(3,5-Dichlorophenylsulfonyl)-L-prolylamino]-5-oxo-5-[4-(2-propylimidazo[4,5-c]pyridin-1-ylmethyl)piperidin-1-yl]pentanoic Acid

Starting compound: example 115; 34% yield.

¹H NMR (300 MHz, CD₃OD) δ (TMS): 8.82 (s, 1H), 8.40 (s, 1H), 8.28 (d, J=4.8 Hz, 1H), 7.87 (s, 1H), 7.85 (s, 1H), 7.68 (m, 1H), 4.90 (s, 2H), 4.56 (m, 1H), 4.37 (m, 1H), 4.17 (m, 3H), 3.99 (m, 1H), 3.52 (m, 1H), 3.28 (m, 1H), 2.92 (m, 3H), 2.54 (m, 3H), 2.23 (m, 2H), 1.8-2.05 (m, 6H), 1.45-1.75 (m, 3H), 1.2-1.4 (m, 2H), 1.03 (m, 3H).

MS: m/z 693 (MH⁺).

### EXAMPLE 120

### (2S)-2-[1-(3,5-Dichlorophenylsulfonyl)-L-prolyl]amino-3-[[4-[2-[[4-(ethoxycarbonyl)piperazin-1-yl]carbonyloxy]ethyl]piperidin-1-ylcarbonyl]amino]propionic Acid

Starting compound: example 109; 27% yield.

¹H NMR (300 MHz, CDCl₃) δ (TMS): 7.78 (s, 2H), 7.83 (s, 1H), 4.86 (s, 3H), 4.37 (m, 1H), 4.15 (m, 5H), 4.00 (m, 2H), 3.69 (m, 1H), 3.55 (m, 2H), 3.41 (s, 8H), 3.26 (m, 1H), 2.72 (m, 2H), 2.05 (m, 3H), 1.5-1.8 (m, 6H), 1.23 (t, J=7.2 Hz, 3H), 1.20 (m, 2H).

MS: m/z 749 (MH⁺).

### EXAMPLE 121

### (2S)-2-[1-(3,5-Dichlorophenylsulfonyl)-L-prolyl]amino-3-[[4-[2-[[4-(4-pyridyl)piperazin-1-yl]carbonyloxy]ethyl]piperidin-1-ylcarbonyl]amino]propionic Acid

Starting compound: example 110; 69% yield.

¹H NMR (300 MHz, CD₃OD) δ (TMS): 8.11 (d, J=7.2 Hz, 2H), 7.87 (s, 2H), 7.76 (s, 1H), 7.01 (d, J=7.2 Hz, 2H), 4.86 (s, 3H), 4.35 (m, 1H), 4.15 (m, 3H), 4.00 (m, 2H), 3.65 (m, 1H), 3.61 (m, 4H), 3.55 (m, 2H), 3.20-3.30 (m, 5H), 2.73 (m, 2H), 1.8-2.0 (m, 3H), 1.5-1.7 (m, 6H), 1.20 (m, 2H).

MS: m/z 754 (MH⁺).

### EXAMPLE 122

### (2S)-2-[1-(3,5-Dichlorophenylsulfonyl)-L-prolyl]amino-3-[[4-[2-[(cis-2,6-dimethylmorpholin-4-yl)carbonyloxy]ethyl]piperidin-1-ylcarbonyl]amino]propionic Acid

Starting compound: example 111; 8% yield.

MS: m/z 754 (MH⁺).

### EXAMPLE 123

### Lithium (2S)-2-[1-(3,5-dichlorophenylsulfonyl)-L-prolyl]amino-3-[[4-(2-ethylimidazo[4,5-c]pyridin-1-ylmethyl)piperidin-1-ylcarbonyl]amino]propionate

To a solution of the compound obtained in example 113 (23 mg, 0.03 mmol) in THF (0.3 mL) and H₂O (0.15 mL), LiOH.H₂O (1.4 mg, 0.03 mmol) was added, and the resulting mixture was stirred overnight at room temperature. The resulting solution was concentrated to dryness, affording the title compound of the example in quantitative yield.

MS: m/z 680.4 (MH⁺).

1-37. (canceled)

38. A compound of general formula I: 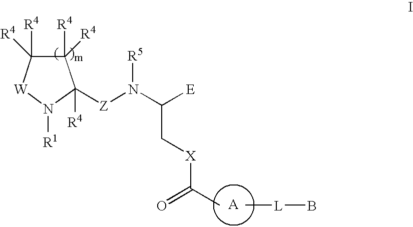
wherein:
R¹ represents —SO₂R², —COR² or —CH₂R³;
R² represents C₁₋₈ alkyl, C₂₋₈ alkenyl or C₂₋₈ alkynyl, which can be optionally substituted with one or more groups R^{a}, or R² represents Cy, CyC₁₋₄ alkyl, CyC₂₋₄ alkenyl or CyC₂₋₄ alkynyl, where the groups Cy can be optionally substituted with one or more groups R^{b};
R³ represents hydrogen, C₁₋₈ alkyl, C₂₋₈ alkenyl or C₂₋₈ alkynyl, where the groups C₁₋₈ alkyl, C₂₋₈ alkenyl and C₂₋₈ alkynyl can be optionally substituted with one or more groups R^{c}, or R³ represents Cy or CyC₁₋₄ alkyl, where the groups Cy can be optionally substituted with one or more groups selected from R^{c} and R^{d};
each R⁴ independently represents hydrogen, C₁₋₈ alkyl, Cy or CyC₁₋₄ alkyl, where the C₁₋₈ alkyl group can be optionally substituted with one or more groups R^{c} and where the groups Cy can be optionally substituted with one or more groups selected from R^{c} and R^{d};
W represents —CR⁴R⁴— when R¹ is —SO₂R² or —COR², or W represents —CO— when R¹ is —CH₂R³;
Z represents —CO— or —CS—;
E represents —COOR⁶, —CONR⁷R⁸ or 5-tetrazolyl;
X represents —CH₂—, —NR⁵— or —O—;
each R⁵ independently represents hydrogen or C₁₋₄ alkyl;
R⁶ represents hydrogen, C₁₋₈ alkyl, C₃₋₇ cycloalkyl or aryl, where the C₁₋₈ alkyl group can be optionally substituted with a group selected from C₃₋₇ cycloalkyl, aryl, —OR^{g}, —OCOR^{d}, —OCOOR^{d}, —COOR^{g} and —NHCOR^{g} and the aryl groups can be optionally substituted with one or more groups R^{b};
R⁷ represents hydrogen, C₁₋₈ alkyl, C₃₋₇ cycloalkyl, aryl or —SO₂R^{d}, where the C₁₋₈ alkyl group can be optionally substituted with a group selected from C₃₋₇ cycloalkyl, aryl, —SO₂R^{d}, —COOR^{g} and —COR^{d};
R⁸ represents hydrogen or C₁₋₈ alkyl;
or R⁷ and R⁸ together with the nitrogen atom to which they are bound can form a Het¹;
A represents C₃₋₇ cycloalkyl or Het¹, which can be optionally substituted with one or more groups selected from oxo, C₁₋₈ alkyl and C₁₋₈ haloalkyl;
L represents —(CR⁹R⁹)ₙ—;
each R⁹ independently represents hydrogen or C₁₋₄ alkyl;
B represents:
i) C₃₋₇ cycloalkyl, Het¹ or Het², which can be optionally substituted with one or more groups selected from oxo, R^{b} and Cy optionally substituted with one or more groups R^{b}; or
ii) a group selected from —COR^{e}, —NR^{f}R^{f}, —OR^{f}, —SR^{f}, S(O)ₚR^{e}, —CONR^{f}R^{f}, —NR^{f}COR^{f}, —NR^{f}CONR^{f}R^{f}, —NR^{f}CSNR^{f}R^{f}, —NR^{f}COOR^{e}, —OCOR^{e}, —OCONR^{f}R^{f}, —NR^{f}SO₂R^{e} and —SO₂NR^{f}R^{f};
m represents 0 or 1;
n represents 1, 2, 3 or 4;
p represents 1 or 2;
each R^{a} independently represents halogen, —COR^{d}, —OR^{g}, —NR^{g}R^{g}, —COOR^{g}, —OCOR^{d}, —CONR^{g}R^{g}, —NR^{g}COR^{g}, —OCONR^{g}R^{g} or —NR^{g}COOR^{d};
each R^{b} independently represents a group R^{a}, —NO₂, —SR^{g}, —S(O)ₚR^{d} or C₁₋₈ alkyl optionally substituted with one or more groups R^{c};
each R^{c} independently represents halogen, —OR^{h} or —NR^{h}R^{h};
each R^{d} independently represents C₁₋₈ alkyl, C₃₋₇ cycloalkyl or aryl, which can be optionally substituted with one or more groups R^{c};
each R^{e} independently represents C₁₋₈ alkyl, C₂₋₈ alkenyl or C₂₋₈ alkynyl, which can be optionally substituted with one or more groups R^{a}, or R^{e} represents Cy or CyC₁₋₄ alkyl, where the groups Cy optionally can be substituted with one or more groups selected from oxo, Cy* and R^{b}, and where the groups Cy* can be optionally substituted with one or more groups selected from oxo and R^{b};
each R^{f} independently represents hydrogen or any of the meanings described for R^{e};
or two groups R^{f} placed on the same nitrogen atom can be attached to each other to form together with said nitrogen atom a Het¹ which optionally can be substituted with one or more groups selected from oxo, Cy and R^{b}, where the groups Cy can be optionally substituted with one or more groups selected from oxo and R^{b};
each R^{g} independently represents hydrogen or any of the meanings described for R^{d};
or two groups R^{g} placed on the same nitrogen atom can be attached to each other to form together with said nitrogen atom Het¹ which optionally can be substituted with one or more groups selected from oxo, Cy and R^{b}, where the groups Cy optionally can be substituted with one or more groups selected from oxo and R^{b};
each R^{h} independently represents hydrogen, C₁₋₈ alkyl, C₃₋₇ cycloalkyl or aryl, where the groups C₁₋₈ alkyl, C₃₋₇ cycloalkyl and aryl optionally can be substituted with one or more halogen atoms;
Cy and Cy* independently represent aryl, C₃₋₇ cycloalkyl, Het¹ or Het²;
aryl in the above definitions represents phenyl or naphthyl;
Het¹ in the above definitions represents a saturated or unsaturated non-aromatic 5- to 7-membered monocyclic ring containing from one to four heteroatoms selected from N, O and S, which optionally can be fused to a phenyl, naphthyl or Het² ring, and which is chemically stable and obtainable through chemical synthesis; and
Het² in the above definitions represents an aromatic 5- to 7-membered monocyclic or 9- to 11-membered bicyclic ring, which contains from one to four heteroatoms selected from N, O and S, and which is chemically stable and obtainable through chemical synthesis;
or a salt, solvate or prodrug thereof.

39. A compound according to claim 38 wherein R¹ represents —SO₂R².

40. A compound according to claim 38 or 39 wherein R² represents aryl optionally substituted with one or more groups R^{b}.

41. A compound according to claim 38 wherein all the groups R⁴ represent hydrogen.

42. A compound according to claim 38 wherein R⁵ represents hydrogen.

43. A compound according to claim 38 wherein W represents —CR⁴R⁴—.

44. A compound according to claim 43 wherein W represents —CH₂—.

45. A compound according to claim 38 wherein Z represents —CO—.

46. A compound according to claim 38 wherein E represents —COOR⁶.

47. A compound according to claim 46 wherein E represents —COOH.

48. A compound according to claim 38 wherein m represents 1.

49. A compound according to claim 38 wherein X represents —NH—.

50. A compound according to claim 38 wherein X represents —CH₂—.

51. A compound according to claim 38 wherein X represents —O—.

52. A compound according to claim 38 wherein A represents piperidine or piperazine.

53. A compound according to claim 38 wherein L represents —(CH₂)ₙ—.

54. A compound according to claim 53 wherein L represents methylene or ethylene.

55. A compound according to claim 38 wherein B represents Het¹ or Het² optionally substituted with one or more groups selected from oxo, R^{b} and Cy optionally substituted with one or more groups R^{b}.

56. A compound according to claim 55 wherein B represents imidazopyridine optionally substituted with one or more groups selected from oxo, R^{b} and Cy optionally substituted with one or more groups R^{b}.

57. A compound according to claim 38 wherein B represents —NR^{f}R^{f}, —OR^{f}, —NR^{f}COR^{f}, —NR^{f}CONR^{f}R^{f}, —NR^{f}CSNR^{f}R, —NR^{f}COOR^{e} or —OCONR^{f}R^{f}.

58. A compound according to claim 57 wherein B represents —OCONR^{f}R^{f}.

59. A compound according to claim 58 wherein both groups R^{f} are attached to each other to form together with the nitrogen atom a Het¹, which optionally can be substituted with one or more groups selected from oxo, Cy and R^{b}, wherein the groups Cy optionally can be substituted with one or more groups selected from oxo and R^{b}.

60. A compound according to claim 38 selected from:
methyl (2S)-2-[1-(3,5-dichlorophenylsulfonyl)-L-prolylamino]-5-oxo-5-[4-(2-oxooxazolidin-3-ylmethyl)piperidin-1-yl]pentanoate;
methyl (2S)-2-[1-(3,5-dichlorophenylsulfonyl)-L-prolylamino]-5-[4-(2-oxopyrrolidin-1-ylmethyl)piperidin-1-yl]-5-oxopentanoate;
methyl (2S)-2-[1-(3,5-dichlorophenylsulfonyl)-L-prolylamino]-5-oxo-5-[4-(1-oxoisoindolin-2-ylmethyl)piperidin-1-yl]pentanoate;
methyl (2S)-2-[1-(3,5-dichlorophenylsulfonyl)-L-prolylamino]-5-[4-(2-ethyl-5,7-dimethylimidazo[4,5-b]pyridin-3-ylmethyl)piperidin-1-yl]-5-oxopentanoate;
methyl (2S)-5-[4-(2-methylimidazo[4,5-c]pyridin-1-yl methyl)piperidin-1-yl]-5-oxo-2-[1-tosyl-L-prolylamino]pentanoate;
methyl (2S)-5-oxo-5-[4-[(2-oxopyrrolidin-1-yl)methyl]piperidin-1-yl]-2-[1-tosyl-L-prolylamino]pentanoate;
methyl (2S)-5-oxo-5-[4-(2-phenylimidazol-1-ylmethyl)piperidin-1-yl]-2-[1-tosyl-L-prolylamino]pentanoate;
methyl (2S)-5-[4-[[1-(2-ethoxyethyl)benzimidazol-2-yl]methyl]piperazin-1-yl]-5-oxo-2-[[1-tosyl-L-prolyl]amino]pentanoate;
methyl (2S)-5-oxo-5-[4-(2-pyridylmethyl)piperazin-1-yl]-2-[[1-tosyl-L-prolyl]amino]pentanoate;
methyl (2S)-5-oxo-5-[4-(1-oxoisoindolin-2-ylmethyl)piperidin-1-yl]-2-[[1-tosyl-L-prolyl]amino]pentanoate;
methyl (2S)-5-oxo-5-[4-(2-thienylmethyl)piperazin-1-yl]-2-[[1-tosyl-L-prolyl]amino]pentanoate;
methyl (2S)-5-[4-[(2,5-dioxopyrrolidin-1-yl)methyl]piperidin-1-yl]-5-oxo-2-[[1-tosyl-L-prolyl]amino]pentanoate;
methyl (2S)-2-[1-(3,5-dichlorophenylsulfonyl)-L-prolylamino]-5-[4-[(3-methylbutanoylamino)methyl]piperidin-1-yl]-5-oxopentanoate;
methyl (2S)-5-[4-[(N′-tert-butylureido)methyl]piperidin-1-yl]-2-[1-(3,5-dichlorophenylsulfonyl)-L-prolylamino]-5-oxopentanoate;
methyl (2S)-2-[1-(3,5-dichlorophenylsulfonyl)-L-prolylamino]-5-[4-[(isobutoxycarbonylamino)methyl]piperidin-1-yl]-5-oxopentanoate;
methyl (2S)-5-oxo-2-[1-tosyl-L-prolyl]amino-5-[4-[[4-(trifluoromethyl)pyrimidin-2-yl]aminomethyl]piperidin-1-yl]pentanoate;
methyl (2S)-2-[1-(3,5-dichlorophenylsulfonyl)-L-prolylamino]-5-[4-[(isopropylsulfonylamino)methyl]piperidin-1-yl]-5-oxopentanoate;
methyl (2S)-2-[1-(3,5-dichlorophenylsulfonyl)-L-prolylamino]-5-[4-[(N′-isopropylthioureido)methyl]piperidin-1-yl]-5-oxopentanoate;
methyl (2S)-2-[1-(3,5-dichlorophenylsulfonyl)-L-prolylamino]-5-[4-[2-(2-ethyl-5,7-dimethylimidazo[4,5-b]pyridin-3-yl)ethyl]piperidin-1-yl]-5-oxopentanoate;
methyl (2S)-2-[1-(3,5-dichlorophenylsulfonyl)-L-prolylamino]-5-oxo-5-[4-[2-(pyrrolidin-1-ylcarbonyloxy)ethyl]piperidin-1-yl]pentanoate;
methyl (2S)-2-[1-(3,5-dichlorophenylsulfonyl)-L-prolylamino]-5-[4-(4-methyl piperazin-1-ylmethyl)piperidin-1-yl]-5-oxopentanoate;
methyl (2S)-2-[1-(3,5-dichlorophenylsulfonyl)-L-prolylamino]-5-[4-[2-(4-morpholinyl)ethyl]piperidin-1-yl]-5-oxopentanoate;
methyl (2S)-2-[1-(3,5-dichlorophenylsulfonyl)-L-prolylamino]-5-[4-[[(dimethylaminoacetyl)amino]methyl]piperidin-1-yl]-5-oxopentanoate;
methyl (2S)-2-[1-(3,5-dichlorophenylsulfonyl)-L-prolylamino]-5-[4-[2-(diethylamino)ethyl]piperidin-1-yl]-5-oxopentanoate;
methyl (2S)-2-[1-(3,5-dichlorophenylsulfonyl)-L-prolylamino]-5-oxo-5-[4-[2-(1-pyrrolidinyl)ethyl]piperidin-1-yl]pentanoate;
methyl (2S)-2-[1-(3,5-dichlorophenylsulfonyl)-L-prolylamino]-5-[4-[2-[(4-methyl piperazin-1-yl)carbonyloxy]ethyl]piperidin-1-yl]-5-oxopentanoate;
methyl (2S)-2-[1-(3,5-dichlorophenylsulfonyl)-L-prolylamino]-5-[4-[2-[(2-methoxyethyl)aminocarbonyloxy]ethyl]piperidin-1-yl]-5-oxopentanoate;
methyl (2S)-2-[1-(3,5-dichlorophenylsulfonyl)-L-prolylamino]-5-[4-(4-morpholinylcarbonylaminomethyl)piperidin-1-yl]-5-oxopentanoate;
methyl (2S)-2-[1-(3,5-dichlorophenylsulfonyl)-L-prolylamino]-5-oxo-5-[4-(1-pyrrolidinylcarbonylaminomethyl)piperidin-1-yl]pentanoate;
methyl (2S)-2-[1-(3,5-dichlorophenylsulfonyl)-L-prolylamino]-5-oxo-5-[4-(1-piperidylmethyl)piperidin-1-yl]pentanoate;
methyl (2S)-2-[1-(3,5-dichlorophenylsulfonyl)-L-prolylamino]-5-[4-[[N-ethyl-N-(trifluoroacetyl)amino]methyl]piperidin-1-yl]-5-oxopentanoate;
methyl (2S)-2-[1-(3,5-dichlorophenylsulfonyl)-L-prolylamino]-5-[4-[[(4-methylpiperazin-1-yl)carbonylamino]methyl]piperidin-1-yl]-5-oxopentanoate;
methyl (2S)-2-[1-(3,5-dichlorophenylsulfonyl)-L-prolylamino]-5-oxo-5-[4-(4-pyridylaminomethyl)piperidin-1-yl]pentanoate;
methyl (2S)-2-[1-(3,5-dichlorophenylsulfonyl)-L-prolylamino]-5-[4-[(N-ethyl-N-isobutoxycarbonylamino)methyl]piperidin-1-yl]-5-oxopentanoate;
methyl (2S)-2-[1-(3,5-dichlorophenylsulfonyl)-L-prolylamino]-5-[4-(2-methylimidazo[4,5-c]pyridin-1-ylmethyl)piperidin-1-yl]-5-oxopentanoate;
methyl (2S)-2-[1-(3,5-dichlorophenylsulfonyl)-L-prolylamino]-5-oxo-5-[4-(1-pyrrolylmethyl)piperidin-1-yl]pentanoate;
methyl (2S)-2-[1-(3,5-dichlorophenylsulfonyl)-L-prolylamino]-5-[4-(2,5-dimethylpyrrol-1-ylmethyl)piperidin-1-yl]-5-oxopentanoate;
methyl (2S)-2-[1-(3,5-dichlorophenylsulfonyl)-L-prolylamino]-5-[4-(dimethylaminomethyl)piperidin-1-yl]-5-oxopentanoate;
methyl (2S)-2-[1-(3,5-dichlorophenylsulfonyl)-L-prolylamino]-5-[4-[2-(dimethylamino)ethyl]piperazin-1-yl]-5-oxopentanoate;
methyl (2S)-2-[1-(3,5-dichlorophenylsulfonyl)-L-prolylamino]-5-[4-(2-ethylimidazo[4,5-c]pyridin-1-ylmethyl)piperidin-1-yl]-5-oxopentanoate;
methyl (2S)-2-[1-(3,5-dichlorophenylsulfonyl)-L-prolylamino]-5-oxo-5-[4-(2-oxo-2,3-dihydroimidazo[4,5-c]pyridin-1-ylmethyl)piperidin-1-yl]pentanoate;
methyl (2S)-2-[1-(3,5-dichlorophenylsulfonyl)-L-prolylamino]-5-[4-(2-isopropylaminoimidazo[4,5-c]pyridin-1-ylmethyl)piperidin-1-yl]-5-oxopentanoate;
methyl (2S)-2-[1-(3,5-dichlorophenylsulfonyl)-L-prolylamino]-5-[4-(diethylaminomethyl)piperidin-1-yl]-5-oxopentanoate;
methyl (2S)-2-[1-(3,5-dichlorophenylsulfonyl)-L-prolyl]amino-3-[[4-(2-methylimidazo[4,5-c]pyridin-1-ylmethyl)piperidin-1-ylcarbonyl]amino]propionate;
methyl (2S)-2-[1-(3,5-dichlorophenylsulfonyl)-L-prolyl]amino-3-[[4-(dimethylaminomethyl)piperidin-1-ylcarbonyl]amino]propionate;
methyl (2S)-3-[[4-(1-piperidylmethyl)piperidin-1-ylcarbonyl]amino]-2-[N-tosyl-L-prolyl]aminopropionate;
methyl (2S)-2-[1-(3,5-dichlorophenylsulfonyl)-L-prolyl]amino-3-[4-(dimethylaminomethyl)piperidin-1-ylcarbonyloxy]propionate;
methyl (2S)-5-[4-[2-[(4-methyl piperazin-1-yl)carbonyloxy]ethyl]piperidin-1-yl]-5-oxo-2-[1-tosyl-L-prolylamino]pentanoate;
methyl (2S)-2-[1-(3,5-dichlorophenylsulfonyl)-L-prolyl]amino-5-[4-[(diethylaminocarbonyloxy)methyl]piperidin-1-yl]-5-oxopentanoate;
methyl (2S)-2-[1-(3,5-dichlorophenylsulfonyl)-L-prolylamino]-5-[4-[(4-methylpiperazin-1-yl)carbonyloxymethyl]piperidin-1-yl]-5-oxopentanoate;
methyl (2S)-2-[1-(3,5-dichlorophenylsulfonyl)-L-prolylamino]-5-oxo-5-[4-(4-pyridyloxymethyl)piperidin-1-yl]pentanoate;
methyl (2S)-5-[4-(4-methyl piperazin-1-yl methyl)piperidin-1-yl]-5-oxo-2-[1-tosyl-L-prolylamino]pentanoate;
methyl (2S)-5-[4-(dimethylaminomethyl)piperidin-1-yl]-5-oxo-2-[1-tosyl-L-prolylamino]pentanoate;
(2S)-5-[4-[(1-oxoisoindolin-2-yl)methyl]piperidin-1-yl]-2-[1-(3,5-dichlorophenylsulfonyl)-L-prolylamino]-5-oxopentanoic acid;
(2S)-2-[1-(3,5-dichlorophenylsulfonyl)-L-prolylamino]-5-[4-[(3-methylbutanoylamino)methyl]piperidin-1-yl]-5-oxopentanoic acid;
(2S)-5-[4-[(N′-tert-butylureido)methyl]piperidin-1-yl]-2-[1-(3,5-dichlorophenylsulfonyl)-L-prolylamino]-5-oxopentanoic acid;
(2S)-2-[1-(3,5-dichlorophenylsulfonyl)-L-prolylamino]-5-[4-[(isobutoxycarbonylamino)methyl]piperidin-1-yl]-5-oxopentanoic acid;
(2S)-2-[1-(3,5-dichlorophenylsulfonyl)-L-prolylamino]-5-[4-[(N′-isopropylthioureido)methyl]piperidin-1-yl]-5-oxopentanoic acid;
(2S)-2-[1-(3,5-dichlorophenylsulfonyl)-L-prolylamino]-5-[4-[2-(2-ethyl-5,7-dimethylimidazo[4,5-b]pyridin-3-yl)ethyl]piperidin-1-yl]-5-oxopentanoic acid;
(2S)-2-[1-(3,5-dichlorophenylsulfonyl)-L-prolylamino]-5-[4-[(isopropylsulfonylamino)methyl]piperidin-1-yl]-5-oxopentanoic acid;
(2S)-2-[1-(3,5-dichlorophenylsulfonyl)-L-prolylamino]-5-oxo-5-[4-[2-(pyrrolidin-1-ylcarbonyloxy)ethyl]piperidin-1-yl]pentanoic acid;
(2S)-5-oxo-5-[4-[(2-oxopyrrolidin-1-yl)methyl]piperidin-1-yl]-2-[1-tosyl-L-prolylamino]pentanoic acid;
(2S)-5-oxo-5-[4-(2-phenylimidazol-1-ylmethyl)piperidin-1-yl]-2-[1-tosyl-L-prolylamino]pentanoic acid;
(2S)-5-oxo-2-[1-tosyl-L-prolyl]amino-5-[4-[[4-(trifluoromethyl)pyrimidin-2-yl]aminomethyl]piperidin-1-yl]pentanoic acid;
(2S)-2-[1-(3,5-dichlorophenylsulfonyl)-L-prolylamino]-5-[4-(2-ethyl-5,7-dimethylimidazo[4,5-b]pyridin-3-ylmethyl)piperidin-1-yl]-5-oxopentanoic acid;
(2S)-2-[1-(3,5-dichlorophenylsulfonyl)-L-prolylamino]-5-[4-[2-[(2-methoxyethyl)aminocarbonyloxy]ethyl]piperidin-1-yl]-5-oxopentanoic acid;
(2S)-2-[1-(3,5-dichlorophenylsulfonyl)-L-prolylamino]-5-[4-(4-morpholinylcarbonylaminomethyl)piperidin-1-yl]-5-oxopentanoic acid;
(2S)-2-[1-(3,5-dichlorophenylsulfonyl)-L-prolylamino]-5-oxo-5-[4-(1-pyrrolidinylcarbonylaminomethyl)piperidin-1-yl]pentanoic acid;
(2S)-2-[1-(3,5-dichlorophenylsulfonyl)-L-prolyl]amino-5-[4-[(diethylaminocarbonyloxy)methyl]piperidin-1-yl]-5-oxopentanoic acid;
(2S)-2-[1-(3,5-dichlorophenylsulfonyl)-L-prolylamino]-5-oxo-5-[4-(2-oxooxazolidin-3-ylmethyl)piperidin-1-yl]pentanoic acid;
(2S)-2-[1-(3,5-dichlorophenylsulfonyl)-L-prolylamino]-5-[4-(2-oxopyrrolidin-1-ylmethyl)piperidin-1-yl]-5-oxopentanoic acid;
(2S)-2-[1-(3,5-dichlorophenylsulfonyl)-L-prolylamino]-5-[4-(2-methylimidazo[4,5-c]pyridin-1-ylmethyl)piperidin-1-yl]-5-oxopentanoic acid;
(2S)-5-[4-(2-methylimidazo[4,5-c]pyridin-1-ylmethyl)piperidin-1-yl]-5-oxo-2-[1-tosyl-L-prolylamino]pentanoic acid;
(2S)-5-[4-[[1-(2-ethoxyethyl)benzimidazol-2-yl]methyl]piperazin-1-yl]-5-oxo-2-[[1-tosyl-L-prolyl]amino]pentanoic acid;
(2S)-5-oxo-5-[4-(2-pyridylmethyl)piperazin-1-yl]-2-[[1-tosyl-L-prolyl]amino]pentanoic acid;
(2S)-5-oxo-5-[4-(1-oxoisoindolin-2-yl methyl)piperidin-1-yl]-2-[[1-tosyl-L-prolyl]amino]pentanoic acid;
(2S)-5-oxo-5-[4-(2-thienylmethyl)piperazin-1-yl]-2-[[1-tosyl-L-prolyl]amino]pentanoic acid;
(2S)-5-[4-[(3-carboxypropionylamino)methyl]piperidin-1-yl]-5-oxo-2-[[1-tosyl-L-prolyl]amino]pentanoic acid;
(2S)-2-[1-(3,5-dichlorophenylsulfonyl)-L-prolylamino]-5-[4-(4-methylpiperazin-1-ylmethyl)piperidin-1-yl]-5-oxopentanoic acid;
(2S)-2-[1-(3,5-dichlorophenylsulfonyl)-L-prolylamino]-5-[4-[2-(4-morpholinyl)ethyl]piperidin-1-yl]-5-oxopentanoic acid;
(2S)-2-[1-(3,5-dichlorophenylsulfonyl)-L-prolylamino]-5-oxo-5-[4-(1-pyrrolylmethyl)piperidin-1-yl]pentanoic acid;
(2S)-2-[1-(3,5-dichlorophenylsulfonyl)-L-prolylamino]-5-[4-(2,5-dimethylpyrrol-1-ylmethyl)piperidin-1-yl]-5-oxopentanoic acid;
(2S)-2-[1-(3,5-dichlorophenylsulfonyl)-L-prolylamino]-5-[4-(dimethylaminomethyl)piperidin-1-yl]-5-oxopentanoic acid;
(2S)-2-[1-(3,5-dichlorophenylsulfonyl)-L-prolylamino]-5-[4-[2-(dimethylamino)ethyl]piperazin-1-yl]-5-oxopentanoic acid;
(2S)-2-[1-(3,5-dichlorophenylsulfonyl)-L-prolylamino]-5-[4-(2-ethylimidazo[4,5-c]pyridin-1-ylmethyl)piperidin-1-yl]-5-oxopentanoic acid;
(2S)-2-[1-(3,5-dichlorophenylsulfonyl)-L-prolylamino]-5-oxo-5-[4-(2-oxo-2,3-dihydroimidazo[4,5-c]pyridin-1-ylmethyl)piperidin-1-yl]pentanoic acid;
(2S)-2-[1-(3,5-dichlorophenylsulfonyl)-L-prolylamino]-5-[4-(2-isopropylaminoimidazo[4,5-c]pyridin-1-ylmethyl)piperidin-1-yl]-5-oxopentanoic acid;
(2S)-2-[1-(3,5-dichlorophenylsulfonyl)-L-prolylamino]-5-[4-(diethylaminomethyl)piperidin-1-yl]-5-oxopentanoic acid;
(2S)-2-[1-(3,5-dichlorophenylsulfonyl)-L-prolyl]amino-3-[[4-(2-methylimidazo[4,5-c]pyridin-1-ylmethyl)piperidin-1-ylcarbonyl]amino]propionic acid;
(2S)-2-[1-(3,5-dichlorophenylsulfonyl)-L-prolylamino]-5-[4-[[(dimethylaminoacetyl)amino]methyl]piperidin-1-yl]-5-oxopentanoic acid;
(2S)-2-[1-(3,5-dichlorophenylsulfonyl)-L-prolylamino]-5-[4-[2-(diethylamino)ethyl]piperidin-1-yl]-5-oxopentanoic acid;
(2S)-2-[1-(3,5-dichlorophenylsulfonyl)-L-prolylamino]-5-oxo-5-[4-[2-(1-pyrrolidinyl)ethyl]piperidin-1-yl]pentanoic acid;
(2S)-2-[1-(3,5-dichlorophenylsulfonyl)-L-prolylamino]-5-[4-[2-[(4-methylpiperazin-1-yl)carbonyloxy]ethyl]piperidin-1-yl]-5-oxopentanoic acid;
(2S)-2-[1-(3,5-dichlorophenylsulfonyl)-L-prolylamino]-5-oxo-5-[4-(1-piperidylmethyl)piperidin-1-yl]pentanoic acid;
(2S)-2-[1-(3,5-dichlorophenylsulfonyl)-L-prolyl]amino-3-[[4-(dimethylaminomethyl)piperidin-1-ylcarbonyl]amino]propionic acid;
(2S)-2-[1-(3,5-dichlorophenylsulfonyl)-L-prolylamino]-5-[4-[(ethylamino)methyl]piperidin-1-yl]-5-oxopentanoic acid;
(2S)-2-[1-(3,5-dichlorophenylsulfonyl)-L-prolylamino]-5-[4-[[(4-methylpiperazin-1-yl)carbonylamino]methyl]piperidin-1-yl]-5-oxopentanoic acid;
(2S)-2-[1-(3,5-dichlorophenylsulfonyl)-L-prolyl]amino-3-[4-(dimethylaminomethyl)piperidin-1-ylcarbonyloxy]propionic acid;
(2S)-2-[1-(3,5-dichlorophenylsulfonyl)-L-prolylamino]-5-oxo-5-[4-(4-pyridylaminomethyl)piperidin-1-yl]pentanoic acid;
(2S)-5-[4-[2-[(4-methyl piperazin-1-yl)carbonyloxy]ethyl]piperidin-1-yl]-5-oxo-2-[1-tosyl-L-prolylamino]pentanoic acid;
(2S)-2-[1-(3,5-dichlorophenylsulfonyl)-L-prolylamino]-5-[4-[(4-methylpiperazin-1-yl)carbonyloxymethyl]piperidin-1-yl]-5-oxopentanoic acid;
(2S)-2-[1-(3,5-dichlorophenylsulfonyl)-L-prolylamino]-5-oxo-5-[4-(4-pyridyloxymethyl)piperidin-1-yl]pentanoic acid;
(2S)-5-[4-(4-methylpiperazin-1-ylmethyl)piperidin-1-yl]-5-oxo-2-[1-tosyl-L-prolylamino]pentanoic acid;
(2S)-5-[4-(dimethylaminomethyl)piperidin-1-yl]-5-oxo-2-[1-tosyl-L-prolylamino]pentanoic acid;
(2S)-3-[[4-(1-piperidylmethyl)piperidin-1-ylcarbonyl]amino]-2-[N-tosyl-L-prolyl]aminopropionic acid;
(2S)-2-[1-(3,5-dichlorophenylsulfonyl)-L-prolylamino]-5-[4-[(N-ethyl-N-isobutoxycarbonylamino)methyl]piperidin-1-yl]-5-oxopentanoic acid;
methyl (2S)-2-[1-(3,5-dichlorophenylsulfonyl)-L-prolyl]amino-3-[[4-[2-[(4-methylpiperazin-1-yl)carbonyloxy]ethyl]piperidin-1-ylcarbonyl]amino]propionate;
methyl (2S)-2-[1-(3,5-dichlorophenylsulfonyl)-L-prolyl]amino-3-[[4-[2-[(4-methylpiperidin-1-yl)carbonyloxy]ethyl]piperidin-1-ylcarbonyl]amino]propionate;
methyl (2S)-2-[1-(3,5-dichlorophenylsulfonyl)-L-prolyl]amino-3-[[4-[2-[[4-(ethoxycarbonyl)piperazin-1-yl]carbonyloxy]ethyl]piperidin-1-ylcarbonyl]amino]propionate;
methyl (2S)-2-[1-(3,5-dichlorophenylsulfonyl)-L-prolyl]amino-3-[[4-[2-[[4-(4-pyridyl)piperazin-1-yl]carbonyloxy]ethyl]piperidin-1-ylcarbonyl]amino]propionate;
methyl (2S)-2-[1-(3,5-dichlorophenylsulfonyl)-L-prolyl]amino-3-[[4-[2-[(cis-2,6-dimethylmorpholin-4-yl)carbonyloxy]ethyl]piperidin-1-ylcarbonyl]amino]propionate;
(2S)-2-[1-(3,5-dichlorophenylsulfonyl)-L-prolyl]amino-3-[[4-[2-[(4-methylhomopiperazin-1-yl)carbonyloxy]ethyl]piperidin-1-ylcarbonyl]amino]propionic acid;
methyl (2S)-2-[1-(3,5-dichlorophenylsulfonyl)-L-prolyl]amino-3-[[4-(2-ethylimidazo[4,5-c]pyridin-1-ylmethyl)piperidin-1-ylcarbonyl]amino]propionate;
methyl (2S)-2-[1-(3,5-dichlorophenylsulfonyl)-L-prolylamino]-5-oxo-5-[4-[2-(4-phenylpiperazin-1-ylcarbonyloxy)ethyl]piperidin-1-yl]pentanoate;
methyl (2S)-2-[1-(3,5-dichlorophenylsulfonyl)-L-prolylamino]-5-oxo-5-[4-(2-propylimidazo[4,5-c]pyridin-1-ylmethyl)piperidin-1-yl]pentanoate;
(2S)-2-[1-(3,5-dichlorophenylsulfonyl)-L-prolyl]amino-3-[[4-[2-[(4-methylpiperazin-1-yl)carbonyloxy]ethyl]piperidin-1-ylcarbonyl]amino]propionic acid;
(2S)-2-[1-(3,5-dichlorophenylsulfonyl)-L-prolylamino]-5-oxo-5-[4-[2-(4-phenylpiperazin-1-ylcarbonyloxy)ethyl]piperidin-1-yl]pentanoic acid;
(2S)-2-[1-(3,5-dichlorophenylsulfonyl)-L-prolyl]amino-3-[[4-[2-[(4-methylpiperidin-1-yl)carbonyloxy]ethyl]piperidin-1-ylcarbonyl]amino]propionic acid;
(2S)-2-[1-(3,5-dichlorophenylsulfonyl)-L-prolylamino]-5-oxo-5-[4-(2-propylimidazo[4,5-c]pyridin-1-ylmethyl)piperidin-1-yl]pentanoic acid;
(2S)-2-[1-(3,5-dichlorophenylsulfonyl)-L-prolyl]amino-3-[[4-[2-[[4-(ethoxycarbonyl)piperazin-1-yl]carbonyloxy]ethyl]piperidin-1-ylcarbonyl]amino]propionic acid;
(2S)-2-[1-(3,5-dichlorophenylsulfonyl)-L-prolyl]amino-3-[[4-[2-[[4-(4-pyridyl)piperazin-1-yl]carbonyloxy]ethyl]piperidin-1-ylcarbonyl]amino]propionic acid;
(2S)-2-[1-(3,5-dichlorophenylsulfonyl)-L-prolyl]amino-3-[[4-[2-[(cis-2,6-dimethylmorpholin-4-yl)carbonyloxy]ethyl]piperidin-1-ylcarbonyl]amino]propionic acid;
(2S)-2-[1-(3,5-dichlorophenylsulfonyl)-L-prolyl]amino-3-[[4-(2-ethylimidazo[4,5-c]pyridin-1-ylmethyl)piperidin-1-ylcarbonyl]amino]propionic acid;
or a salt, solvate or prodrug thereof.

61. A process for preparing a compound of formula I according to claim 38, which comprises:
(a) when in a compound of formula I X represents —NR⁵—, reacting an amine of formula II with an acid of formula III 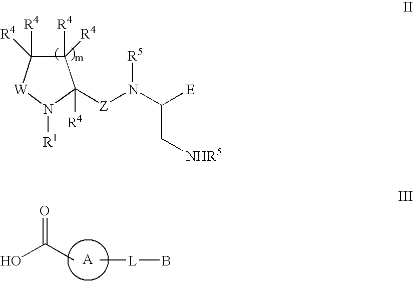
wherein R¹, R⁴, R⁵, W, Z, E, A, L, B and m have the meaning described in claim 38; or
(b) when in a compound of formula I X represents —O—, reacting an alcohol of formula IV with an acid of formula III 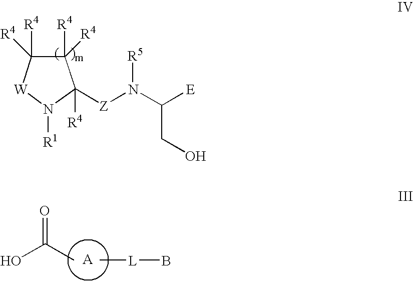
wherein R¹, R⁴, R⁵, W, Z, E, A, L, B and m have the meaning described in claim 38; or
(c) when in a compound of formula I X represents —CH₂— and A is bound to the carbonyl group through a nitrogen atom, reacting an acid of formula V with an amine of formula VI 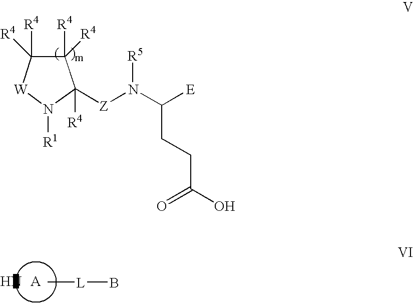
wherein R¹, R⁴, R⁵, W, Z, E, A, L, B and m have the meaning described in claim 38; or
(d) when in a compound of formula I X represents —NR⁵— and A is bound to the carbonyl group through a nitrogen atom, reacting an amine of formula II previously activated with an activating agent suitable for the preparation of ureas with an amine of formula VI 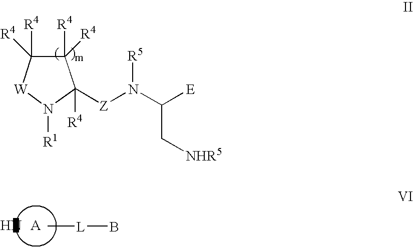
wherein R¹, R⁴, R⁵, W, Z, E, A, L, B and m have the meaning described in claim 38, or reacting an amine of formula VI previously activated with an activating agent suitable for the preparation of ureas with an amine of formula II, or alternatively reacting a compound of formula V′ previously activated with an azide suitable for a Curtius rearrangement with an amine of formula VI 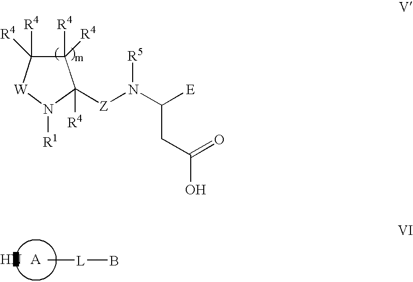
wherein R¹, R⁴, R⁵, W, Z, E, A, L, B and m have the meaning described in claim 38; or
(e) when in a compound of formula I X represents —O— and A is bound to the carbonyl group through a nitrogen atom, reacting an alcohol of formula IV previously activated with an activating agent suitable for the preparation of carbamates with an amine of formula VI 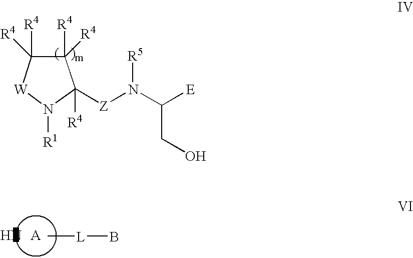
wherein R¹, R⁴, R⁵, W, Z, E, A, L, B and m have the meaning described in claim 38; or
(f) when in a compound of formula I Z represents —CO—, reacting an acid of formula VII with an amine of formula XVII 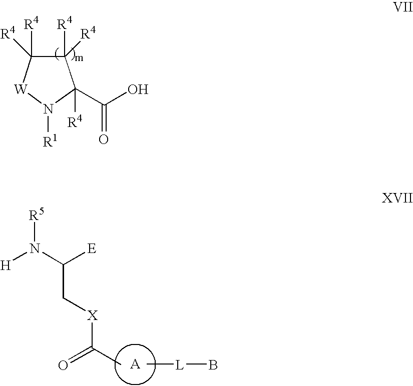
wherein R¹, R⁴, R⁵, W, E, X, A, L, B and m have the meaning described above; or
(g) when in a compound of formula I W represents —CR⁴R⁴— and R¹ represents —SO₂R², reacting a compound of formula XVIII with a sulfonyl chloride of formula IX 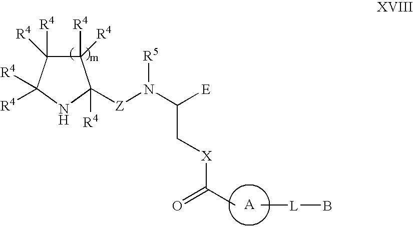
wherein R², R⁴, R⁵, Z, E, X, A, L, B and m have the meaning described in claim 38; or
(h) when in a compound of formula I W represents —CR⁴R⁴— and R¹ represents COR², reacting a compound of formula XVIII with an acid of formula X 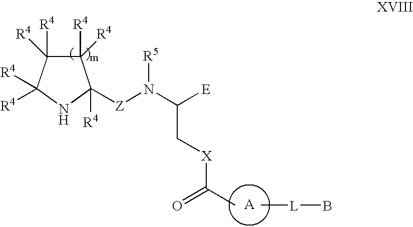
wherein R², R⁴, R⁵, Z, E, X, A, L, B and m have the meaning described in claim 38; or
(i) when in a compound of formula I W represents —CO— and R¹ represents —CH₂R³, reacting a compound of formula XIX with a compound of formula XI 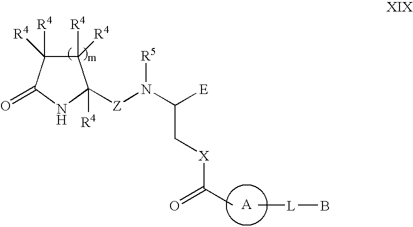
wherein R², R⁴, R⁵, Z, E, X, A, L, B and m have the meaning described in claim 38 and D represents a good leaving group; or
(j) when in a compound of formula I A is bound to the -L-B moiety through a ring nitrogen atom, alkylating the secondary amine of a compound of formula XX with a compound of formula XIV 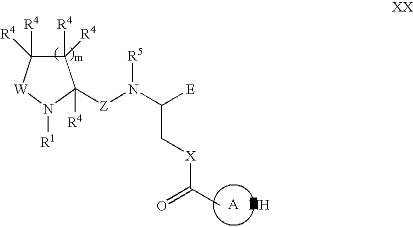
wherein R¹, R⁴, R⁵, W, Z, E, X, A, L, B and m have the meaning described in claim 38 and D represents a good leaving group; or
(k) transforming, in one or a plurality of steps, a compound of formula I into another compound of formula I; and
(l) if desired, after the above steps, reacting a compound of formula I with an acid or a base to give the corresponding addition salt.

62. A pharmaceutical composition which comprises an effective amount of a compound of formula I according to claim 38 or a pharmaceutically acceptable salt, solvate or prodrug thereof and one or more pharmaceutically acceptable excipients.

63. A method for the treatment or prevention of a disease mediated by integrins α₄ which comprises administering to a subject in need thereof an effective amount of a compound of formula I according to claim 38 or a pharmaceutically acceptable salt, solvate or prodrug thereof.

64. The method of claim 63, wherein the disease mediated by integrins α₄ is selected from inflammatory diseases, immune diseases, autoimmune diseases, degenerative disorders, tumor metastasis and ischemia-reperfusion disorders.

65. A method for the treatment or prevention of an inflammatory disease, immune disease or autoimmune disease which comprises administering to a subject in need thereof an effective amount of a compound of formula I according to claim 38 or a pharmaceutically acceptable salt, solvate or prodrug thereof.

66. The method of claim 65, wherein the inflammatory, immune or autoimmune disease is selected from diseases with an allergic component, inflammatory diseases with an autoimmune component, inflammatory bowel disease, inflammatory processes having an alloimmune origin caused by transplants or rejections, inflammatory processes that develop as a consequence of blood vessel revascularization treatments, encephalomyelitis, hepatitis, bronchitis, vasculitis and atherosclerosis.

67. The method of claim 66, wherein the disease with an allergic component is selected from asthma, allergic rhinitis, allergic dermatitis and allergic conjunctivitis.

68. The method of claim 66, wherein the inflammatory disease with an autoimmune component is selected from rheumatoid arthritis, psoriatic arthritis, multiple sclerosis, psoriasis and diabetes.

69. The method of claim 66, wherein the inflammatory bowel disease is selected from Crohn's disease and ulcerative colitis.

70. A method for the treatment or prevention of a degenerative disorder which comprises administering to a subject in need thereof an effective amount of a compound of formula I according to claim 38 or a pharmaceutically acceptable salt, solvate or prodrug thereof.

71. The method of claim 70, wherein the degenerative disorder is selected from Alzheimer's disease and arthrosis.

72. A method for the treatment or prevention of a tumor metastasis which comprises administering to a subject in need thereof an effective amount of a compound of formula I according to claim 38 or a pharmaceutically acceptable salt, solvate or prodrug thereof.

73. A method for the treatment or prevention of an ischemia-reperfusion disorder which comprises administering to a subject in need thereof an effective amount of a compound of formula I according to claim 38 or a pharmaceutically acceptable salt, solvate or prodrug thereof.

74. The method of claim 73, wherein the ischemia-reperfusion disorder is selected from acute coronary diseases and stroke.
